(19) United States
(12) Patent Application Publication
(10) Pub. No.: US 2024/0197680 A1
(43) Pub. Date: Jun. 20, 2024

(54) VORUCICLIB DOSING REGIMENS AND METHODS OF TREATMENT INCLUDING THE SAME

(71) Applicant: MEI Pharma, Inc., San Diego, CA (US)

(72) Inventors: Daniel GOLD, San Diego, CA (US); Alan F. PARR, Cary, NC (US)

(73) Assignee: MEI Pharma, Inc., San Diego, CA (US)

(21) Appl. No.: 18/554,497

(22) PCT Filed: Apr. 10, 2022
(86) PCT No.: PCT/US2022/024172

§ 371 Date: Oct. 9, 2023

Related U.S. Application Data
(60) Provisional application No. 63/173,361, filed on Apr. 10, 2021.
(60) Provisional application No. 63/173,398, filed on Apr. 10, 2021.

(51) Int. Cl. A61K 31/402; A61K 9/20; A61K 9/28; A61K 31/437; A61K 31/635; A61P 35/00; A61P 35/02

(52) U.S. Cl. CPC A61K 31/402; A61K 9/2009; A61K 9/2013; A61K 9/2018; A61K 9/2054; A61K 9/2059; A61K 9/28; A61K 31/437; A61K 31/635; A61P 35/00; A61P 35/02

(57) ABSTRACT

The present disclosure relates to a formulation comprising voruciclib malonate. The present disclosure further provides a method of treating a blood cancer using the formulation and an optional BCL-2 inhibitor, administered on various dosage regimens.

### CROSS-REFERENCE TO RELATED APPLICATIONS

The present application is an International Application which claims priority to U.S. Provisional Application Nos. 63/173,398, filed Apr. 10, 2021 and 63/173,361, filed Apr. 10, 2021, each of which is incorporated herein by reference in its entirety.

### FIELD

The disclosure provides novel methods of treatment using voruciclib and salts thereof.

### BACKGROUND

Numerous cancer-related therapeutics are under phase I or phase II clinical trial and evaluations at any particular time; however, most of them will fail to advance. In fact, it is estimated that more than 90% of cancer-related therapeutics will fail phase I or II clinical trial evaluation. The failure rate in phase III trials is almost 50%, and the cost of new drug development from discovery through phase III trials is between $0.8 billion and $1.7 billion and can take between eight and ten years. In addition, many patients fail to respond even to standard drugs that have been shown to be efficacious. For reasons that are not currently well understood or easily evaluated, individual patients may not respond to standard drug therapy. In some cases, administration of drug combinations may be more efficacious for treating cancer than drugs administered individually. These drug combinations may act synergistically to enhance the anti-cancer activity of the drugs. In some cases, drugs that are not particularly efficacious may find new and unexpected uses when combined with additional drug therapies.

### SUMMARY

The disclosure provides a formulation comprising between about 15% to about 35% w/w voruciclib malonate and one or more pharmaceutically acceptable excipients. In some embodiments, the formulation comprises between about 18% to about 30% w/w voruciclib malonate. In some embodiments, the formulation comprises about 18%, about 19%, about 20%, about 21%, about 22%, about 23%, about 24%, about 25%, about 26%, about 27%, or about 28% w/w voruciclib malonate. In some embodiments, the formulation comprises between about 20% to about 23% w/w voruciclib malonate. In some embodiments, the one or more pharmaceutically acceptable excipients comprise about 5% to about 37% w/w microcrystalline cellulose. In some embodiments, the one or more pharmaceutically acceptable excipients comprise about 10%, about 11%, about 12%, about 13%, about 14%, about 15%, about 16%, about 17%, about 18%, about 19%, or about 20% w/w microcrystalline cellulose. In some embodiments, the one or more pharmaceutically acceptable excipients comprise about 1% to about 48% w/w lactose monohydrate. In some embodiments, the one or more pharmaceutically acceptable excipients comprise about 2%, about 3%, about 4%, about 5%, about 6%, about 7%, about 8%, about 9%, about 10%, about 11%, about 12%, about 13%, about 14%, or about 15% w/w lactose monohydrate. In some embodiments, the one or more pharmaceutically acceptable excipients comprise about 20% to about 70% w/w dibasic calcium phosphate dihydrate. In some embodiments, the one or more pharmaceutically acceptable excipients comprise about 34%, about 35%, about 36%, about 37%, about 38%, about 39%, about 40%, about 41%, about 42%, about 43%, about 44%, about 45%, about 46%, about 47%, about 48%, about 49%, or about 50% w/w dibasic calcium phosphate dihydrate. In some embodiments, the one or more pharmaceutically acceptable excipients comprise about 0.1% to about 15% w/w sodium bicarbonate. In some embodiments, the one or more pharmaceutically acceptable excipients comprise about 2%, about 3%, about 4%, about 5%, about 6%, about 7%, about 8%, about 9%, or about 10% w/w sodium bicarbonate. In some embodiments, the one or more pharmaceutically acceptable excipients comprise about 1% to about 20% w/w sodium starch glycolate. In some embodiments, the one or more pharmaceutically acceptable excipients comprise about 2%, about 3%, about 4%, about 5%, about 6%, about 7%, about 8%, about 9%, about 10%, about 11%, or about 12% w/w sodium starch glycolate. In some embodiments, the one or more pharmaceutically acceptable excipients comprise about 0.01% to about 10% w/w magnesium stearate. In some embodiments, the one or more pharmaceutically acceptable excipients comprise about 0.1%, about 0.25%, about 0.5%, about 0.75%, about 1%, about 1.25%, about 1.5%, about 1.75%, about 2%, about 3%, about 4%, or about 5% w/w magnesium stearate. In some embodiments, the one or more pharmaceutically acceptable excipients comprise about 0.01% to about 10% w/w colloidal silicon dioxide. In some embodiments, the one or more pharmaceutically acceptable excipients comprise about 0.1%, about 0.25%, about 0.5%, about 0.75%, about 1%, about 1.25%, about 1.5%, about 1.75%, about 2%, about 3%, about 4%, or about 5% w/w colloidal silicon dioxide. In some embodiments, the formulation is comprised into a tablet. In some embodiments, the tablet is coated with a film coating. In some embodiments, voruciclib malonate comprises a crystal form of voruciclib malonate characterized by an X-ray powder diffraction pattern comprising one or more peaks selected from 7.30°±0.2°, 13.58°±0.2°, 14.06°±0.2°, 15.18°±0.2°, 15.66°±0.2°, 17.50°±0.2°, 18.94°±0.2°, 19.54°±0.2°, 22.22°±0.2°, 23.38°±0.2°, 24.10°±0.2°, 24.98°±0.2°, 25.94°±0.2°, 27.26°±0.2°, 28.50°±0.2°, and 32.82°±0.2° 2θ. In some embodiments, voruciclib malonate comprises a crystal form of voruciclib malonate characterized by an X-ray powder diffraction pattern comprising one or more peaks selected from 6.36°±0.2° 2θ, 13.88°±0.2° 2θ, 7.31°±0.2° 2θ, 9.34°±0.2° 2θ, 10.05°±0.2° 2θ, 13.59°±0.2° 2θ, 14.08°±0.2° 2θ, 15.21°±0.2° 2θ, 15.67°±0.2° 2θ, 17.53°±0.2° 2θ, 18.70°±0.2° 2θ, 18.98°±0.2° 2θ, 19.38°±0.2° 2θ, 19.67°±0.2° 2θ, 20.16°±0.2° 2θ, 20.39°±0.2° 2θ, 21.01°±0.2° 20, 22.27°±0.2° 2θ, 23.35°±0.2° 2θ, 24.15°±0.2° 2θ, 24.67°±0.2° 2θ, 25.00°±0.2° 2θ, 25.18°±0.2° 2θ, 25.57°±0.2° 2θ, 25.93º=0.2° 2θ, 26.21°±0.2° 2θ, 27.19°±0.2° 2θ, and 27.38°±0.2° 2θ. In some embodiments, the crystal form is a crystalline anhydrate. In some embodiments, the crystal form is a crystalline hydrate.

The disclosure provides a method of treating a disease or disorder in a subject, the method comprising administering to the subject a therapeutically effective of a formulation described herein. The disclosure also provides a method of treating a disease or disorder in a subject, the method comprising administering to the subject a therapeutically effective of a formulation comprising between about 15% to 35% w/w voruciclib malonate, about 5% to 37% w/w microcrystalline cellulose, about 1% to about 48% w/w lactose monohydrate, about 20% to about 70% w/w dibasic calcium phosphate dihydrate, about 0.1% to about 15% w/w sodium bicarbonate, about 1% to about 20% w/w sodium starch glycolate, and about 0.01% to about 10% w/w magnesium stearate. In some embodiments, the voruciclib malonate comprises a crystal form of voruciclib malonate characterized by an X-ray powder diffraction pattern comprising one or more peaks selected from 7.30°±0.2°, 13.58°±0.2°, 14.06°±0.2°, 15.18°±0.2°, 15.66°±0.2°, 17.50°±0.2°, 18.94°±0.2°, 19.54°±0.2°, 22.22°±0.2°, 23.38°±0.2°, 24.10°±0.2°, 24.98°±0.2°, 25.94°±0.2°, 27.26°±0.2°, 28.50°±0.2°, and 32.82°±0.2° 2θ. In some embodiments, the voruciclib malonate comprises a crystal form of voruciclib malonate characterized by an X-ray powder diffraction pattern comprising one or more peaks selected from 6.36°±0.2° 2θ, 13.88°±0.2° 2θ, 7.31°±0.2° 2θ, 9.34°±0.2° 2θ, 10.05°±0.2° 2θ, 13.59°±0.2° 2θ, 14.08°±0.2° 2θ, 15.21°±0.2° 2θ, 15.67°±0.2° 2θ, 17.53°±0.2° 2θ, 18.70°±0.2° 2θ, 18.98°±0.2° 2θ, 19.38°±0.2° 2θ, 19.67°±0.2° 2θ, 20.16°±0.2° 2θ, 20.39°±0.2° 2θ, 21.01°±0.2° 2θ, 22.27°±0.2° 2θ, 23.35°±0.2° 2θ, 24.15°±0.2° 2θ, 24.67°±0.2° 2θ, 25.00°±0.2° 2θ, 25.18°±0.2° 2θ, 25.57°±0.2° 2θ, 25.93°±0.2° 2θ, 26.21°±0.2° 2θ, 27.19°±0.2° 2θ, and 27.38°±0.2° 2θ. In some embodiments, the formulation comprises about 0.01% to about 10% w/w colloidal silicon dioxide. In some embodiments, the formulation is comprised into a tablet and the tablet is coated with a film coating. In some embodiments, the disease or disorder is a blood cancer. In some embodiments, the blood cancer is selected from acute myeloid leukemia (AML), chronic myeloid leukemia (CML), acute lymphocytic lymphoma (ALL), and chronic lymphocytic leukemia (CLL). In some embodiments, the formulation is administered to the subject such that subject receives a daily voruciclib dose between about 50 mg and about 100 mg, between about 100 mg and about 150 mg, between about 150 mg and about 200 mg, between about 200 mg and about 250 mg, between about 250 mg and about 300 mg, between about 300 mg and about 350 mg, between about 350 mg and about 400 mg, between about 400 mg and about 450 mg, between about 450 mg and about 500 mg, between about 500 mg and about 550 mg, between about 550 mg and about 600 mg, between about 600 mg and about 650 mg, between about 650 mg and about 700 mg, between about 700 mg and about 750 mg, between about 750 mg and about 800 mg, between about 800 mg and about 850 mg, between about 850 mg and about 900 mg, between about 900 mg and about 950 mg, or between about 950 mg and about 1,000 mg. In some embodiments, the formulation is administered to the subject such that subject receives a daily voruciclib dose of about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1,000 mg. In some embodiments, the formulation is administered to the subject such that subject receives a daily voruciclib dose of about 200 mg or about 250 mg. In some embodiments, the formulation is administered to the subject such that subject receives a daily voruciclib dose not exceeding 350 mg. In some embodiments, the voruciclib dose is a voruciclib free base dose. In some embodiments, the formulation is administered to the subject daily for about one day, about two days, about three days, about 4 days, about 5 days, about 6 days, about 7 days, about 8 days, about 9 days, about 10 days, about 11 days, about 12 days, about 13 days, or about 14 days. In some embodiments, the formulation is administered to the subject daily for about one week, about two weeks, about three weeks, or about 4 weeks. In some embodiments, administration of the formulation is paused for about one day, about two days, about three days, about 4 days, about 5 days, about 6 days, about 7 days, about 8 days, about 9 days, about 10 days, about 11 days, about 12 days, about 13 days, or about 14 days. In some embodiments, administration of the formulation is paused for about one week, about two weeks, about three weeks, or about 4 weeks. In some embodiments, the formulation is administered to the subject on a 14 days on/14 days off schedule. In some embodiments, the formulation is administered for about one month, about two months, about three months, about 4 months, about 5 months, about 6 months, about 7 months, about 8 months, about 9 months, about 10 months, about 11 months, or about 12 months. In some embodiments, the formulation is administered in combination with a BCL-2 inhibitor. In some embodiments, the BCL-2 inhibitor is selected from navitoclax, venetoclax, A-1155463, A-1331852, ABT-737, obatoclax, S44563, TW-37, A-1210477, AT101, HA14-1, BAM7, sabutoclax, UMI-77, gambogic acid, maritoclax, MIMI, methylprednisolone, iMAC2, Bax inhibitor peptide V5, Bax inhibitor peptide P5, Bax channel blocker, ARRY 520 trifluoroacetate, or a pharmaceutically acceptable salt of any one thereof. In some embodiments, the BCL-2 inhibitor is venetoclax or a pharmaceutically acceptable salt thereof. In some embodiments, the BCL-2 inhibitor is administered to the subject daily for about one day, about two days, about three days, about 4 days, about 5 days, about 6 days, about 7 days, about 8 days, about 9 days, about 10 days, about 11 days, about 12 days, about 13 days, or about 14 days. In some embodiments, the BCL-2 inhibitor is administered to the subject daily for about one week, about two weeks, about three weeks, or about 4 weeks. In some embodiments, administration of the BCL-2 inhibitor is paused for about one day, about two days, about three days, about 4 days, about 5 days, about 6 days, about 7 days, about 8 days, about 9 days, about 10 days, about 11 days, about 12 days, about 13 days, or about 14 days. In some embodiments, administration of the BCL-2 inhibitor is paused for about one week, about two weeks, about three weeks, or about 4 weeks. In some embodiments, the BCL-2 inhibitor is administered to the subject on a 14 days on/14 days off schedule. In some embodiments, the BCL-2 inhibitor is administered to the subject for about one month, about two months, about three months, about 4 months, about 5 months, about 6 months, about 7 months, about 8 months, about 9 months, about 10 months, about 11 months, or about 12 months.

### BRIEF DESCRIPTION OF THE DRAWINGS

The foregoing summary, as well as the following detailed description of the disclosure, will be better understood when read in conjunction with the appended drawings.

FIGS. 1A and 1B illustrate decreased c-MYC Expression in Solid Tumors: 10 gene biomarkers evaluated in Phase 1 daily dosing study; c-MYC expression decreased in 17/25 patients (68%).

FIGS. 2A-2C illustrate CR in a Patient with Pulmonary Metastases; FIG. 2A: baseline CT scan; FIG. 2B: 2 months after starting the trial, radiological CR based on official radiological report; FIG. 2C: 14 months after starting the trail, patient remained on trial for 12 months only, and CR remained durable for 14 months.

FIGS. 3A-3D illustrate that Voruciclib Shows Preferential Tumor Accumulation in a Preclinical Model.

FIGS. 4A and 4B illustrate that Voruciclib Synergizes with Venetoclax in Venetoclax Sensitive and Resistant Cell Lines.

FIG. 5 is a manufacturing process flow diagram of voruciclib malonate tablets

FIG. 6 is a chromatogram for dissolution testing of voruciclib malonate tablets, 50 mg.

FIG. 7 is a graph depicting the dissolution of voruciclib hydrochloride capsules and voruciclib malonate tablets.

FIG. 8A is the molecular structure of voruciclib malonate (with stereochemistry).

FIG. 8B is the X-ray crystal structure of voruciclib malonate drug substance (Batch 20-07211).

FIG. 9 is an overlay of HR-XRPD patterns (bulk and simulated for single crystal) wherein the simulated XRPD pattern was generated using mercury software from Cambridge Crystallographic Data Center based on the atom positions obtained from the single crystal analysis.

FIG. 10 is the XRPD diffractogram of voruciclib malonate.

FIG. 11 is a synthetic scheme for the synthesis of voruciclib malonate.

FIG. 12 is a flow diagram for the manufacturing process of voruciclib malonate. Abbreviations: RSM=Starting material; Ac₂O=acetic anhydride; DCM=dichloromethane; DM=dimethylformamide; Et₃N=triethylamine; NaH=sodium hydride; 2-methyl-THF=2-methyltetrahydrofuran; BF₃Et₂O=boron trifluoride diethyl etherate; IPA=Isopropyl alcohol

FIGS. 13A-13F depict the chemical transformations for the manufacture of voruciclib malonate. FIG. 13A depicts the synthesis of Intermediate 1. FIG. 13B depicts the synthesis of Intermediate 2. FIG. 13C depicts the synthesis of Intermediate 3. FIG. 13D depicts the synthesis of Intermediate 4. FIG. 13E depicts the synthesis of Intermediate 5. FIG. 13F depicts the synthesis of voruciclib malonate from Intermediate 5.

### DETAILED DESCRIPTION

While preferred embodiments of the disclosure are shown and described herein, such embodiments are provided by way of example only and are not intended to otherwise limit the scope of the disclosure. Various alternatives to the described embodiments of the disclosure may be employed in practicing the disclosure.

### Definitions

Unless defined otherwise, all technical and scientific terms used herein have the same meaning as is commonly understood by one of skill in the art to which this disclosure belongs. All patents and publications referred to herein are incorporated by reference in their entireties.

The term “solid form” may refer to a crystalline solid form or phase, including a crystalline free base and a crystalline salt.

The terms “co-administration,” “co-administering,” “administered in combination with,” and “administering in combination with” as used herein, encompass administration of two or more agents to a subject so that both agents and/or their metabolites are present in the subject at the same time. Co-administration includes simultaneous administration in separate compositions, administration at different times in separate compositions, or administration in a composition in which two or more agents are present.

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, the manner of administration, etc. which can readily be determined by one of ordinary skill in the art. The term also applies to a dose that will induce a particular response in target cells (e.g., CDK inhibition). The specific dose will vary depending on the particular compounds chosen, the dosing regimen to be followed, whether the compound is administered in combination with other compounds, timing of administration, the tissue to which it is administered, and the physical delivery system in which the compound is carried.

The term “synergistic,” or “synergistic effect” or “synergism” as used herein, generally refers to an effect such that the one or more effects of the combination of compositions is greater than the one or more effects of each component alone, or they can be greater than the sum of the one or more effects of each component alone. The synergistic effect can be greater than about 10%, 20%, 30%, 50%, 75%, 100%, 110%, 120%, 150%, 200%, 250%, 350%, or 500% or more than the effect on a subject with one of the components alone, or the additive effects of each of the components when administered individually. The effect can be any of the measurable effects described herein. Advantageously, such synergy between the agents when combined, may allow for the use of smaller doses of one or both agents, may provide greater efficacy at the same doses, and may prevent or delay the build-up of multi-drug resistance. The combination index (CI) method of Chou and Talalay may be used to determine the synergy, additive or antagonism effect of the agents used in combination. When the CI value is less than 1, there is synergy between the compounds used in the combination; when the CI value is equal to 1, there is an additive effect between the compounds used in the combination and when CI value is more than 1, there is an antagonistic effect. The synergistic effect may be attained by co-formulating the agents of the pharmaceutical combination. The synergistic effect may be attained by administering two or more agents as separate formulations administered simultaneously or sequentially.

Cyclin-dependent kinases (CDKs) are a family of enzymes which become activated in specific phases of the cell cycle. CDKs consist of a catalytic subunit (the actual cyclin-dependent kinase or CDK) and a regulatory subunit (cyclin). There are at least nine CDKs (CDK1, CDK2, CDK3, CDK4, CDK5, CDK6, CDK7, CDK8, CDK9, etc.) and at least 15 different types of cyclins (cyclin A, B1, B2, D1, D2, D3, E, H etc.). Each step of the cell cycle is regulated by such CDK complexes: G1/S transition (CDK2/cyclin A, CDK4/cyclin DI-D3, CDK6/cyclin D3), S phase (CDK2/cyclin A), G2 phase 30 (CDK1/cyclin A), G2 M transition phase (CDK1/cyclin B).

As used herein, the term “CDK inhibitor” refers to an agent that is capable of inhibiting one or more cyclin dependent kinases (CDK). Aberrant expression and overexpression of these kinases are evidenced in many disease conditions such as cancer. In the context of the present invention, the CDK inhibitor of the pharmaceutical combination described herein may be a compound of Formula I. Ia, or Ib or a pharmaceutically acceptable salt thereof. The compounds of the present disclosure may inhibit one or more of CDK1/cyclin B, CDK2/cyclin E, CDK4/cyclin D, CDK4/cyclin DI and CDK9/cyclin TI with specificity. In certain embodiments, a compound of the disclosure inhibits CDK9/cyclin TI or CDK9 with specificity.

Disclosed herein are combination therapies for the treatment of cancer, e.g., leukemia, lymphoma and breast cancer. The methods and compositions described herein may include a cyclin-dependent kinase (CDK) inhibitor, such as a compound of Formula I. Ia, or Ib or a pharmaceutically acceptable salt thereof. In some cases, a combination therapy may include a CDK inhibitor in combination with a proteasome inhibitor. In other cases, a combination therapy may include a CDK inhibitor in combination with a BCL-2 inhibitor.

In certain embodiments, a CDK inhibitor of the disclosure is represented by a compound disclosed in U.S. Pat. Nos. 7,271,193; 7,915,301; 8,304,449; 7,884,127; 8,563,596, the entire contents of each of which are incorporated herein by reference.

The terms “QD,” “qd,” or “q.d.” mean quaque die, once a day, or once daily. The terms “BID,” “bid,” or “b.i.d.” mean bis in die, twice a day, or twice daily. The terms “TID,” “tid,” or “t.i.d.” mean ter in die, three times a day, or three times daily. The terms “QID,” “qid,” or “q.i.d.” mean quater in die, four times a day, or four times daily.

A “therapeutic effect” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

As used herein, the term “free base dosage” or “free base dose” refers to an amount of drug in its free base form, which can be replaced by an amount of a salt of the drug by using a salt conversion factor.

The term “pharmaceutically acceptable salt” refers to salts derived from a variety of organic and inorganic counter ions, including fumarate, maleate, phosphate, L-tartrate, esylate, besylate, hydrobromide, hydrochloride, citrate, gentisate, oxalate, sulfate counter ions, and the like. Pharmaceutically acceptable acid addition salts can be formed with inorganic acids and organic acids.

“Pharmaceutically acceptable carrier” or “pharmaceutically acceptable excipient” is intended to include any and all solvents, dispersion media, coatings, antibacterial and antifungal agents, isotonic and absorption delaying agents. Except insofar as any conventional media or agent is incompatible with the active ingredient, its use in the therapeutic compositions of the disclosure is contemplated. Supplementary active ingredients can also be incorporated into the described compositions.

The term “in vivo” refers to an event that takes place in a subject's body.

The term “in vitro” refers to an event that takes places outside of a subject's body. In vitro assays encompass cell-based assays in which cells alive or dead are employed and may also encompass a cell-free assay in which no intact cells are employed.

The term “extragranular” refers to substances that are outside of a granule, e.g., a substance added to granules (multiparticle compacts formed by a granulation process) and physically mixed with granules, but not contained within the granules.

The term “intragranular” refers to substances that are within a granule (a multiparticle compact formed by a granulation process). Granules may be formed by processes such as wet granulation (i.e., prepared using moisture or steam, thermal, melt, freeze, foam, and other processes) or dry granulation.

The term “acidulant” refers to a substance that increases acidity.

The terms “transmission” or “transmission mode,” when used in conjunction with powder X-ray diffraction, refers to the transmission (also known as Debye-Scherrer) sampling mode. The terms “reflection” or “reflection mode,” when used in conjunction with powder X-ray diffraction, refers to the reflection (also known as Bragg-Brentano) sampling mode.

Unless otherwise stated, the chemical structures depicted herein are intended to include compounds which differ only in the presence of one or more isotopically enriched atoms. For example, compounds where one or more hydrogen atoms is replaced by deuterium or tritium, or wherein one or more carbon atoms is replaced by ¹³C- or ¹⁴C-enriched carbons, are within the scope of this disclosure.

When ranges are used herein to describe, for example, physical or chemical properties such as molecular weight or chemical formulae, all combinations and subcombinations of ranges and specific embodiments therein are intended to be included. Use of the term “about” or “approximately” when referring to a number or a numerical range means that the number or numerical range referred to is an approximation within experimental variability (or within statistical experimental error), and thus the number or numerical range may vary from, for example, between 1% and 15% of the stated number or numerical range. The term “comprising” (and related terms such as “comprise” or “comprises” or “having” or “including”) includes those embodiments such as, for example, an embodiment of any composition of matter, method or process that “consist of” or “consist essentially of” the described features.

“Enantiomeric purity” as used herein refers to the relative amounts, expressed as a percentage, of the presence of a specific enantiomer relative to the other enantiomer. For example, if a compound, which may potentially have an (R)- or an (S)-isomeric configuration, is present as a racemic mixture, the enantiomeric purity is about 50% with respect to either the (R)- or (S)-isomer. If that compound has one isomeric form predominant over the other, for example, 80% (S)-isomer and 20% (R)-isomer, the enantiomeric purity of the compound with respect to the (S)-isomeric form is 80%. The enantiomeric purity of a compound can be determined in a number of ways, including but not limited to chromatography using a chiral support, polarimetric measurement of the rotation of polarized light, nuclear magnetic resonance spectroscopy using chiral shift reagents which include but are not limited to lanthanide containing chiral complexes or Pirkle's reagents, or derivatization of a compounds using a chiral compound such as Mosher's acid followed by chromatography or nuclear magnetic resonance spectroscopy.

In preferred embodiments, the enantiomerically enriched composition has a higher potency with respect to therapeutic utility per unit mass than does the racemic mixture of that composition. Enantiomers can be isolated from mixtures by methods known to those skilled in the art, including chiral high pressure liquid chromatography (HPLC) and the formation and crystallization of chiral salts; or preferred enantiomers can be prepared by asymmetric syntheses. See, for example, Jacques, et al., Enantiomers. Racemates and Resolutions, Wiley Interscience, New York, 1981; Eliel, Stereochemistry of Carbon Compounds, McGraw-Hill, N Y. 1962; and Eliel and Wilen, Stereochemistry of Organic Compounds, Wiley-Interscience, New York, 1994.

The terms “enantiomerically enriched” and “non-racemic,” as used herein, refer to compositions in which the percent by weight of one enantiomer is greater than the amount of that one enantiomer in a control mixture of the racemic composition (e.g., greater than 1:1 by weight). For example, an enantiomerically enriched preparation of the (S)-enantiomer, means a preparation of the compound having greater than 50% by weight of the (S)-enantiomer relative to the (R)-enantiomer, such as at least 75% by weight, or such as at least 80% by weight. In some embodiments, the enrichment can be significantly greater than 80% by weight, providing a “substantially enantiomerically enriched” or a “substantially non-racemic” preparation, which refers to preparations of compositions which have at least 85% by weight of one enantiomer relative to other enantiomer, such as at least 90% by weight, or such as at least 95% by weight. The terms “enantiomerically pure” or “substantially enantiomerically pure” refers to a composition that comprises at least 98% of a single enantiomer and less than 2% of the opposite enantiomer.

“Moiety” refers to a specific segment or functional group of a molecule. Chemical moieties are often recognized chemical entities embedded in or appended to a molecule.

“Tautomers” are structurally distinct isomers that interconvert by tautomerization. “Tautomerization” is a form of isomerization and includes prototropic or proton-shift tautomerization, which is considered a subset of acid-base chemistry. “Prototropic tautomerization” or “proton-shift tautomerization” involves the migration of a proton accompanied by changes in bond order, often the interchange of a single bond with an adjacent double bond. Where tautomerization is possible (e.g., in solution), a chemical equilibrium of tautomers can be reached. An example of tautomerization is keto-enol tautomerization. A specific example of keto-enol tautomerization is the interconversion of pentane-2,4-dione and 4-hydroxypent-3-en-2-one tautomers. Another example of tautomerization is phenol-keto tautomerization. The formation of solid forms in different tautomerization states is known as “desmotropy” and such forms are known as “desmotropes.”

Compositions of the disclosure also include crystalline forms of Formula (1), including, for example, polymorphs, pseudopolymorphs, solvates, hydrates, unsolvated polymorphs (including anhydrates), and conformational polymorphs, as well as mixtures thereof. “Crystalline form”, “form,” and “polymorph” are intended to include all crystalline forms of the compound, including, for example, polymorphs, pseudopolymorphs, solvates, hydrates, unsolvated polymorphs (including anhydrates), and conformational polymorphs, as well as mixtures thereof, unless a particular crystalline form is referred to.

“Solvate” refers to a crystalline phase of a compound in physical association with one or more molecules of a solvent. The crystalline phase of a compound in physical association with one or more molecules of water is referred to as a “hydrate.”

“Amorphous form” refers to a form of a compound, or a salt or molecular complex of a compound, that lacks long range crystalline order.

### Voruciclib

Voruciclib is a CDK inhibitor described for example in U.S. Pat. Nos. 7,271,193, 7,915,301, 8,304,449, 7,884,127, and 8,563,596, incorporated herein by reference in their entireties.

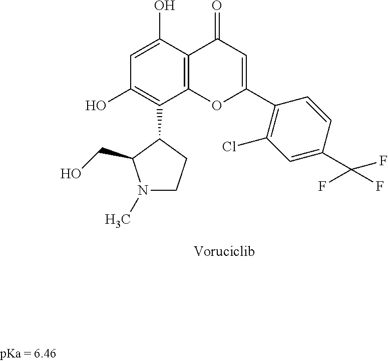 [SMILES: CN1CC[C@@H](c2c(O)cc(O)c3c(=O)cc(-c4ccc(C(F)(F)F)cc4Cl)oc23)[C@@H]1CO]

In some embodiments, voruciclib refers to (+)-trans-2-(2-chloro-4-trifluoromethylphenyl)-5,7-dihydroxy-8-(2-hydroxymethyl-1-methylpyrrolidin-3-yl)-chromen-4-one. In some embodiments, voruciclib refers to 2-(2-chloro-4-trifluoromethylphenyl)-5,7-dihydroxy-8-((2R,3S)-2-hydroxymethyl-1-methylpyrrolidin-3-yl)-4H-chromen-4-one.

Voruciclib salts and/or polymorphs thereof, and methods of making and using thereof are described for example in International Patent Application Publication WO 2020/210760, incorporated herein in its entirety.

The present disclosure provides pharmaceutically-acceptable salts of any compound described herein, e.g., voruciclib, BCL-2 inhibitors, proteasome inhibitors, and the like. Pharmaceutically-acceptable salts include, for example, acid-addition salts and base-addition salts. The acid that is added to a compound to form an acid-addition salt can be an organic acid or an inorganic acid. A base that is added to a compound to form a base-addition salt can be an organic base or an inorganic base. In some cases, a pharmaceutically-acceptable salt is a metal salt. In some cases, a pharmaceutically-acceptable salt is an ammonium salt.

Acid addition salts can arise from the addition of an acid to a compound described herein. In some cases, the acid is organic. In some cases, the acid is inorganic. Non-limiting examples of suitable acids include hydrochloric acid, hydrobromic acid, hydroiodic acid, nitric acid, nitrous acid, sulfuric acid, sulfurous acid, a phosphoric acid, nicotinic acid, isonicotinic acid, lactic acid, salicylic acid, 4-aminosalicylic acid, tartaric acid, ascorbic acid, gentisinic acid, gluconic acid, glucaronic acid, saccaric acid, formic acid, benzoic acid, glutamic acid, pantothenic acid, acetic acid, propionic acid, butyric acid, fumaric acid, succinic acid, citric acid, oxalic acid, malonic acid, maleic acid, hydroxymaleic acid, methylmaleic acid, glycolic acid, malic acid, cinnamic acid, mandelic acid, 2-phenoxy benzoic acid, 2-acetoxy benzoic acid, embonic acid, phenylacetic acid, N-cyclohexylsulfamic acid, methanesulfonic acid, ethanesulfonic acid, benzenesulfonic acid, p-toluenesulfonic acid, 2-hydroxyethanesulfonic acid, ethane-1,2-disulfonic acid, 4-methylbenzenesulfonic acid, naphthalene-2-sulfonic acid, naphthalene-1,5-disulfonic acid, 2-phosphoglyceric acid, 3-phosphoglyceric acid, glucose-6-phosphoric acid, and an amino acid.

Metal salts can arise from the addition of an inorganic base to a compound of the invention. The inorganic base consists of a metal cation paired with a basic counterion, such as, for example, hydroxide, carbonate, bicarbonate, or phosphate. The metal can be an alkali metal, alkaline earth metal, transition metal, or main group metal. In some embodiments, the metal is lithium, sodium, potassium, cesium, cerium, magnesium, manganese, iron, calcium, strontium, cobalt, titanium, aluminum, copper, cadmium, or zinc.

In some embodiments, a metal salt is a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, or a zinc salt.

Ammonium salts can arise from the addition of ammonia or an organic amine to a compound described herein. Non-limiting examples of suitable organic amines include triethyl amine, diisopropyl amine, ethanol amine, diethanol amine, triethanol amine, morpholine, N-methylmorpholine, piperidine, N-methylpiperidine, N-ethylpiperidine, dibenzyl amine, piperazine, pyridine, pyrrazole, pipyrrazole, imidazole, pyrazine, pipyrazine, ethylenediamine, N,N′-dibenzylethylene diamine, procaine, chloroprocaine, choline, dicyclohexyl amine, and N-methylglucamine.

Non-limiting examples of suitable ammonium salts include is a triethyl amine salt, a diisopropyl amine salt, an ethanol amine salt, a diethanol amine salt, a triethanol amine salt, a morpholine salt, an N-methylmorpholine salt, a piperidine salt, an N-methylpiperidine salt, an N-ethylpiperidine salt, a dibenzyl amine salt, a piperazine salt, a pyridine salt, a pyrrazole salt, a pipyrrazole salt, an imidazole salt, a pyrazine salt, a pipyrazine salt, an ethylene diamine salt, an N,N′-dibenzylethylene diamine salt, a procaine salt, a chloroprocaine salt, a choline salt, a dicyclohexyl amine salt, and a N-methylglucamine salt.

Non-limiting examples of suitable acid addition salts include a hydrochloride salt, a hydrobromide salt, a hydroiodide salt, a nitrate salt, a nitrite salt, a sulfate salt, a sulfite salt, a phosphate salt, a hydrogen phosphate salt, a dihydrogen phosphate salt, a carbonate salt, a bicarbonate salt, a nicotinate salt, an isonicotinate salt, a lactate salt, a salicylate salt, a 4-aminosalicylate salt, a tartrate salt, an ascorbate salt, a gentisinate salt, a gluconate salt, a glucaronate salt, a saccarate salt, a formate salt, a benzoate salt, a glutamate salt, a pantothenate salt, an acetate salt, a propionate salt, a butyrate salt, a fumarate salt, a succinate salt, a citrate salt, an oxalate salt, a maleate salt, a hydroxymaleate salt, a methylmaleate salt, a glycolate salt, a malate salt, a cinnamate salt, a mandelate salt, a 2-phenoxy benzoate salt, a 2-acetoxy benzoate salt, an embonate salt, a phenylacetate salt, an N-cyclohexylsulfamate salt, a methanesulfonate salt, an ethanesulfonate salt, a benzenesulfonate salt, a p-toluenesulfonate salt, a 2-hydroxyethanesulfonate salt, an ethane-1,2-disulfonate salt, a 4-methylbenzenesulfonate salt, a naphthalene-2-sulfonate salt, a naphthalene-1,5-disulfonate salt, a 2-phosphoglycerate salt, a 3-phosphoglycerate salt, a glucose-6-phosphate salt, and an amino acid salt.

In some aspects, combinations described herein, e.g., combinations of CDK inhibitors with BCL-2 inhibitors or proteasome inhibitors, can be utilized for the treatment of cancer. A combination therapy described herein can reduce the likelihood of metastasis in a subject in need thereof. In some embodiments, the metastasis is a solid tumor. In some embodiments, the metastasis is a liquid tumor. Cancers that are liquid tumors can be those that occur, for example, in blood, bone marrow, and lymph nodes, and can include, for example, leukemia, myeloid leukemia, lymphocytic leukemia, lymphoma. Hodgkin's lymphoma, melanoma, and multiple myeloma. Leukemias include, for example, acute lymphoblastic leukemia (ALL), acute myeloid leukemia (AML), chronic lymphocytic leukemia (CLL), chronic myelogenous leukemia (CML), and hairy cell leukemia. Cancers that are solid tumors include, for example, prostate cancer, testicular cancer, breast cancer, brain cancer, pancreatic cancer, colon cancer, thyroid cancer, stomach cancer, lung cancer, ovarian cancer. Kaposi's sarcoma, skin cancer, squamous cell skin cancer, renal cancer, head and neck cancers, throat cancer, squamous carcinomas that form on the moist mucosal linings of the nose, mouth, throat, bladder cancer, osteosarcoma, cervical cancer, endometrial cancer, esophageal cancer, liver cancer, and kidney cancer. In some embodiments, the condition treated by the methods described herein is metastasis of melanoma cells, prostate cancer cells, testicular cancer cells, breast cancer cells, brain cancer cells, pancreatic cancer cells, colon cancer cells, thyroid cancer cells, stomach cancer cells, lung cancer cells, ovarian cancer cells. Kaposi's sarcoma cells, skin cancer cells, renal cancer cells, head or neck cancer cells, throat cancer cells, squamous carcinoma cells, bladder cancer cells, osteosarcoma cells, cervical cancer cells, endometrial cancer cells, esophageal cancer cells, liver cancer cells, or kidney cancer cells.

The methods described herein can also be used for inhibiting progression of metastatic cancer tumors. Non-limiting examples of cancers include adrenocortical carcinoma, childhood adrenocortical carcinoma. AIDS-related cancers, anal cancer, appendix cancer, basal cell carcinoma, childhood basal cell carcinoma, bladder cancer, childhood bladder cancer, bone cancer, brain tumor, childhood astrocytomas, childhood brain stem glioma, childhood central nervous system atypical teratoid/rhabdoid tumor, childhood central nervous system embryonal tumors, childhood central nervous system germ cell tumors, childhood craniopharyngioma brain tumor, childhood ependymoma brain tumor, breast cancer, childhood bronchial tumors, carcinoid tumor, childhood carcinoid tumor, gastrointestinal carcinoid tumor, carcinoma of unknown primary, childhood carcinoma of unknown primary, childhood cardiac tumors, cervical cancer, childhood cervical cancer, childhood chordoma, chronic myeloproliferative disorders, colon cancer colorectal cancer, childhood colorectal cancer, extrahepatic bile duct cancer, ductal carcinoma in situ (DCIS), endometrial cancer, esophageal cancer, childhood esophageal cancer, childhood esthesioneuroblastoma, eye cancer, malignant fibrous histiocytoma of bone, gallbladder cancer, gastric (stomach) cancer, childhood gastric cancer, gastrointestinal stromal tumors (GIST), childhood gastrointestinal stromal tumors (GIST), childhood extracranial germ cell tumor, extragonadal germ cell tumor, gestational trophoblastic tumor, glioma, head and neck cancer, childhood head and neck cancer, hepatocellular cancer, hypopharyngeal cancer, kidney cancer, renal cell kidney cancer, Wilms tumor, childhood kidney tumors, Langerhans cell histiocytosis, laryngeal cancer, childhood laryngeal cancer, leukemia, acute lymphoblastic leukemia (ALL), acute myeloid leukemia (AML), chronic lymphocytic leukemia (CLL), chronic myelogenous leukemia (cml), hairy cell leukemia, lip cancer, liver cancer (primary), childhood liver cancer (primary), lobular carcinoma in situ (LCIS), lung cancer, non-small cell lung cancer, small cell lung cancer, lymphoma, AIDS-related lymphoma, burkitt lymphoma, cutaneous t-cell lymphoma. Hodgkin lymphoma, non-Hodgkin lymphoma, primary central nervous system lymphoma (CNS), melanoma, childhood melanoma, intraocular melanoma, Merkel cell carcinoma, malignant mesothelioma, childhood malignant mesothelioma, metastatic squamous neck cancer with occult primary, midline tract carcinoma involving NUT gene, mouth cancer, childhood multiple endocrine neoplasia syndromes, mycosis fungoides, myelodysplastic syndromes, myelodysplastic neoplasms, myeloproliferative neoplasms, multiple myeloma, nasal cavity cancer, nasopharyngeal cancer, childhood nasopharyngeal cancer, neuroblastoma, oral cancer, childhood oral cancer, oropharyngeal cancer, ovarian cancer, childhood ovarian cancer, epithelial ovarian cancer, low malignant potential tumor ovarian cancer, pancreatic cancer, childhood pancreatic cancer, pancreatic neuroendocrine tumors (islet cell tumors), childhood papillomatosis, paraganglioma, paranasal sinus cancer, parathyroid cancer, penile cancer, pharyngeal cancer, pheochromocytoma, pituitary tumor, plasma cell neoplasm, childhood pleuropulmonary blastoma, prostate cancer, rectal cancer, renal pelvis transitional cell cancer, retinoblastoma, salivary gland cancer, childhood salivary gland cancer. Ewing sarcoma family of tumors, Kaposi Sarcoma, osteosarcoma, rhabdomyosarcoma, childhood rhabdomyosarcoma, soft tissue sarcoma, uterine sarcoma, Sézary syndrome, childhood skin cancer, nonmelanoma skin cancer, small intestine cancer, squamous cell carcinoma, childhood squamous cell carcinoma, testicular cancer, childhood testicular cancer, throat cancer, thymoma and thymic carcinoma, childhood thymoma and thymic carcinoma, thyroid cancer, childhood thyroid cancer, ureter transitional cell cancer, urethral cancer, endometrial uterine cancer, vaginal cancer, vulvar cancer, and Waldenström macroglobulinemia.

The combination therapies described herein may be used together with other therapies such as radiation therapy. Chemotherapy and radiotherapy treatment regimens can comprise a finite number of cycles of on-drug therapy followed by off-drug therapy, or comprise a finite timeframe in which the chemotherapy or radiotherapy is administered. The protocols can be determined by clinical trials, drug labels, and clinical staff in conjunction with the subject to be treated. The number of cycles of a chemotherapy or radiotherapy or the total length of time of a chemotherapy or radiotherapy regimen can vary depending on the subject's response to the cancer therapy. A pharmaceutical agent described herein can be administered after the treatment regimen of chemotherapy or radiotherapy has been completed.

In some aspects, the combinations described herein can be utilized to treat a subject in need thereof. In some cases, the subject to be treated by methods and compositions disclosed herein can be a human subject. A subject to be treated by methods and compositions disclosed herein can be a non-human animal. Non-limiting examples of non-human animals can include a non-human primate, a livestock animal, a domestic pet, and a laboratory animal.

### Crystalline Forms

In an embodiment, the disclosure provides a crystalline solid form of voruciclib. In an embodiment, the disclosure provides a crystalline solid form of voruciclib free base. In an embodiment, the disclosure provides a crystalline solid form of a voruciclib salt. The disclosure provides polymorphs, for example crystal forms, of voruciclib. In some embodiments, the polymorphs include free base voruciclib. In some embodiments, the polymorphs include voruciclib salts including a counterion corresponding to an acid selected from 1,5-naphthalenedisulfonic acid, 1-hydroxy-2-naphthoic acid, benzenesulfonic acid, benzoic acid, dibenzoyl-L-tartaric acid, ethanesulfonic acid, gentisic acid, hydrobromic acid, hydrochloric acid, maleic acid, malonic acid, oxalic acid, ortho-phosphoric acid, sulfuric acid, p-toluenesulfonic acid, and the like.

Any crystalline form described herein can be characterized by X-ray diffraction. In some embodiments, X-ray diffraction refers to X-ray powder diffraction. In some embodiments, X-ray diffraction may be measured using transmission mode or reflection mode. In an embodiment, the X-ray diffraction pattern of any embodiments herein is measured in transmission mode. In an embodiment, the X-ray diffraction pattern of any embodiments herein is measured in reflection mode. It is known in the art that an X-ray powder diffraction pattern may be obtained which has one or more measurement errors depending on measurement conditions (such as equipment, sample preparation, or instrument used). In particular, it is generally known that intensities in an X-ray powder diffraction pattern may vary depending on measurement conditions and sample preparation. For example, persons skilled in the art of X-ray powder diffraction will realize that the relative intensities of peaks may vary according to the orientation of the sample under test and based on the type and settings of the instrument used. The skilled person will also realize that the position of reflections can be affected by the precise height at which the sample sits in the diffractometer, the sample's surface planarity, and the zero calibration of the diffractometer. Hence a person skilled in the art will appreciate that the diffraction pattern data presented herein is not to be construed as absolute and any crystalline form that provides a power diffraction pattern substantially the same as those disclosed herein fall within the scope of the present disclosure. For further information, see Jenkins and Snyder. Introduction to X-Ray Powder Diffractometry, John Wiley & Sons, 1996.

Different crystalline form may provide surprising advantages compared to non-crystalline forms, including improved thermodynamic stability, faster dissolution rate, improved performance in the stomach and gastric environment (including the avoidance of, or reduced, precipitation from solution upon a change to higher pH), improved exposure in mammals, and superior processability for formulation of drug into finished products suitable for patients.

In one embodiment, the disclosure provides a crystal form of voruciclib malonate, and/or a polymorph crystal form of voruciclib malonate (Mao1), characterized by an X-ray powder diffraction pattern including one or more peaks selected from:

| No | 2θ (°) | D (Å) | I (%) |
|---|---|---|---|
| 1 | 6.36 | 13.88 | 11 |
| 2 | 7.31 | 12.08 | 28 |
| 3 | 9.34 | 9.46 | 15 |
| 4 | 10.05 | 8.79 | 12 |
| 5 | 13.59 | 6.51 | 31 |
| 6 | 14.08 | 6.28 | 29 |
| 7 | 15.21 | 5.82 | 76 |
| 8 | 15.67 | 5.65 | 65 |
| 9 | 17.53 | 5.06 | 27 |
| 10 | 18.70 | 4.74 | 23 |
| 11 | 18.98 | 4.67 | 100 |
| 12 | 19.38 | 4.58 | 36 |
| 13 | 19.67 | 4.51 | 63 |
| 14 | 20.16 | 4.40 | 14 |
| 15 | 20.39 | 4.35 | 12 |
| 16 | 21.01 | 4.23 | 13 |
| 17 | 22.27 | 3.99 | 26 |
| 18 | 23.35 | 3.81 | 19 |
| 19 | 24.15 | 3.68 | 66 |
| 20 | 24.67 | 3.61 | 11 |
| 21 | 25.00 | 3.56 | 77 |
| 22 | 25.18 | 3.53 | 37 |
| 23 | 25.57 | 3.48 | 57 |
| 24 | 25.93 | 3.43 | 45 |
| 25 | 26.21 | 3.40 | 31 |
| 26 | 27.19 | 3.28 | 20 |
| 27 | 27.38 | 3.25 | 29 |

In some embodiments, each peak independently may include a variation of ±0.1°, ±0.2°, or ≤0.3°.

In one embodiment, the disclosure provides a crystal form of voruciclib oxalate, and/or a polymorph crystal form of voruciclib oxalate (Oxa1), characterized by an X-ray powder diffraction pattern including one or more peaks selected from:

| No | 2θ (°) | D (Å) | I (%) |
|---|---|---|---|
| 1 | 6.86 | 12.88 | 100 |
| 2 | 9.70 | 9.11 | 3 |
| 3 | 10.84 | 8.15 | 11 |
| 4 | 12.50 | 7.08 | 4 |
| 5 | 12.66 | 6.99 | 13 |
| 6 | 12.81 | 6.90 | 6 |
| 7 | 13.41 | 6.60 | 35 |
| 8 | 13.71 | 6.46 | 11 |
| 9 | 14.54 | 6.09 | 49 |
| 10 | 15.35 | 5.77 | 9 |
| 11 | 15.83 | 5.59 | 16 |
| 12 | 18.70 | 4.74 | 8 |
| 13 | 19.00 | 4.67 | 12 |
| 14 | 19.43 | 4.57 | 44 |
| 15 | 19.62 | 4.52 | 6 |
| 16 | 21.75 | 4.08 | 9 |
| 17 | 22.75 | 3.91 | 13 |
| 18 | 23.35 | 3.81 | 7 |
| 19 | 23.47 | 3.79 | 8 |
| 20 | 23.81 | 3.73 | 18 |
| 21 | 23.98 | 3.71 | 23 |
| 22 | 24.36 | 3.65 | 11 |
| 23 | 24.60 | 3.62 | 8 |
| 24 | 24.86 | 3.58 | 18 |
| 25 | 25.11 | 3.54 | 12 |
| 26 | 25.60 | 3.48 | 19 |
| 27 | 25.75 | 3.46 | 15 |
| 28 | 26.25 | 3.39 | 31 |

In some embodiments, each peak independently may include a variation of ±0.1°, ±0.2°, or ±0.3°.

In one embodiment, the disclosure provides a crystal form of voruciclib phosphate, and/or a polymorph crystal form of voruciclib phosphate (Pho1), characterized by an X-ray powder diffraction pattern including one or more peaks selected from:

| No | 2θ (°) | D (Å) | I (%) |
|---|---|---|---|
| 1 | 4.93 | 17.92 | 31 |
| 2 | 6.79 | 13.01 | 61 |
| 3 | 9.35 | 9.45 | 22 |
| 4 | 10.58 | 8.35 | 12 |
| 5 | 10.91 | 8.10 | 52 |
| 6 | 12.64 | 7.00 | 37 |
| 7 | 13.35 | 6.63 | 23 |
| 8 | 13.58 | 6.51 | 7 |
| 9 | 14.81 | 5.98 | 100 |
| 10 | 15.60 | 5.68 | 28 |
| 11 | 17.18 | 5.16 | 14 |
| 12 | 17.52 | 5.06 | 15 |
| 13 | 18.32 | 4.84 | 14 |
| 14 | 18.78 | 4.72 | 25 |
| 15 | 19.34 | 4.59 | 10 |
| 16 | 19.64 | 4.52 | 13 |
| 17 | 19.78 | 4.49 | 23 |
| 18 | 22.02 | 4.03 | 28 |
| 19 | 23.20 | 3.83 | 16 |
| 20 | 23.67 | 3.76 | 36 |
| 21 | 24.00 | 3.70 | 45 |
| 22 | 24.71 | 3.60 | 35 |
| 23 | 25.21 | 3.53 | 20 |
| 24 | 25.39 | 3.51 | 19 |
| 25 | 26.55 | 3.35 | 23 |
| 26 | 27.22 | 3.27 | 13 |
| 27 | 28.07 | 3.18 | 11 |
| 28 | 29.90 | 2.99 | 15 |

In some embodiments, each peak independently may include a variation of ±0.1°, ±0.2°, or ±0.3°.

In one embodiment, the disclosure provides a crystal form of voruciclib characterized by an X-ray powder diffraction pattern including one or more peaks selected from 7.30°±0.2°, 13.58°±0.2°, 14.06°±0.2°, 15.18°±0.2°, 15.66°±0.2°, 17.50°±0.2°, 18.94°±0.2°, 19.54°±0.2°, 22.22°±0.2°, 23.38°±0.2°, 24.10°±0.2°, 24.98°±0.2°, 25.94°±0.2°, 27.26°±0.2°, 28.50°±0.2°, and 32.82°±0.2° 2θ. In some embodiments, the X-ray diffraction pattern includes at least one peak, at least two peaks, at least three peaks, at least four peaks, at least five peaks, or the like, selected from the above group of peaks. In some embodiments, the crystal form includes voruciclib malonate. In some embodiments, the crystal form includes hydrated voruciclib malonate. In some embodiments, the crystal form includes anhydrous voruciclib malonate.

In one embodiment, the disclosure provides a crystal form of voruciclib characterized by an X-ray powder diffraction pattern including one or more peaks selected from 5.06° #0.2°, 6.42°±0.2°, 9.34°±0.2°, 10.14°±0.2°, 12.30°±0.2°, 13.66°±0.2°, 14.14°±0.2°, 15.82°±0.2°, 17.02°±0.2°, 19.74°±0.2°, 20.38°±0.2°, 21.82°±0.2°, 22.66°±0.2°, 24.62°±0.2°, 25.78°±0.2°, 26.58°±0.2°, 28.66°±0.2°, and 29.98°±0.2° 2θ. In some embodiments, the X-ray diffraction pattern includes at least one peak, at least two peaks, at least three peaks, at least four peaks, at least five peaks, or the like, selected from the above group of peaks. In some embodiments, the crystal form includes voruciclib dibenzoyl-tartrate. In some embodiments, the crystal form includes hydrated voruciclib dibenzoyl-tartrate. In some embodiments, the crystal form includes anhydrous voruciclib dibenzoyl-tartrate.

In one embodiment, the disclosure provides a crystal form of voruciclib characterized by an X-ray powder diffraction pattern including one or more peaks selected from 4.94°±0.2°, 6.78°±0.2°, 9.34°±0.2°, 10.94°±0.2°, 12.70°±0.2°, 13.38°±0.2°, 14.90°±0.2°, 15.66°±0.2°, 17.54°±0.2°, 18.82°±0.2°, 22.02°±0.2°, 23.98°±0.2°, 24.78°±0.2°, 25.30°±0.2°, 26.66°±0.2°, and 29.98°±0.2° 2θ. In some embodiments, the X-ray diffraction pattern includes at least one peak, at least two peaks, at least three peaks, at least four peaks, at least five peaks, or the like, selected from the above group of peaks. In some embodiments, the crystal form includes voruciclib phosphate. In some embodiments, the crystal form includes hydrated voruciclib phosphate. In some embodiments, the crystal form includes anhydrous voruciclib phosphate.

In one embodiment, the disclosure provides a crystal form of voruciclib characterized by an X-ray powder diffraction pattern including one or more peaks selected from 6.86°±0.2°, 12.66°±0.2°, 13.58°±0.2°, 14.74°±0.2°, 15.98°±0.2°, 19.38°±0.2°, 23.94°±0.2º, 24.78°±0.2°, and 25.94°±0.2° 2θ. In some embodiments, the X-ray diffraction pattern includes at least one peak, at least two peaks, at least three peaks, at least four peaks, at least five peaks, or the like, selected from the above group of peaks. In some embodiments, the crystal form includes voruciclib oxalate. In some embodiments, the crystal form includes hydrated voruciclib oxalate. In some embodiments, the crystal form includes anhydrous voruciclib oxalate.

In one embodiment, the disclosure provides a crystal form of voruciclib characterized by an X-ray powder diffraction pattern including one or more peaks selected from 9.02°±0.2°, 10.50°±0.2°, 11.06°±0.2°, 12.30°±0.2°, 12.82°±0.2°, 13.90°±0.2°, 14.82°±0.2°, 15.30°±0.2°, 15.94°±0.2°, 17.26°±0.2°, 19.34°±0.2°, 20.62°±0.2°, 22.18°±0.2°, 22.86°±0.2°, 24.58°±0.2°, 25.42°±0.2°, 25.86°±0.2°, 27.38°±0.2°, and 28.66°±0.2° 20. In some embodiments, the X-ray diffraction pattern includes at least one peak, at least two peaks, at least three peaks, at least four peaks, at least five peaks, or the like, selected from the above group of peaks. In some embodiments, the crystal form includes voruciclib napadisylate. In some embodiments, the crystal form includes hydrated voruciclib napadisylate. In some embodiments, the crystal form includes anhydrous voruciclib napadisy late.

### Pharmaceutical Compositions

In an embodiment, the disclosure provides a pharmaceutical composition comprising a crystalline form of the voruciclib free base. In an embodiment, the disclosure provides a pharmaceutical composition comprising a crystalline form of a voruciclib salt. The pharmaceutical compositions are typically formulated to provide a therapeutically effective amount of a solid form of voruciclib as the active ingredient, or a pharmaceutically acceptable salt, ester, prodrug, solvate, hydrate or derivative thereof. Where desired, the pharmaceutical compositions contains a pharmaceutically acceptable salt thereof, and one or more pharmaceutically acceptable excipients, carriers, including inert solid diluents and fillers, diluents, permeation enhancers, solubilizers, or adjuvants. The pharmaceutical compositions may also contain an acidulant, as described herein.

In some embodiments, the concentration of a solid form of voruciclib, including any voruciclib free base polymorph described herein, or any voruciclib salt polymorph described herein, provided in the pharmaceutical compositions of the disclosure, is independently less than, for example, 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, or 0.001% w/w; w/v, or v/v, relative to the total mass or volume of the pharmaceutical composition. In an embodiment, the solid form of voruciclib is selected from voruciclib malonate, voruciclib dibenzoyl-tartrate, voruciclib phosphate, voruciclib oxalate, and voruciclib napadisylate, each as described herein.

In some embodiments, the concentration of a solid form of voruciclib, including any voruciclib free base polymorph described herein, or any voruciclib salt polymorph described herein, provided in the pharmaceutical compositions of the disclosure is independently greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, or 0.001% w/w; w/v; or v/v, relative to the total mass or volume of the pharmaceutical composition. In an embodiment, the solid form of voruciclib is selected from voruciclib malonate, voruciclib dibenzoyl-tartrate, voruciclib phosphate, voruciclib oxalate, and voruciclib napadisylate, each as described herein.

In some embodiments, the concentration of a solid form of voruciclib, including any voruciclib free base polymorph described herein, or any voruciclib salt polymorph described herein, is independently in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12% or approximately 1% to approximately 10% w/w, w/v or v/v, relative to the total mass or volume of the pharmaceutical composition. In an embodiment, the solid form of voruciclib is selected from voruciclib malonate, voruciclib dibenzoyl-tartrate, voruciclib phosphate, voruciclib oxalate, and voruciclib napadisylate, each as described herein.

In some embodiments, the concentration of voruciclib, including any voruciclib free base polymorph described herein, or any voruciclib salt polymorph described herein, is independently in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v, or v/v, relative to the total mass or volume of the pharmaceutical composition. In an embodiment, the solid form of voruciclib is selected from voruciclib malonate, voruciclib dibenzoyl-tartrate, voruciclib phosphate, voruciclib oxalate, and voruciclib napadisylate, each as described herein.

In some embodiments, the amount of voruciclib, including any voruciclib free base polymorph described herein, or any voruciclib salt polymorph described herein, is independently equal to or less than 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g or 0.0001 g. In an embodiment, the solid form of

In some embodiments, the amount of a solid form of voruciclib, including any voruciclib free base polymorph described herein, or any voruciclib salt polymorph described herein, is independently more than 0.0001 g, 0.0002 g, 0.0003 g, 0.0004 g, 0.0005 g, 0.0006 g, 0.0007 g. 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g. 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, or 3 g. In an embodiment, the solid form of voruciclib is selected from voruciclib malonate, voruciclib dibenzoyl-tartrate, voruciclib phosphate, voruciclib oxalate, and voruciclib napadisylate, each as described herein.

Each of the solid forms of voruciclib, including any voruciclib free base polymorph described herein, or any voruciclib salt polymorph described herein, is effective over a wide dosage range. For example, in the treatment of adult humans, dosages independently range from 0.01 to 1000 mg, from 0.5 to 100 mg, from 1 to 50 mg per day, from 2 to 40 mg per day, and from 5 to 25 mg per day are examples of dosages that may be used. The exact dosage will depend upon the route of administration, the form in which the compound is administered, the gender and age of the subject to be treated, the body weight of the subject to be treated, and the preference and experience of the attending physician. In an embodiment, the solid form of voruciclib is selected from voruciclib malonate, voruciclib dibenzoyl-tartrate, voruciclib phosphate, voruciclib oxalate, and voruciclib napadisylate, each as described herein.

In selected embodiments, the disclosure provides a pharmaceutical composition for oral administration containing voruciclib, including any voruciclib free base polymorph described herein, or any voruciclib salt polymorph described herein, and a pharmaceutical excipient suitable for oral administration. In an embodiment, the solid form of voruciclib is selected from voruciclib malonate, voruciclib dibenzoyl-tartrate, voruciclib phosphate, voruciclib oxalate, and voruciclib napadisylate, each as described herein.

In selected embodiments, the disclosure provides a solid pharmaceutical composition for oral administration containing: (i) an effective amount of voruciclib, including any voruciclib free base polymorph described herein, or any voruciclib salt polymorph described herein, and (ii) a pharmaceutical excipient suitable for oral administration. In selected embodiments, the composition further contains (iii) an effective amount of another active pharmaceutical ingredient. In an embodiment, the solid form of voruciclib is selected from voruciclib malonate, voruciclib dibenzoyl-tartrate, voruciclib phosphate, voruciclib oxalate, and voruciclib napadisy late, each as described herein.

In selected embodiments, the pharmaceutical composition may be a liquid pharmaceutical composition suitable for oral consumption. Pharmaceutical compositions of the disclosure suitable for oral administration can be presented as discrete dosage forms, such as capsules, sachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil emulsion. Pharmaceutical compositions of the disclosure also include powder for reconstitution, powders for oral consumptions, bottles (such as powder or liquid in bottle), orally dissolving films, lozenges, pastes, tubes, gums, and packs. Such dosage forms can be prepared by any of the methods of pharmacy, but all methods include the step of bringing the active ingredient(s) into association with the carrier, which constitutes one or more necessary ingredients. In general, the compositions are prepared by uniformly and intimately admixing the active ingredient(s) with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

The disclosure further encompasses anhydrous pharmaceutical compositions and dosage forms since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the disclosure can be prepared using anhydrous or low moisture containing ingredients and low moisture or low humidity conditions. Pharmaceutical compositions and dosage forms of the disclosure which contain lactose can be made anhydrous if substantial contact with moisture and/or humidity during manufacturing, packaging, and/or storage is expected. An anhydrous pharmaceutical composition may be prepared and stored such that its anhydrous nature is maintained.

Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

Each of the solid forms of voruciclib, including any voruciclib free base polymorph described herein, or any voruciclib salt polymorph described herein, can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, sodium cross carmelose, magnesium stearate, diluents, granulating agents, lubricants, glidants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the disclosure to provide tablets that disintegrate when exposed to an aqueous environment. Too much of a disintegrant may produce tablets which disintegrate in the bottle. Too little may be insufficient for disintegration to occur, thus altering the rate and extent of release of the active ingredients from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the disclosure include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the disclosure include, but are not limited to, calcium stearate, magnesium stearate, mineral oil, light mineral oil, glycerin, sorbitol, mannitol, polyethylene glycol, other glycols, stearic acid, sodium stearyl fumarate, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethylaureate, agar, or mixtures thereof. Additional lubricants include, for example, a syloid silica gel, a coagulated aerosol of synthetic silica, silicified microcrystalline cellulose, or mixtures thereof. A lubricant can optionally be added, in an amount of less than about 1 weight percent of the pharmaceutical composition.

When aqueous suspensions and/or elixirs are desired for oral administration, the essential active ingredient therein may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactants which can be used to form pharmaceutical compositions and dosage forms of the disclosure include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

An empirical parameter used to characterize the relative hydrophilicity and hydrophobicity of non-ionic amphiphilic compounds is the hydrophilic-lipophilic balance (“HLB” value). A suitable hydrophilic surfactant may generally have an HLB value of at least 10, while suitable lipophilic surfactants may generally have an HLB value of or less than about 10. Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyllactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acyllactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols;

polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10-oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols, glycerol fatty acid esters, acetylated glycerol fatty acid esters, lower alcohol fatty acids esters, propylene glycol fatty acid esters, sorbitan fatty acid esters, polyethylene glycol sorbitan fatty acid esters, sterols and sterol derivatives, polyoxyethylated sterols and sterol derivatives, polyethylene glycol alkyl ethers, sugar esters, sugar ethers, lactic acid derivatives of mono- and di-glycerides, and hydrophobic transesterification products of a polyol with at least one member of the group consisting of glycerides, vegetable oils, hydrogenated vegetable oils, fatty acids and sterols, oil-soluble vitamins/vitamin derivatives, and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In an embodiment, the composition may include a solubilizer to ensure good solubilization and/or dissolution of the compound of the present disclosure and to minimize precipitation of the compound of the present disclosure. This can be especially important for compositions for non-oral use—e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, xylitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxy alkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, .epsilon.-caprolactone and isomers thereof, δ-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a patient using conventional techniques, such as distillation or evaporation. Thus, if present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1%, or even less. Typically, the solubilizer may be present in an amount of about 1% to about 100%, more typically about 5% to about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the pharmaceutical composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals and alkaline earth metals. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, and uric acid.

### Dosages and Dosing Regimens

The amounts of the solid form of voruciclib, including any voruciclib free base polymorph described herein, or any voruciclib salt polymorph described herein, administered will be dependent on the mammal being treated, the severity of the disorder or condition, the rate of administration, the disposition of the compounds and the discretion of the prescribing physician. However, an effective dosage is in the range of about 0.001 to about 100 mg per kg body weight per day, such as about 1 to about 35 mg/kg/day, in single or divided doses. For a 70 kg human, this would amount to about 0.05 to 7 g/day, such as about 0.05 to about 2.5 g/day. In some instances, dosage levels below the lower limit of the aforesaid range may be more than adequate, while in other cases still larger doses may be employed without causing any harmful side effect, for example by dividing such larger doses into several small doses for administration throughout the day.

In selected embodiments, a solid form of voruciclib, including any voruciclib free base polymorph described herein, or any voruciclib salt polymorph described herein, is administered in a single dose. Typically, such administration will be by injection, for example by intravenous injection, in order to introduce the active pharmaceutical ingredients quickly. However, other routes may be used as appropriate. A single dose of a solid form of voruciclib, including any voruciclib free base polymorph described herein, or any voruciclib salt polymorph described herein, may also be used for treatment of an acute condition.

In selected embodiments, a solid form of voruciclib, including any voruciclib free base polymorph described herein, or any voruciclib salt polymorph described herein, is administered in multiple doses. Dosing may be about once, twice, three times, four times, five times, six times, or more than six times per day. Dosing may be about once a month, once every two weeks, once a week, or once every other day. In other embodiments, a solid form of voruciclib, including any voruciclib free base polymorph described herein, or any voruciclib salt polymorph described herein, is administered about once per day to about 6 times per day. In another embodiment the administration of the solid forms of voruciclib, including any voruciclib free base polymorph described herein, or any voruciclib salt polymorph described herein, continues for less than about 7 days. In yet another embodiment the administration continues for more than about 6, 10, 14, 28 days, two months, six months, or one year. In some cases, continuous dosing is achieved and maintained as long as necessary. In an embodiment, the solid form of voruciclib is selected from voruciclib malonate, voruciclib dibenzoyl-tartrate, voruciclib phosphate, voruciclib oxalate, and voruciclib napadisylate, each as described herein.

Administration of the active pharmaceutical ingredients of the disclosure may continue as long as necessary. In selected embodiments, a solid form of voruciclib, including any voruciclib free base polymorph described herein, or any voruciclib salt polymorph described herein, is administered for more than 1, 2, 3, 4, 5, 6, 7, 14, or 28 days. In some embodiments, the solid forms of voruciclib, including any voruciclib free base polymorph described herein, or any voruciclib salt polymorph described herein, are administered for less than 28, 14, 7, 6, 5, 4, 3, 2, or 1 day. In selected embodiments, a solid form of voruciclib, including any voruciclib free base polymorph described herein, or any voruciclib salt polymorph described herein, is administered chronically on an ongoing basis—e.g., for the treatment of chronic effects. In an embodiment, the solid form of voruciclib, in any of the foregoing embodiments is selected from voruciclib malonate, voruciclib dibenzoyl-tartrate, voruciclib phosphate, voruciclib oxalate, and voruciclib napadisylate, each as described herein.

In some embodiments, an effective dosage of voruciclib, including any voruciclib free base polymorph described herein, or any voruciclib salt polymorph described herein, is in the range of about 1 mg to about 500 mg, about 10 mg to about 300 mg, about 20 mg to about 250 mg, about 25 mg to about 200 mg, about 10 mg to about 200 mg, about 20 mg to about 150 mg, about 30 mg to about 120 mg, about 10 mg to about 90 mg, about 20 mg to about 80 mg, about 30 mg to about 70 mg, about 40 mg to about 60 mg, about 45 mg to about 55 mg, about 48 mg to about 52 mg, about 50 mg to about 150 mg, about 60 mg to about 140 mg, about 70 mg to about 130 mg, about 80 mg to about 120 mg, about 90 mg to about 110 mg, about 95 mg to about 105 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 202 mg. In some embodiments, an effective dosage of a solid form of voruciclib, including any voruciclib free base polymorph described herein, or any voruciclib salt polymorph described herein, is about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, or about 500 mg, In some embodiments, an effective dosage of a solid form of voruciclib, including any voruciclib free base polymorph described herein, or any voruciclib salt polymorph described herein, is 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, or 500 mg. In an embodiment, the solid form of voruciclib in any of the foregoing embodiments is selected from voruciclib malonate, voruciclib dibenzoyl-tartrate, voruciclib phosphate, voruciclib oxalate, and voruciclib napadisylate, each as described herein.

In some embodiments, an effective dosage of voruciclib, including any voruciclib free base polymorph described herein, or any voruciclib salt polymorph described herein, is in the range of about 0.01 mg/kg to about 4.3 mg/kg, about 0.15 mg/kg to about 3.6 mg/kg, about 0.3 mg/kg to about 3.2 mg/kg, about 0.35 mg/kg to about 2.85 mg/kg, about 0.15 mg/kg to about 2.85 mg/kg, about 0.3 mg to about 2.15 mg/kg, about 0.45 mg/kg to about 1.7 mg/kg, about 0.15 mg/kg to about 1.3 mg/kg, about 0.3 mg/kg to about 1.15 mg/kg, about 0.45 mg/kg to about 1 mg/kg, about 0.55 mg/kg to about 0.85 mg/kg, about 0.65 mg/kg to about 0.8 mg/kg, about 0.7 mg/kg to about 0.75 mg/kg, about 0.7 mg/kg to about 2.15 mg/kg, about 0.85 mg/kg to about 2 mg/kg, about 1 mg/kg to about 1.85 mg/kg, about 1.15 mg/kg to about 1.7 mg/kg, about 1.3 mg/kg mg to about 1.6 mg/kg, about 1.35 mg/kg to about 1.5 mg/kg, about 2.15 mg/kg to about 3.6 mg/kg, about 2.3 mg/kg to about 3.4 mg/kg, about 2.4 mg/kg to about 3.3 mg/kg, about 2.6 mg/kg to about 3.15 mg/kg, about 2.7 mg/kg to about 3 mg/kg, about 2.8 mg/kg to about 3 mg/kg, or about 2.85 mg/kg to about 2.95 mg/kg. In some embodiments, an effective dosage of a solid form of voruciclib, including any voruciclib free base polymorph described herein, or any voruciclib salt polymorph described herein, is about 0.35 mg/kg, about 0.7 mg/kg, about 1 mg/kg, about 1.4 mg/kg, about 1.8 mg/kg, about 2.1 mg/kg, about 2.5 mg/kg, about 2.85 mg/kg, about 3.2 mg/kg, or about 3.6 mg/kg. In an embodiment, the solid form of voruciclib in any of the foregoing embodiments is selected from voruciclib malonate, voruciclib dibenzoyl-tartrate, voruciclib phosphate, voruciclib oxalate, and voruciclib napadisylate, each as described herein.

In some embodiments, a solid form of voruciclib, including any voruciclib free base polymorph described herein, or any voruciclib salt polymorph described herein, is administered at a dosage of 10 to 400 mg once daily (QD), including a dosage of 5 mg, 10 mg. 12.5 mg, 25 mg, 50 mg, 75 mg, 100 mg, 150 mg, 175 mg, 200 mg, 225 mg, 250 mg, 275 mg, 300 mg, 325 mg, 350 mg, 375 mg, 400 mg, 425 mg, 450 mg, 475 mg, and 500 mg once daily (QD). In an embodiment, the solid form of voruciclib in any of the foregoing embodiments is selected from voruciclib malonate, voruciclib dibenzoyl-tartrate, voruciclib phosphate, voruciclib oxalate, and voruciclib napadisylate, each as described herein.

In some embodiments, a solid form of voruciclib, including any voruciclib free base polymorph described herein, or any voruciclib salt polymorph described herein, is administered at a dosage of 10 to 400 mg BID, including a dosage of 5 mg, 10 mg, 12.5 mg, 25 mg, 50 mg, 75 mg, 100 mg, 150 mg, 175 mg, 200 mg, 225 mg, 250 mg, 275 mg, 300 mg, 325 mg, 350 mg, 375 mg, 400 mg, 425 mg, 450 mg, 475 mg, and 500 mg BID. In an embodiment, the solid form of voruciclib in any of the foregoing embodiments is selected from voruciclib malonate, voruciclib dibenzoyl-tartrate, voruciclib phosphate, voruciclib oxalate, and voruciclib napadisylate, each as described herein.

In some embodiments, a solid form of voruciclib, including any voruciclib free base polymorph described herein, or any voruciclib salt polymorph described herein, is administered at a dosage of 10 to 400 mg TID, including a dosage of 5 mg, 10 mg, 12.5 mg, 25 mg, 50 mg, 75 mg, 100 mg, 150 mg, 175 mg, 200 mg, 225 mg, 250 mg, 275 mg, 300 mg, 325 mg, 350 mg, 375 mg, 400 mg, 425 mg, 450 mg, 475 mg, and 500 mg TID. In an embodiment, the solid form of voruciclib in any of the foregoing embodiments is selected from voruciclib malonate, voruciclib dibenzoyl-tartrate, voruciclib phosphate, voruciclib oxalate, and voruciclib napadisylate, each as described herein.

An effective amount of a solid form of voruciclib, including any voruciclib free base polymorph described herein, or any voruciclib salt polymorph described herein, may be administered in either single or multiple doses by any of the accepted modes of administration of active pharmaceutical ingredients having similar utilities, including rectal, buccal, intranasal and transdermal routes, by intra-arterial injection, intravenously, intraperitoneally, parenterally, intramuscularly, subcutaneously, orally, topically, or as an inhalant.

### Pharmaceutical Compositions for Overcoming the Effects of Acid Reducing Agents

The compositions and methods described herein can be used to overcome the effects of acid reducing agents. Acid-reducing agents can greatly limit the exposure of weakly acidic drugs in mammals. Smelick, et al., Mol. Pharmaceutics 2013, 10. 4055-4062. Acid reducing agents include proton pump inhibitors, such as omeprazole, esomeprazole, lansoprazole, dexlansoprazole, pantoprazole, rabeprazole, and ilaprazole; H₂ receptor antagonists, such as cimetidine, ranitidine, and famotidine; and antacids such as bicarbonates, carbonates, and hydroxides of aluminum, calcium, magnesium, potassium, and sodium, as well as mixtures of antacids with agents targeting mechanisms of gastric secretion. Overcoming the effects of acid reducing agents is a significant issue in the treatment of patients with cancer, inflammatory diseases, immune diseases, and autoimmune diseases, since these patients are commonly co-administered acid reducing agents for gastric irritation that often accompanies their conditions, because acid reducing agents are some of the most commonly prescribed medications in North America and Western Europe. Most recently approved oral cancer therapeutics have pH-dependent solubility and thus a potential drug-drug interaction with regards to acid reducing agents. In cancer patients, it is estimated that 20-33% of all patients are using some form of acid-reducing agent. In particular cancers, such as pancreatic cancer or gastrointestinal cancers, acid reducing agent use is as high as 60-80% of patients. Smelick, et al., Mol. Pharmaceutics 2013, 10. 4055-4062.

In an embodiment, a pharmaceutical composition comprises voruciclib, including any voruciclib free base polymorph described herein, or any voruciclib salt polymorph described herein, and an acidulant. In an embodiment, a pharmaceutical composition comprises voruciclib, including any voruciclib free base polymorph described herein, or any voruciclib salt polymorph described herein, and an acidulant selected from the group consisting of fumaric acid, tartaric acid, ascorbic acid, alginic acid, sodium alginate, potassium alginate, and Carbopol 971P (carboxypolymethylene). In an embodiment, a pharmaceutical composition comprises voruciclib, including any voruciclib free base polymorph described herein, or any voruciclib salt polymorph described herein, and an acidulant selected from the group consisting of fumaric acid, succinic acid, D-tartaric acid, L-tartaric acid, racemic tartaric acid, ascorbic acid, isoascorbic acid (also known as erythorbic acid and D-araboascorbic acid), alginic acid, Protacid F 120 NM, Protacid AR 1112 (also known as Kelacid NF), Carbomer 941 (polyacrylic acid), and Carbopol 971P (carboxypolymethylene). In an embodiment, the solid form of voruciclib in any of the foregoing embodiments is selected from voruciclib malonate, voruciclib dibenzoyl-tartrate, voruciclib phosphate, voruciclib oxalate, and voruciclib napadisylate, each as described herein. In an embodiment, the acidulant is extragranular. In an embodiment, the acidulant is intragranular.

Alginic acid is a polysaccharide copolymer, β-D-mannuronic acid (M) and α-L-guluronic acid (G) linked by 1-4 glycosidic bonds. In an embodiment, a pharmaceutical composition comprises voruciclib, including any voruciclib free base polymorph described herein, or any voruciclib salt polymorph described herein, and an acidulant that is an alginic acid or salt thereof, wherein the alginic acid or salt thereof exhibits an M/G ratio selected from the group consisting of between 0.1 and 0.5, between 0.2 and 0.6, between 0.3 and 0.7, between 0.4 and 0.8, between 0.5 and 0.9, between 0.6 and 1.0, between 0.7 and 1.1, between 0.8 and 1.2, between 0.9 and 1.3, between 1.0 and 1.4, between 1.1 and 1.5, between 1.2 and 1.6, between 1.3 and 1.7, between 1.4 and 1.8, between 1.5 and 1.9, between 1.6 and 2.0, between 1.7 and 2.1, between 1.8 and 2.2, between 1.9 and 2.3, between 2.0 and 2.4, and between 2.1 and 2.5. In an embodiment, a pharmaceutical composition comprises voruciclib, including any voruciclib free base polymorph described herein, or any voruciclib salt polymorph described herein, and an acidulant that is an alginic acid or salt thereof, wherein the alginic acid or salt thereof exhibits an M/G ratio selected from the group consisting of less than 0.5, less than 1.0, less than 1.5, less than 2.0, and less than 2.5. In an embodiment, a pharmaceutical composition comprises voruciclib, including any voruciclib free base polymorph described herein, or any voruciclib salt polymorph described herein, and an acidulant that is an alginic acid or salt thereof, wherein the alginic acid or salt thereof exhibits an M/G ratio selected from the group consisting of greater than 0.5, greater than 1.0, greater than 1.5, greater than 2.0, and greater than 2.5. In an embodiment, a pharmaceutical composition comprises voruciclib, including any voruciclib free base polymorph described herein, or any voruciclib salt polymorph described herein, and an acidulant that is an alginic acid or salt thereof, wherein the alginic acid or salt thereof exhibits an M/G ratio selected from the group consisting of 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5. In an embodiment, the solid form of voruciclib in any of the foregoing embodiments is selected from voruciclib malonate, voruciclib dibenzoyl-tartrate, voruciclib phosphate, voruciclib oxalate, and voruciclib napadisy late, each as described herein.

M/G ratio, as well as the fraction of M and G groups, the fractions of MM and GG “diads,” the fractions of “triads” (e.g., MGG), and the fractions of larger sequences of M and G groups, may be determined by methods known to those of ordinary skill in the art, including nuclear magnetic resonance (NMR) spectroscopy (with or without digestion) and mass spectrometry. Larsen, et al., Carbohydr. Res., 2003, 338. 2325-2336.

In an embodiment, a pharmaceutical composition comprises voruciclib, including any voruciclib free base polymorph described herein, or any voruciclib salt polymorph described herein, and an acidulant in a concentration (% mass) selected from the group consisting of between 1% and 5%, between 5% and 10%, between 10% and 15%, between 15% and 20%, between 20% and 25%, between 25% and 30%, and between 30% and 35%. In an embodiment, a pharmaceutical composition comprises voruciclib, including any voruciclib free base polymorph described herein, or any voruciclib salt polymorph described herein, and an acidulant in a concentration (% mass) selected from the group consisting of between 1% and 5%, between 5% and 10%, between 10% and 15%, between 15% and 20%, between 20% and 25%, between 25% and 30%, and between 30% and 35%, wherein the acidulant is selected from the group consisting of fumaric acid, succinic acid, D-tartaric acid, L-tartaric acid, racemic tartaric acid, ascorbic acid, isoascorbic acid (also known as erythorbic acid and D-araboascorbic acid), alginic acid, sodium alginate, potassium alginate, Protacid F 120 NM, Protacid AR 1112 (also known as Kelacid NF), and Carbopol 971P (carboxypolymethylene). In an embodiment, the solid form of voruciclib in any of the foregoing embodiments is selected from voruciclib malonate, voruciclib dibenzoyl-tartrate, voruciclib phosphate, voruciclib oxalate, and voruciclib napadisylate, each as described herein.

In an embodiment, a pharmaceutical composition comprises voruciclib, including any voruciclib free base polymorph described herein, or any voruciclib salt polymorph described herein, and an acidulant in a concentration (% mass) selected from the group consisting of less than 1%, less than 5%, less than 10%, less than 15%, less than 20%, less than 25%, less than 30%, and less than 35%. In an embodiment, a pharmaceutical composition comprises voruciclib, including any voruciclib free base polymorph described herein, or any voruciclib salt polymorph described herein, and an acidulant in a concentration (% mass) selected from the group consisting of less than 1%, less than 5%, less than 10%, less than 15%, less than 20%, less than 25%, less than 30%, and less than 35%, wherein the acidulant is selected from the group consisting of fumaric acid, succinic acid, D-tartaric acid, L-tartaric acid, racemic tartaric acid, ascorbic acid, isoascorbic acid (also known as erythorbic acid and D-araboascorbic acid), alginic acid, sodium alginate, potassium alginate, Protacid F 120 NM, Protacid AR 1112 (also known as Kelacid NF), and Carbopol 971P (carboxypolymethylene). In an embodiment, the solid form of voruciclib in any of the foregoing embodiments is selected from voruciclib malonate, voruciclib dibenzoyl-tartrate, voruciclib phosphate, voruciclib oxalate, and voruciclib napadisylate, each as described herein.

In an embodiment, a pharmaceutical composition comprises voruciclib, including any voruciclib free base polymorph described herein, or any voruciclib salt polymorph described herein, and an acidulant in a concentration (% mass) selected from the group consisting of greater than 1%, greater than 5%, greater than 10%, greater than 15%, greater than 20%, greater than 25%, greater than 30%, and greater than 35%. In an embodiment, a pharmaceutical composition comprises voruciclib, including any voruciclib free base polymorph described herein, or any voruciclib salt polymorph described herein, and an acidulant in a concentration (% mass) selected from the group consisting of greater than 1%, greater than 5%, greater than 10%, greater than 15%, greater than 20%, greater than 25%, greater than 30%, and greater than 35%, wherein the acidulant is selected from the group consisting of fumaric acid, succinic acid, D-tartaric acid, L-tartaric acid, racemic tartaric acid, ascorbic acid, isoascorbic acid (also known as erythorbic acid and D-araboascorbic acid), alginic acid, sodium alginate, potassium alginate, Protacid F 120 NM, Protacid AR 1112 (also known as Kelacid NF), and Carbopol 971P (carboxypolymethylene). In an embodiment, the solid form of voruciclib in any of the foregoing embodiments is selected from voruciclib malonate, voruciclib dibenzoyl-tartrate, voruciclib phosphate, voruciclib oxalate, and voruciclib napadisylate, each as described herein.

In an embodiment, a pharmaceutical composition comprises voruciclib, including any voruciclib free base polymorph described herein, or any voruciclib salt polymorph described herein, and an acidulant in a concentration (% mass) selected from the group consisting of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, and about 40%. In an embodiment, a pharmaceutical composition comprises voruciclib, including any voruciclib free base polymorph described herein, or any voruciclib salt polymorph described herein, and an acidulant in a concentration (% mass) selected from the group consisting of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, and about 40%, wherein the acidulant is selected from the group consisting of fumaric acid, succinic acid, D-tartaric acid, L-tartaric acid, racemic tartaric acid, ascorbic acid, isoascorbic acid (also known as erythorbic acid and D-araboascorbic acid), alginic acid, sodium alginate, potassium alginate, Protacid F 120 NM, Protacid AR 1112 (also known as Kelacid NF), and Carbopol 971P (carboxypolymethylene). In an embodiment, the solid form of voruciclib in any of the foregoing embodiments is selected from voruciclib malonate, voruciclib dibenzoyl-tartrate, voruciclib phosphate, voruciclib oxalate, and voruciclib napadisylate, each as described herein.

In an embodiment, a pharmaceutical composition comprises voruciclib, including any voruciclib free base polymorph described herein, or any voruciclib salt polymorph described herein, and an extragranular acidulant, wherein the extragranular acidulant is selected from the group consisting of fumaric acid, succinic acid, D-tartaric acid, L-tartaric acid, racemic tartaric acid, ascorbic acid, isoascorbic acid (also known as erythorbic acid and D-araboascorbic acid), alginic acid, sodium alginate, potassium alginate, Protacid F 120 NM, Protacid AR 1112 (also known as Kelacid NF), and Carbopol 971P (carboxypolymethylene), and combinations thereof. In an embodiment, a pharmaceutical composition comprises voruciclib, including any voruciclib free base polymorph described herein, or any voruciclib salt polymorph described herein, and an extragranular acidulant, wherein the extragranular acidulant is fumaric acid at a concentration of between about 15% to about 33% by weight. In an embodiment, a pharmaceutical composition comprises voruciclib, including any voruciclib free base polymorph described herein, or any voruciclib salt polymorph described herein, and an extragranular acidulant, wherein the extragranular acidulant is alginic acid or a salt thereof (such as sodium alginate or potassium alginate) at a concentration of between about 5% to about 33% by weight. In an embodiment, a pharmaceutical composition comprises voruciclib, including any voruciclib free base polymorph described herein, or any voruciclib salt polymorph described herein, and an extragranular acidulant, wherein the extragranular acidulant is L-tartaric acid at a concentration of between about 25% to about 33% by weight. In an embodiment, a pharmaceutical composition comprises voruciclib, including any voruciclib free base polymorph described herein, or any voruciclib salt polymorph described herein, and an extragranular acidulant, wherein the extragranular acidulant is ascorbic acid at a concentration of between about 20% to about 50% by weight and Carbopol 971P (carboxypolymethylene) at a concentration of between about 2.5% to about 10% by weight. In an embodiment, a pharmaceutical composition comprises voruciclib, including any voruciclib free base polymorph described herein, or any voruciclib salt polymorph described herein, and an extragranular acidulant, wherein the extragranular acidulant is fumaric acid at a concentration of between about 5% to about 15% by weight and alginic acid or a salt thereof at a concentration of about 15% to about 33% by weight. In an embodiment, a pharmaceutical composition comprises voruciclib, including any voruciclib free base polymorph described herein, or any voruciclib salt polymorph described herein, and an extragranular acidulant, wherein the extragranular acidulant is L-tartaric acid at a concentration of between about 5% to 15% by weight and alginic acid at a concentration of between about 15% to about 33% by weight.

In an embodiment, a pharmaceutical composition comprises voruciclib, including any voruciclib free base polymorph described herein, or any voruciclib salt polymorph described herein, and an acidulant, wherein the acidulant is selected from the group consisting of fumaric acid, maleic acid, phosphoric acid, L-tartaric acid, citric acid, gentisic acid, oxalic acid, and sulfuric acid. In an embodiment, a pharmaceutical composition comprises voruciclib, including any voruciclib free base polymorph described herein, or any voruciclib salt polymorph described herein, and an acidulant, wherein the acidulant is selected from the group consisting of fumaric acid, maleic acid, phosphoric acid, L-tartaric acid, citric acid, gentisic acid, oxalic acid, and sulfuric acid, and wherein the acidulant is a salt counterion included in any crystalline form described herein.

In an embodiment, in addition to an acidulant, a pharmaceutical composition includes an excipient to prolong the exposure of voruciclib, including any voruciclib free base polymorph described herein, or any voruciclib salt polymorph described herein, to the acidic microenvironment. In an embodiment, this excipient is a polymer of natural, synthetic or semisynthetic origins. The polymer may contain acidic, anionic, or non-ionic monomers, oligomers or polymers or a mixture of acidic, anionic and non-ionic monomers or copolymers. In one version the excipient is selected from the group consisting of hydroxypropylmethylcellulose, low substituted hydroxypropylcellulose, hydroxypropylcellulose, tocopherol polyethyleneoxide succinate (D-α-tocopherol polyethylene glycol succinate, TPGS, or vitamin E TPGS), methylcellulose, carboxymethylcellulose, sodium carboxymethylcellulose, methylacrylate, ethylacrylate, co-polymers of methyl and ethyl acrylate, hydroxypropylmethylcellulose acetate succinate, gelatin, maize starch, pea starch, modified maize starch, potato starch, modified potato starch, sodium starch glycolate, croscarmellose, crospovidone, copovidone, polyethylene glycol, polypropylene glycol, polyethylene and polypropylene glycol copolymers, polyvinylalcohol, polyvinylalcohol and polyethylene oxide copolymers. Copolymers of the foregoing polymers, where applicable, may also be used. Copolymers may be block, branched or terminal copolymers. In an embodiment, the polymer exhibits swelling, binding, or gelling properties that inhibit the disintegration, dissolution, and erosion of the pharmaceutical composition in order to prolong dissolution or to increase total dissolution. In an embodiment, the inclusion of the polymer increases dissolution rate and extent of dissolution over the use of an acidulant alone. The swelling, binding or gelling properties are pH-dependent in one embodiment, wherein the polymer swells, binds, or gels at one pH or range of pH in a different manner than at another pH. In one embodiment this may decrease dissolution at a lower pH than at a higher pH or vice versa. In another embodiment this leads to similar dissolution of voruciclib, including any voruciclib free base polymorph described herein, or any voruciclib salt polymorph described herein, in acidic, neutral or basic pH. This leads to similar plasma exposure independent of stomach pH.

The dissolution profile of a formulation containing one or more swelling, gelling, or binding excipients may exhibit a zero, first, or second differential rate order at one or more pH value or a mixture of different rate orders at different pH values. In an embodiment, a pharmaceutical composition will provide a constant level of drug into the gastrointestinal tract of a mammal by dissolution. Where voruciclib, including any voruciclib free base polymorph described herein, or any voruciclib salt polymorph described herein, is absorbed, this leads to a sustained plasma level of drug over a period, delays the tₘₐₓ, and reduces the cₘₐₓ of an equivalent dose of an immediate release formulation voruciclib, including any voruciclib free base polymorph described herein, or any voruciclib salt polymorph described herein. In another embodiment this leads to similar exposure in a mammal regardless of stomach pH.

### Voruciclib Solid Formulation

In one embodiment, the present disclosure provides a solid formulation comprising voruciclib malonate. In one embodiment, the solid formulation comprises between about 10% to 35% w/w, about 12% to 32% w/w; about 14% to 30% w/w; about 16% to 28% w/w, about 18% to 26% w/w; or about 20% to 24% w/w voruciclib malonate. In one embodiment, the formulation comprises about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, or about 35% w/w voruciclib malonate. In one embodiment, the formulation comprises about 20 mg to 100 mg, about 25 mg to 95 mg, about 30 mg to 90 mg, about 35 mg to 85 mg, about 40 mg to 80 mg, about 45 mg to 75 mg, about 50 mg to 70 mg, or about 55 mg to 65 mg voruciclib malonate. In one embodiment, the formulation comprises about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, or about 70 mg voruciclib malonate. In another embodiment, the formulation comprises between about 80 mg to 160 mg, about 85 mg to 155 mg, about 90 mg to 150 mg, about 95 mg to 145 mg, about 100 mg to 140 mg, about 105 mg to 135 mg, about 110 mg to 130 mg, about 115 mg to 125 mg, or about 120 mg to 124 mg voruciclib malonate. In one embodiment, the formulation comprises about 112 mg, about 113 mg, about 114 mg, about 115 mg, about 116 mg, about 117 mg, about 118 mg, about 119 mg, about 120 mg, about 121 mg, about 122 mg, about 123 mg, about 124 mg, about 125 mg, about 126 mg, about 127 mg, about 128 mg, about 129 mg, about 130 mg, about 131 mg, or about 132 mg voruciclib malonate.

In one embodiment, the solid formulation further comprises microcrystalline cellulose. In one embodiment, the formulation comprises between about 2% to 40% w/w, about 4% to 35% w/w; about 4% to 30% w/w; about 4% to 25% w/w; about 4% to 20% w/w; about 10% to 20% w/w; or about 12% to 16% w/w microcrystalline cellulose. In one embodiment, the formulation comprises about 5%, about 6%, about 7%, about 8%, about 9%, about 10%, about 11%, about 12% about 13%, about 14%, about 15%, about 16%, about 17%, about 18%, about 19%, about 20%, about 21%, about 22%, about 23%, or about 24% w/w microcrystalline cellulose. In one embodiment, the formulation comprises between about 1 mg to 80 mg, about 5 mg to 75 mg, about 10 mg to 70 mg, about 15 mg to 65 mg, about 20 mg to 60 mg, about 25 mg to 55 mg, about 30 mg to 50 mg, about 35 mg to 45 mg, or about 38 mg to 42 mg microcrystalline cellulose. In one embodiment, the formulation comprises about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, or about 50 mg microcrystalline cellulose. In another embodiment, the formulation comprises between about 20 mg to 120 mg, about 25 mg to 115 mg, about 30 mg to 110 mg, about 35 mg to 105 mg, about 40 mg to 100 mg, about 45 mg to 95 mg, about 50 mg to 90 mg, about 55 mg to 85 mg, about 60 mg to 85 mg, about 65 mg to 85 mg, about 70 mg to 85 mg, about 75 mg to 85 mg, or about 78 mg to 82 mg microcrystalline cellulose. In one embodiment, the formulation comprises about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, or about 90 mg microcrystalline cellulose.

In one embodiment, the solid formulation further comprises lactose monohydrate. In one embodiment, the formulation comprises between about 1% to 50% w/w, about 1% to 45% w/w; about 1% to 40% w/w; about 1% to 35% w/w; about 1% to 30% w/w, about 1% to 25% w/w; about 1% to 20% w/w; about 1% to 15% w/w, about 5% to 15% w/w, or about 8% to 12% w/w lactose monohydrate. In one embodiment, the formulation comprises about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% w/w lactose monohydrate. In one embodiment, the formulation comprises about 1 mg to 50 mg, about 1 mg to 45 mg, about 1 mg to 40 mg, about 5 mg to 40 mg, about 10 mg to 40 mg, about 10 mg to 35 mg, about 15 mg to 35 mg, about 20 mg to 35 mg, about 25 mg to 35 mg, or about 25 mg to 30 mg lactose monohydrate. In one embodiment, the formulation comprises about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, or about 40 mg lactose monohydrate. In another embodiment, the solid formulation comprises between about 5 mg to 90 mg, about 10 mg to 85 mg, about 15 mg to 80 mg, about 20 mg to 75 mg, about 25 mg to 70 mg, about 30 mg to 65 mg, about 35 mg to 60 mg, about 40 mg to 55 mg, or about 45 mg to 50 mg lactose monohydrate. In one embodiment, the formulation comprises about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, or about 55 mg lactose monohydrate.

In one embodiment, the solid formulation further comprises dibasic calcium phosphate dihydrate. In one embodiment, the formulation comprises between about 5% to 80% w/w; about 10% to 75% w/w; about 15% to 70% w/w; about 20% to 65% w/w; about 25% to 60% w/w; about 30% to 55% w/w; about 35% to 50% w/w; or about 40% to 45% w/w dibasic calcium phosphate dihydrate. In one embodiment, the formulation comprises about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, or about 52% w/w dibasic calcium phosphate dihydrate. In one embodiment, the formulation comprises between about 80 mg to 160 mg, about 85 mg to 155 mg, about 90 mg to 150 mg, about 95 mg to 145 mg, about 100 mg to 140 mg, about 105 mg to 135 mg, about 110 mg to 130 mg, about 115 mg to 125 mg, or about 120 mg to 124 mg dibasic calcium phosphate dihydrate. In one embodiment, the formulation comprises about 112 mg, about 113 mg, about 114 mg, about 115 mg, about 116 mg, about 117 mg, about 118 mg, about 119 mg, about 120 mg, about 121 mg, about 122 mg, about 123 mg, about 124 mg, about 125 mg, about 126 mg, about 127 mg, about 128 mg, about 129 mg, about 130 mg, about 131 mg, or about 132 mg dibasic calcium phosphate dihydrate. In another embodiment, the solid formulation comprises between about 200 mg to 300 mg, about 205 mg to 295 mg, about 210 mg to 290 mg, about 215 mg to 285 mg, about 220 mg to 280 mg, about 225 mg to 275 mg, about 230 mg to 270 mg, about 235 mg to 265 mg, about 235 mg to 260 mg, about 235 mg to 255 mg, about 235 mg to 250 mg, or about 240 mg to 245 mg dibasic calcium phosphate dihydrate. In one embodiment, the formulation comprises about 230 mg, about 231 mg, about 232 mg, about 233 mg, about 234 mg, about 235 mg, about 236 mg, about 237 mg, about 238 mg, about 239 mg, about 240 mg, about 241 mg, about 242 mg, about 243 mg, about 244 mg, about 245 mg, about 246 mg, about 247 mg, about 248 mg, about 249 mg, or about 250 mg dibasic calcium phosphate dihydrate.

In one embodiment, the solid formulation further comprises sodium bicarbonate. In one embodiment, the formulation comprises between about 0.01% to 20% w/w; about 0.01% to 18% w/w; about 0.01% to 16% w/w; about 0.01% to 14% w/w; about 0.01% to 12% w/w; about 0.01% to 10% w/w; about 1% to 10% w/w; about 1% to 8% w/w; about 1% to 6% w/w, or about 2% to 6% w/w sodium bicarbonate. In one embodiment, the formulation comprises about 0.5%, about 1%, about 2%, about 3%, about 4%, about 5%, about 6%, about 7%, about 8%, about 9%, about 10%, about 11%, about 13%, about 14, or about 15% w/w sodium bicarbonate. In one embodiment, the formulation comprises between about 1 mg and 40 mg, about 1 mg and 35 mg, about 1 mg and 30 mg, about 1 mg and 25 mg, about 1 mg and 20 mg, about 5 mg and 15 mg, or about 8 mg and 14 mg sodium bicarbonate. In one embodiment, the formulation comprises about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, or about 30 mg sodium bicarbonate. In another embodiment, the solid formulation comprises between about 5 mg and 80 mg, about 10 mg and 75 mg, about 10 mg and 70 mg, about 10 mg and 65 mg, about 10 mg to 60 mg, about 10 mg to 55 mg, about 10 mg to 50 mg, about 10 mg to 45 mg, about 10 mg to 40 mg, about 10 mg to 35 mg, about 10 mg to 30 mg, about 15 mg to 25 mg, or about 18 mg to 24 mg sodium bicarbonate. In one embodiment, the formulation comprises about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, or about 32 mg sodium bicarbonate.

In one embodiment, the solid formulation comprises between about 0.01% to 40% w/w; about 0.01% to 35% w/w; about 0.01% to 30% w/w; about 0.01% to 25%, about 0.01% to 20%, about 0.01% to 15% w/w; about 0.01% w/w to 10% w/w; about 4% to about 8% w/w sodium starch glycolate. In one embodiment, the formulation comprises about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% w/w sodium starch glycolate. In one embodiment, the formulation comprises between about 1 mg to 40 mg, about 1 mg to 35 mg, about 1 mg to 30 mg, about 1 mg to 25 mg, about 5 mg to 25 mg, about 10 mg to 25 mg, about 10 mg to 20 mg, or about 15 mg to 20 mg sodium starch glycolate. In one embodiment, the formulation comprises about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, or about 26 mg sodium starch glycolate. In another embodiment, the formulation comprises between about 5 mg to 70 mg, about 10 mg to 65 mg, about 15 mg to 60 mg, about 20 mg to 55 mg, about 25 mg to 50 mg, about 25 mg to 45 mg, about 25 mg to 40 mg, or about 30 mg to 35 mg sodium starch glycolate. In one embodiment, the formulation comprises about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, or about 43 mg sodium starch glycolate.

In one embodiment, the solid formulation further comprises magnesium stearate. In one embodiment, the formulation comprises between about 0.001% to 20% w/w; about 0.001% to 18% w/w; about 0.001% to 16% w/w; about 0.001% to 14% w/w, about 0.001% to 12% w/w; about 0.001% to 10% w/w, about 0.01% to 10% w/w; about 0.01% to 8% w/w, about 0.01% to 6%, about 0.01% to 4%, or about 1% to 4% w/w magnesium stearate. In one embodiment, the formulation comprises about 0.01%, about 0.05%, about 0.1%, about 0.5%, about 1%, about 2%, about 3%, about 4%, about 5%, about 6%, about 7%, about 8%, about 9%, about 10%, about 11% or about 12% w/w magnesium stearate. In one embodiment, the formulation comprises between about 0.001 mg to 15 mg, about 0.001 mg to 12 mg, about 0.001 mg to 10 mg, about 0.001 mg to 8 mg, about 0.001 mg to 6 mg, about 0.01 mg to 6 mg, about 0.1 mg to 6 mg, or about 2.5 mg to about 4.5 mg magnesium stearate. In one embodiment, the formulation comprises about 0.1 mg, about 0.5 mg, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg or about 14 mg magnesium stearate. In another embodiment, the formulation comprises between about 0.1 mg to 25 mg, about 0.1 mg to 22 mg, about 0.1 mg to 20 mg, about 0.1 mg to 18 mg, about 0.1 mg to 16 mg, about 0.1 mg to 14 mg, about 0.1 mg to 12 mg, about 0.1 mg to 10 mg, about 2 mg to 8 mg, or about 3 mg to 7 mg magnesium stearate. In one embodiment, the formulation comprises about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, or about 15 mg magnesium stearate.

In one embodiment, the solid formulation further comprises colloidal silicon dioxide. In one embodiment, the solid formulation comprises between about 0.001% to 20% w/w, about 0.001% to 18% w/w; about 0.001% to 16% w/w; about 0.001% to 14% w/w; about 0.001% to 12% w/w; about 0.001% to 10% w/w; about 0.01% to 10% w/w; about 0.01% to 8% w/w; about 0.01% to 6%, about 0.01% to 4%, or about 1% to 4% w/w colloidal silicon dioxide. In one embodiment, the formulation comprises about 0.01%, about 0.05%, about 0.1%, about 0.5%, about 1%, about 2%, about 3%, about 4%, about 5%, about 6%, about 7%, about 8%, about 9%, about 10%, about 11% or about 12% w/w colloidal silicon dioxide. In one embodiment, the formulation comprises between about 0.001 mg to 15 mg, about 0.001 mg to 12 mg, about 0.001 mg to 10 mg, about 0.001 mg to 8 mg, about 0.001 mg to 6 mg, about 0.01 mg to 6 mg, about 0.1 mg to 6 mg, or about 2.5 mg to about 4.5 mg colloidal silicon dioxide. In one embodiment, the formulation comprises about 0.1 mg, about 0.5 mg, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg or about 14 mg colloidal silicon dioxide. In another embodiment, the formulation comprises between about 0.1 mg to 30 mg, about 0.1 mg to 25 mg, about 0.1 mg to 20 mg, about 0.1 mg to 18 mg, about 0.1 mg to 15 mg, about 1 mg to 14 mg, about 1 mg to 12 mg, about 1 mg to 10 mg, about 1 mg to 8 mg, or about 3 mg to 8 mg colloidal silicon dioxide. In one embodiment, the formulation comprises about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, or about 15 mg colloidal silicon dioxide.

In one embodiment, the solid formulation is a tablet. In one embodiment, the tablet is coated with a film coating. In one embodiment, the film coating is Opadry II 85F18422 White.

In one embodiment, the solid formulation is a tablet comprising about 61 mg voruciclib malonate, which is about 50 mg free base voruciclib (i.e. a 50 mg tablet). In one embodiment, the 50 mg tablet further comprises about 40 mg microcrystalline cellulose, about 24 mg lactose monohydrate, about 121 mg dibasic calcium phosphate dihydrate, about 11 mg sodium bicarbonate, about 17 mg sodium starch glycolate, about 3 mg colloidal silicon dioxide, and about 3 mg magnesium stearate.

In another embodiment, the solid formulation is a tablet comprising about 122 mg voruciclib malonate, which is about 100 mg free base voruciclib (i.e., a 100 mg tablet). In one embodiment, the 100 mg tablet further comprises about 80 mg microcrystalline cellulose, about 48 mg lactose monohydrate, about 243 mg dibasic calcium phosphate dihydrate, about 22 mg sodium bicarbonate, about 34 mg sodium starch glycolate, about 6 mg colloidal silicon dioxide, and about 6 mg magnesium stearate.

Methods of Treating Solid Tumor Cancers, Hematological Malignancies. Inflammatory Diseases. Autoimmune Disorders. Immune Disorders, and Other Diseases

The pharmaceutical compositions described herein can be used in a method for treating diseases. In preferred embodiments, they are for use in treating hyperproliferative disorders. They may also be used in treating other disorders as described herein and in the following paragraphs.

In some embodiments, the disclosure provides a method of treating a hyperproliferative disorder in a mammal that comprises administering to the mammal a therapeutically effective amount of a crystalline solid form of voruciclib, including any voruciclib free base polymorph described herein, or any voruciclib salt polymorph described herein, or a pharmaceutical composition comprising a crystalline solid form of voruciclib, including any voruciclib free base polymorph described herein, or any voruciclib salt polymorph described herein, as described herein. In preferred embodiments, the mammal is a human. In some embodiments, the hyperproliferative disorder is cancer. In preferred embodiments, the cancer is selected from the group consisting of chronic lymphocytic leukemia, non-Hodgkin's lymphoma, diffuse large B-cell lymphoma, mantle cell lymphoma, follicular lymphoma, and Waldenström's macroglobulinemia. In preferred embodiments, the cancer is selected from the group consisting of non-Hodgkin's lymphomas (such as diffuse large B-cell lymphoma), acute myeloid leukemia, thymus, brain, lung, squamous cell, skin, eye, retinoblastoma, intraocular melanoma, oral cavity and oropharyngeal, bladder, gastric, stomach, pancreatic, bladder, breast, cervical, head, neck, renal, kidney, liver, ovarian, prostate, colorectal, bone (e.g., metastatic bone), esophageal, testicular, gynecological, thyroid, CNS, PNS, AIDS-related (e.g., lymphoma and Kaposi's sarcoma), viral-induced cancers such as cervical carcinoma (human papillomavirus), B-cell lymphoproliferative disease and nasopharyngeal carcinoma (Epstein-Barr virus), Kaposi's sarcoma and primary effusion lymphomas (Kaposi's sarcoma herpesvirus), hepatocellular carcinoma (hepatitis B and hepatitis C viruses), and T-cell leukemias (Human T-cell leukemia virus-1), B cell acute lymphoblastic leukemia, Burkitt's leukemia, juvenile myelomonocytic leukemia, hairy cell leukemia, Hodgkin's disease, multiple myeloma, mast cell leukemia, and mastocytosis. In selected embodiments, the method relates to the treatment of a non-cancerous hyperproliferative disorder such as benign hyperplasia of the skin (e.g., psoriasis), restenosis, or prostate conditions (e.g., benign prostatic hypertrophy (BPH)). In some embodiments, the hyperproliferative disorder is an inflammatory, immune, or autoimmune disorder. In some embodiments, the hyperproliferative disorder is selected from the group consisting of tumor angiogenesis, chronic inflammatory disease, rheumatoid arthritis, atherosclerosis, inflammatory bowel disease, skin diseases such as psoriasis, eczema, and scleroderma, diabetes, diabetic retinopathy, retinopathy of prematurity, age-related macular degeneration, hemangioma, glioma and melanoma, ulcerative colitis, atopic dermatitis, pouchitis, spondylarthrites, uveitis, Behcet's disease, polymyalgia rheumatica, giant-cell arteritis, sarcoidosis, Kawasaki disease, juvenile idiopathic arthritis, hidratenitis suppurativa, Sjögren's syndrome, psoriatic arthritis, juvenile rheumatoid arthritis, ankylosing spondylitis, Crohn's disease, lupus, and lupus nephritis. In an embodiment, the solid form of voruciclib, including any voruciclib free base polymorph described herein, or any voruciclib salt polymorph described herein, in any of the foregoing embodiments is selected from voruciclib malonate, voruciclib dibenzoyl-tartrate, voruciclib phosphate, voruciclib oxalate, and voruciclib napadisylate, each as described herein.

In an embodiment, the method of any of the foregoing embodiments further includes the step of administering an acid reducing agent to the mammal. In an embodiment, the acid reducing agent is selected from the group consisting of proton pump inhibitors, such as omeprazole, esomeprazole, lansoprazole, dexlansoprazole, pantoprazole, rabeprazole, and ilaprazole; H₂ receptor antagonists, such as cimetidine, ranitidine, and famotidine; and antacids such as bicarbonates, carbonates, and hydroxides of aluminum, calcium, magnesium, potassium, and sodium.

In some embodiments, the disclosure provides pharmaceutical compositions of a solid form of voruciclib, including any voruciclib free base polymorph described herein, or any voruciclib salt polymorph described herein, for use in the treatment of cancers such as thymus cancer, brain cancer (e.g., glioma), lung cancer, squamous cell cancer, skin cancer (e.g., melanoma), eye cancer, retinoblastoma cancer, intraocular melanoma cancer, oral cavity cancer, oropharyngeal cancer, bladder cancer, gastric cancer, stomach cancer, pancreatic cancer, bladder cancer, breast cancer, cervical cancer, head and neck cancer, renal cancer, kidney cancer, liver cancer, ovarian cancer, prostate cancer, colorectal cancer, colon cancer, esophageal cancer, testicular cancer, gynecological cancer, ovarian cancer, thyroid cancer, CNS cancer, PNS cancer, AIDS-related cancer (e.g., lymphoma and Kaposi's sarcoma), viral-induced cancer, and epidermoid cancer. In some embodiments, the disclosure provides pharmaceutical compositions of a solid form of voruciclib, including any voruciclib free base polymorph described herein, or any voruciclib salt polymorph described herein, for the treatment of a non-cancerous hyperproliferative disorder such as benign hyperplasia of the skin (e.g., psoriasis), restenosis, or prostate (e.g., benign prostatic hypertrophy (BPH)). In some embodiments, the disclosure provides pharmaceutical compositions of a solid form of voruciclib, including any voruciclib free base polymorph described herein, or any voruciclib salt polymorph described herein, for use in the treatment of disorders such as myeloproliferative disorders (MPDs), myeloproliferative neoplasms, polycythemia vera (PV), essential thrombocythemia (ET), primary myelofibrosis (PMF), myelodysplastic syndrome, chronic myelogenous leukemia (BCR-ABLI-positive), chronic neutrophilic leukemia, chronic eosinophilic leukemia, or mastocytosis. The disclosure also provides compositions for use in treating a disease related to vasculogenesis or angiogenesis in a mammal which can manifest as tumor angiogenesis, chronic inflammatory disease such as rheumatoid arthritis, inflammatory bowel disease, atherosclerosis, skin diseases such as psoriasis, eczema, and scleroderma, diabetes, diabetic retinopathy, retinopathy of prematurity, age-related macular degeneration, and hemangioma. In an embodiment, the solid form of voruciclib in any of the foregoing embodiments is selected from voruciclib malonate, voruciclib dibenzoyl-tartrate, voruciclib phosphate, voruciclib oxalate, and voruciclib napadisylate, each as described herein.

In some embodiments, the disclosure provides a method of treating a solid tumor cancer with a composition including a solid form of voruciclib, including any voruciclib free base polymorph described herein, or any voruciclib salt polymorph described herein. In some embodiments, the disclosure provides a method of treating pancreatic cancer, breast cancer, ovarian cancer, melanoma, lung cancer, squamous cell carcinoma including head and neck cancer, or a blood cancer. In an embodiment, the disclosure provides a method for treating pancreatic cancer, breast cancer, ovarian cancer, melanoma, lung cancer, head and neck cancer, colorectal cancer, or a blood cancer using a combination of a solid form of voruciclib, including any voruciclib free base polymorph described herein, or any voruciclib salt polymorph described herein, and a second agent selected from the group consisting of bendamustine, venetoclax, vemurafenib, abraxane, enasidenib, pomalidomide, lenalidomide, azacitidine, decitabine, a hypomethylating agent, gemcitabine, albumin-bound paclitaxel, rituximab, obinutuzumab, ofatumumab, pembrolizumab, nivolumab, durvalumab, avelumab, atezolizumab, bortezomib, marizomib, ixazomib, disulfiram, epigallocatechin-3-gallate, salinosporamide A, carfilzomib, ONX 0912, CEP-18770, MLN9708, epoxomicin, or MG13. In an embodiment, the disclosure provides a method for treating pancreatic cancer, breast cancer, ovarian cancer, melanoma, lung cancer, head and neck cancer, colorectal cancer, or a blood cancer using a combination of a CDK inhibitor and bendamustine, venetoclax, vemurafenib, abraxane, enasidenib, pomalidomide, lenalidomide, azacitidine, decitabine, a hypomethylating agent, gemcitabine, albumin-bound paclitaxel, rituximab, obinutuzumab, ofatumumab, pembrolizumab, nivolumab, durvalumab, avelumab, atezolizumab. For certain methods described herein, the proteasome inhibitor is selected from bortezomib, marizomib, ixazomib, disulfiram, epigallocatechin-3-gallate, salinosporamide A, carfilzomib, ONX 0912, CEP-18770, MLN9708, epoxomicin, or MG13, wherein the CDK inhibitor is a solid form of voruciclib, including any voruciclib free base polymorph described herein, or any voruciclib salt polymorph described herein. In an embodiment, the solid form of voruciclib in any of the foregoing embodiments is selected from voruciclib malonate, voruciclib dibenzoyl-tartrate, voruciclib phosphate, voruciclib oxalate, and voruciclib napadisylate, each as described herein.

In some embodiments, the disclosure provides a method of treating a solid tumor cancer with a composition including a solid form of voruciclib, including any voruciclib free base polymorph described herein, or any voruciclib salt polymorph described herein. In some embodiments, the disclosure provides a method of treating pancreatic cancer, breast cancer, ovarian cancer, melanoma, lung cancer, squamous cell carcinoma including head and neck cancer. In an embodiment, the disclosure provides a method for treating pancreatic cancer, breast cancer, ovarian cancer, melanoma, lung cancer, head and neck cancer, and colorectal cancer using a solid form of voruciclib, including any voruciclib free base polymorph described herein, or any voruciclib salt polymorph described herein. In an embodiment, the solid form of voruciclib in any of the foregoing embodiments is selected from voruciclib malonate, voruciclib dibenzoyl-tartrate, voruciclib phosphate, voruciclib oxalate, and voruciclib napadisylate, each as described herein.

In some embodiments, the disclosure relates to a method of treating an inflammatory, immune, or autoimmune disorder in a mammal with a composition including a solid form of voruciclib, including any voruciclib free base polymorph described herein, or any voruciclib salt polymorph described herein. In selected embodiments, the disclosure also relates to a method of treating a disease with a composition including a solid form of voruciclib, including any voruciclib free base polymorph described herein, or any voruciclib salt polymorph described herein, wherein the disease is selected from the group consisting of tumor angiogenesis, chronic inflammatory disease, rheumatoid arthritis, atherosclerosis, inflammatory bowel disease, skin diseases such as psoriasis, eczema, and scleroderma, diabetes, diabetic retinopathy, retinopathy of prematurity, age-related macular degeneration, hemangioma, glioma and melanoma, ulcerative colitis, atopic dermatitis, pouchitis, spondylarthrites, uveitis, Behcet's disease, polymyalgia rheumatica, giant-cell arteritis, sarcoidosis, Kawasaki disease, juvenile idiopathic arthritis, hidratenitis suppurativa, Sjögren's syndrome, psoriatic arthritis, juvenile rheumatoid arthritis, ankylosing spondylitis, Crohn's Disease, lupus, and lupus nephritis. In an embodiment, the solid form of voruciclib in any of the foregoing embodiments is selected from voruciclib malonate, voruciclib dibenzoyl-tartrate, voruciclib phosphate, voruciclib oxalate, and voruciclib napadisylate, each as described herein.

In some embodiments, the disclosure relates to a method of treating a hyperproliferative disorder in a mammal with a composition including a solid form of voruciclib, including any voruciclib free base polymorph described herein, or any voruciclib salt polymorph described herein, wherein the hyperproliferative disorder is a B cell hematological malignancy selected from the group consisting of chronic lymphocytic leukemia (CLL), small lymphocytic leukemia (SLL), non-Hodgkin's lymphoma (NHL), diffuse large B cell lymphoma (DLBCL), follicular lymphoma (FL), mantle cell lymphoma (MCL). Hodgkin's lymphoma. B cell acute lymphoblastic leukemia (B-ALL), Burkitt's lymphoma. Waldenström's macroglobulinemia (WM), Burkitt's lymphoma, multiple myeloma, myelodysplastic syndromes, or myelofibrosis. In some embodiments, the disclosure relates to a method of treating a hyperproliferative disorder in a mammal with a composition including a solid form of voruciclib, including any voruciclib free base polymorph described herein, or any voruciclib salt polymorph described herein, wherein the hyperproliferative disorder is selected from the group consisting of chronic myelocytic leukemia, acute myeloid leukemia, DLBCL (including activated B-cell (ABC) and germinal center B-cell (GCB) subtypes), follicle center lymphoma. Hodgkin's disease, multiple myeloma, indolent non-Hodgkin's lymphoma, and mature B-cell ALL. In an embodiment, the solid form of voruciclib in any of the foregoing embodiments is selected from voruciclib malonate, voruciclib dibenzoyl-tartrate, voruciclib phosphate, voruciclib oxalate, and voruciclib napadisylate, each as described herein.

In some embodiments, the hyperproliferative disorder is a subtype of CLL. A number of subtypes of CLL have been characterized. CLL is often classified for immunoglobulin heavy-chain variable-region (IgV_{H}) mutational status in leukemic cells. R. N. Damle, et al., Blood 1999, 94. 1840-47; T. J. Hamblin, et al., Blood 1999, 94. 1848-54. Patients with IgV_{H} mutations generally survive longer than patients without IgV_{H} mutations. ZAP70 expression (positive or negative) is also used to characterize CLL. L. Z. Rassenti, et al., N. Engl. J. Med. 2004, 351. 893-901. The methylation of ZAP-70 at CpG3 is also used to characterize CLL, for example by pyrosequencing. R. Claus, et al., J. Clin. Oncol. 2012, 30. 2483-91; J. A. Woyach, et al., Blood 2014, 123. 1810-17. CLL is also classified by stage of disease under the Binet or Rai criteria. J. L. Binet, et al., Cancer 1977, 40. 855-64; K. R. Rai, T. Han, Hematol. Oncol. Clin. North Am. 1990, 4. 447-56. Other common mutations, such as 11q deletion, 13q deletion, and 17p deletion can be assessed using well-known techniques such as fluorescence in situ hybridization (FISH). In an embodiment, the disclosure relates to a method of treating a CLL in a human, wherein the CLL is selected from the group consisting of IgV_{H} mutation negative CLL, ZAP-70 positive CLL, ZAP-70 methylated at CpG3 CLL, CD38 positive CLL, chronic lymphocytic leukemia characterized by a 17p13.1 (17p) deletion, and CLL characterized by a 11q22.3 (11q) deletion.

In some embodiments, the hyperproliferative disorder is a CLL wherein the CLL has undergone a Richter's transformation. Methods of assessing Richter's transformation, which is also known as Richter's syndrome, are described in Jain and O'Brien, Oncology, 2012, 26. 1146-52. Richter's transformation is a subtype of CLL that is observed in 5-10% of patients. It involves the development of aggressive lymphoma from CLL and has a generally poor prognosis.

In some embodiments, the hyperproliferative disorder is a CLL or SLL in a patient, wherein the patient is sensitive to lymphocytosis. In an embodiment, the disclosure relates to a method of treating CLL or SLL in a patient, wherein the patient exhibits lymphocytosis caused by a disorder selected from the group consisting of a viral infection, a bacterial infection, a protozoal infection, or a post-splenectomy state. In an embodiment, the viral infection in any of the foregoing embodiments is selected from the group consisting of infectious mononucleosis, hepatitis, and cytomegalovirus. In an embodiment, the bacterial infection in any of the foregoing embodiments is selected from the group consisting of pertussis, tuberculosis, and brucellosis.

In some embodiments, the hyperproliferative disorder is a blood cancer. In certain embodiments, the blood cancer is leukemia, such as acute myeloid leukemia (AML), chronic myeloid leukemia (CML), acute lymphocytic lymphoma (ALL), and chronic lymphocytic leukemia (CLL). In certain embodiments, the blood cancer is a non-Hodgkin lymphoma, such as B-cell or T-cell lymphoma. B-cell lymphomas include diffuse large B-cell lymphoma (DLBCL), primary mediastinal B-cell lymphoma, intravascular large B-cell lymphoma, follicular lymphoma, small lymphocytic lymphoma (SLL), mantle cell lymphoma, marginal zone B-cell lymphomas, extranodal marginal zone B-cell lymphomas, nodal marginal zone B-cell lymphoma, splenic marginal zone B-cell lymphoma. Burkitt lymphoma, lymphoplasmacytic lymphoma, and primary central nervous system lymphoma. T-cell lymphomas include precursor T-lymphoblastic lymphoma, peripheral T-cell lymphomas, cutaneous T-cell lymphomas, adult T-cell lymphoma with subtypes: smoldering chronic, acute, and lymphoma, angioimmunoblastic T-cell lymphoma, extranodal natural killer/T-cell lymphoma, nasal type, enteropathy-associated intestinal T-cell lymphoma (EATL) with subtypes I and II, and anaplastic large cell lymphoma (ALCL). Methods of using voruciclib for treating blood cancers are described in WO 2017/172826, incorporated by reference herein in its entirety.

The following clauses describe certain embodiments.

Clause 1. A method of treating a disease or disorder in a subject, the method comprising administering to the subject a therapeutically effective amount of voruciclib, wherein the disease or disorder is selected from chronic lymphocytic leukemia, non-Hodgkin's lymphoma, diffuse large B-cell lymphoma, mantle cell lymphoma, follicular lymphoma. B-cell lymphoproliferative disease, B cell acute lymphoblastic leukemia, Waldenström's macroglobulinemia, Burkitt's leukemia, Hodgkin's disease, multiple myeloma, acute myeloid leukemia, juvenile myelomonocytic leukemia, hairy cell leukemia, mast cell leukemia, mastocytosis, myeloproliferative disorders (MPDs), myeloproliferative neoplasms, polycythemia vera (PV), essential thrombocythemia (ET), primary myelofibrosis (PMF), myelodysplastic syndrome, chronic myelogenous leukemia (BCR-ABLI-positive), chronic neutrophilic leukemia, chronic eosinophilic leukemia, primary central nervous system (CNS) lymphoma, primary multifocal lymphoma of peripheral nervous system (PNS), thymus cancer, brain cancer, glioblastoma, lung cancer, squamous cell cancer, skin cancer (e.g., melanoma), eye cancer, retinoblastoma, intraocular melanoma, oral cavity and oropharyngeal cancers, bladder cancer, gastric cancer, stomach cancer, pancreatic cancer, breast cancer, cervical cancer, head and neck cancer, renal cancer, kidney cancer, liver cancer, ovarian cancer, prostate cancer, colorectal cancer, bone cancer (e.g., metastatic bone cancer), esophageal cancer, testicular cancer, gynecological cancer, thyroid cancer, epidermoid cancer, AIDS-related cancer (e.g., lymphoma), viral-induced cervical carcinoma (human papillomavirus), nasopharyngeal carcinoma (Epstein-Barr virus), Kaposi's sarcoma, primary effusion lymphoma (Kaposi's sarcoma herpesvirus), hepatocellular carcinoma (hepatitis B and hepatitis C viruses), T-cell leukemias (Human T-cell leukemia virus-1), benign hyperplasia of the skin, restenosis, benign prostatic hypertrophy, tumor angiogenesis, chronic inflammatory disease, rheumatoid arthritis, atherosclerosis, inflammatory bowel disease, skin diseases such as psoriasis, eczema, and scleroderma, diabetes, diabetic retinopathy, retinopathy of prematurity, age-related macular degeneration, hemangioma, ulcerative colitis, atopic dermatitis, pouchitis, spondylarthrites, uveitis, Behcet's disease, polymyalgia rheumatica, giant-cell arteritis, sarcoidosis, Kawasaki disease, juvenile idiopathic arthritis, hidratenitis suppurativa, Sjögren's syndrome, psoriatic arthritis, juvenile rheumatoid arthritis, ankylosing spondylitis. Crohn's disease, lupus, and lupus nephritis.

Clause 2. A method of treating a hyperproliferative disease or disorder in a subject, the method comprising administering to the subject a therapeutically effective amount of voruciclib, wherein the hyperproliferative disease or disorder is selected from acute lymphoblastic leukemia, acute myeloid leukemia, chronic lymphocytic leukemia, non-Hodgkin's lymphoma, diffuse large B-cell lymphoma, mantle cell lymphoma, follicular lymphoma. B-cell lymphoproliferative disease, B cell acute lymphoblastic leukemia, and Waldenström's macroglobulinemia.

Clause 3. A method of treating a blood cancer in a subject, the method comprising administering to the subject a therapeutically effective amount of voruciclib.

Clause 4. The method of clause 3, wherein the blood cancer is selected from acute myeloid leukemia (AML), chronic myeloid leukemia (CML), acute lymphocytic lymphoma (ALL), and chronic lymphocytic leukemia (CLL).

Clause 5. A method of treating a hyperproliferative disease or disorder in a subject, the method comprising administering to the subject a therapeutically effective amount of voruciclib, wherein the hyperproliferative disease or disorder is a KRAS mutant cancer.

Clause 6. The method of clause 5, wherein the KRAS mutant cancer is characterized by a mutation selected from G12A, G12C, G12D, G12S. G12V. G13C, G13D, and Q61H.

Clause 7. The method of clause 5 or 6, wherein the cancer is selected from acute myeloid leukemia (AML), chronic myeloid leukemia (CML), acute lymphocytic lymphoma (ALL), and chronic lymphocytic leukemia (CLL), diffuse large B-cell lymphoma (DLBCL), primary mediastinal B-cell lymphoma, intravascular large B-cell lymphoma, follicular lymphoma, small lymphocytic lymphoma (SLL), mantle cell lymphoma, marginal zone B-cell lymphomas, extranodal marginal zone B-cell lymphomas, nodal marginal zone B-cell lymphoma, splenic marginal zone B-cell lymphoma, Burkitt lymphoma, lymphoplasmacytic lymphoma, and primary central nervous system lymphoma.

Clause 8. The method of any one of clauses 5 to 7, wherein the cancer is selected from pancreatic cancer, lung cancer, colorectal cancer, esophageal cancer, and ovarian cancer.

Clause 9. The method of any one of clauses 5 to 7, wherein the cancer is selected from NSCLC, SCLC, CRC, pancreatic, TNBC, melanoma, breast cancer, and liver cancer.

Clause 10. The method of any one of clauses I to 9, wherein the disease or disorder is a relapsed/refractory (R/R) disease or disorder.

Clause 11. The method of any one of clauses 1 to 10, wherein voruciclib comprises a voruciclib salt comprising a counterion corresponding to an acid selected from 1,5-naphthalenedisulfonic acid, 1-hydroxy-2-naphthoic acid, benzenesulfonic acid, benzoic acid, dibenzoyl-L-tartaric acid, ethanesulfonic acid, gentisic acid, hydrobromic acid, hydrochloric acid, maleic acid, malonic acid, oxalic acid, ortho-phosphoric acid, sulfuric acid, and p-toluenesulfonic acid.

Clause 12. The method of any one of clauses 1 to 10, wherein voruciclib comprises a crystal form of voruciclib, comprising voruciclib free base or a voruciclib salt comprising a counterion corresponding to an acid selected from 1,5-naphthalenedisulfonic acid, 1-hydroxy-2-naphthoic acid, benzenesulfonic acid, benzoic acid, dibenzoyl-L-tartaric acid, ethanesulfonic acid, gentisic acid, hydrobromic acid, hydrochloric acid, maleic acid, malonic acid, oxalic acid, ortho-phosphoric acid, sulfuric acid, and p-toluenesulfonic acid.

Clause 13. The method of any one of clauses 1 to 10, wherein voruciclib comprises a crystal form of voruciclib characterized by an X-ray powder diffraction pattern comprising one or more peaks selected from 7.30°±0.2°, 13.58°±0.2°, 14.06°±0.2°, 15.18°±0.2°, 15.66°±0.2°, 17.50°±0.2°, 18.94°±0.2°, 19.54°±0.2°, 22.22°±0.2°, 23.38°±0.2°, 24.10°±0.2°, 24.98°±0.2°, 25.94°±0.2°, 27.26°±0.2°, 28.50°±0.2°, and 32.82°±0.2°20.

Clause 14. The method of clause 13, wherein the crystal form comprises voruciclib malonate.

Clause 15. The method of any one of clauses 1 to 10, wherein voruciclib comprises a crystal form of voruciclib characterized by an X-ray powder diffraction pattern comprising one or more peaks selected from 5.06°±0.2°, 6.42°±0.2°, 9.34°±0.2°, 10.14°±0.2°, 12.30°±0.2°, 13.66°±0.2°, 14.14°±0.2°, 15.82°±0.2°, 17.02°±0.2°, 19.74°±0.2°, 20.38°±0.2°, 21.82°±0.2°, 22.66°±0.2°, 24.62°±0.2°, 25.78°±0.2°, 26.58°±0.2°, 28.66°±0.2°, and 29.98°±0.2° 2θ.

Clause 16. The method of clause 15, wherein the crystal form comprises voruciclib dibenzoyl-tartrate.

Clause 17. The method of any one of clauses 1 to 10, wherein voruciclib comprises a crystal form of voruciclib characterized by an X-ray powder diffraction pattern comprising one or more peaks selected from 4.94°±0.2°, 6.78°±0.2°, 9.34°±0.2°, 10.94°±0.2°, 12.70°±0.2°, 13.38°±0.2°, 14.90°±0.2°, 15.66°±0.2°, 17.54°±0.2°, 18.82°±0.2°, 22.02°±0.2°, 23.98°±0.2°, 24.78°±0.2°, 25.30°±0.2°, 26.66°±0.2°, and 29.98°±0.2°20.

Clause 18. The method of clause 17, wherein the crystal form comprises voruciclib phosphate.

Clause 19. The method of any one of clauses 1 to 10, wherein voruciclib comprises a crystal form of voruciclib characterized by an X-ray powder diffraction pattern comprising one or more peaks selected from 6.86°±0.2°, 12.66°±0.2°, 13.58°±0.2°, 14.74°±0.2°, 15.98°±0.2°, 19.38°±0.2°, 23.94°±0.2°, 24.78°±0.2°, and 25.94°±0.2° 2θ.

Clause 20. The method of clause 19, wherein the crystal form comprises voruciclib oxalate.

Clause 21. The method of any one of clauses 1 to 10, wherein voruciclib comprises a crystal form of voruciclib characterized by an X-ray powder diffraction pattern comprising one or more peaks selected from 9.02°±0.2°, 10.50°±0.2°, 11.06°±0.2°, 12.30°±0.2°, 12.82°±0.2°, 13.90°±0.2°, 14.82°±0.2°, 15.30°±0.2°, 15.94°±0.2°, 17.26°±0.2°, 19.34°±0.2°, 20.62°±0.2°, 22.18°±0.2°, 22.86°±0.2°, 24.58°±0.2°, 25.42°±0.2°, 25.86°±0.2°, 27.38°±0.2°, and 28.66°±0.2° 2θ.

Clause 22. The method of clause 21, wherein the crystal form comprises voruciclib napadisylate.

Clause 23. The method of any one of clauses 12 to 22, wherein the crystal form is a crystalline anhydrate.

Clause 24. The method of any one of clauses 12 to 22, wherein the crystal form is a crystalline hydrate.

Clause 25. The method of any one of clauses 1 to 10, wherein voruciclib comprises a crystal form of voruciclib malonate characterized by an X-ray powder diffraction pattern comprising one or more peaks selected from 6.36°±0.2° 2θ, 13.88°±0.2° 2θ, 7.31°±0.2° 2θ, 9.34°±0.2° 2θ, 10.05°±0.2° 2θ, 13.59°±0.2° 2θ, 14.08°±0.2° 2θ, 15.21°±0.2° 20, 15.67°±0.2° 2θ, 17.53°±0.2° 2θ, 18.70°±0.2° 2θ, 18.98°±0.2° 2θ, 19.38°±0.2° 2θ, 19.67°±0.2° 2θ, 20.16°±0.2° 2θ, 20.39°±0.2° 2θ, 21.01°±0.2° 2θ, 22.27°±0.2° 2θ, 23.35°±0.2° 2θ, 24.15°±0.2° 2θ, 24.67°±0.2° 2θ, 25.00°±0.2° 2θ, 25.18°±0.2° 2θ, 25.57°±0.2° 2θ, 25.93°±0.2° 2θ, 26.21°±0.2° 2θ, 27.19°±0.2° 2θ, and 27.38°±0.2° 2θ.

Clause 26. The method of any one of clauses 1 to 10, wherein voruciclib comprises a crystal form of voruciclib oxalate characterized by an X-ray powder diffraction pattern comprising one or more peaks selected from 6.86°±0.2° 2θ, 9.70°±0.2° 2θ, 10.84°±0.2° 20, 12.50°±0.2° 2θ, 12.66°±0.2° 2θ, 12.81°±0.2° 2θ, 13.41°±0.2° 2θ, 13.71°±0.2° 2θ, 14.54°±0.2° 2θ, 15.35°±0.2° 2θ, 15.83°±0.2° 2θ, 18.70°±0.2° 2θ, 19.00°±0.2° 2θ, 19.43°±0.2° 2θ, 19.62°±0.2° 2θ, 21.75°±0.2° 2θ, 22.75°±0.2° 2θ, 23.35°±0.2° 2θ, 23.47°±0.2° 2θ, 23.81°±0.2° 2θ, 23.98°±0.2° 2θ, 24.36°±0.2° 2θ, 24.60°±0.2° 2θ, 24.86°±0.2° 2θ, 25.11°±0.2° 2θ, 25.60°±0.2° 2θ, 25.75°±0.2° 2θ, and 26.25°±0.2° 2θ.

Clause 27. The method of any one of clauses 1 to 10, wherein voruciclib comprises a crystal form of voruciclib phosphate characterized by an X-ray powder diffraction pattern comprising one or more peaks selected from 4.93°±0.2° 2θ, 6.79°±0.2° 2θ, 9.35°±0.2° 2θ, 10.58°±0.2° 2θ, 10.91°±0.2° 2θ, 12.64°±0.2° 2θ, 13.35°±0.2° 2θ, 13.58°±0.2° 2θ, 14.81°±0.2° 2θ, 15.60°±0.2° 2θ, 17.18°±0.2° 2θ, 17.52°±0.2° 2θ, 18.32°±0.2° 2θ, 18.78°±0.2° 2θ, 19.34°±0.2° 2θ, 19.64°±0.2° 2θ, 19.78°±0.2° 2θ, 22.02°±0.2° 2θ, 23.20°±0.2° 2θ, 23.67°±0.2° 2θ, 24.00°±0.2° 2θ, 24.71°±0.2° 2θ, 25.21°±0.2° 2θ, 25.39°±0.2° 2θ, 26.55°±0.2° 2θ, 27.22°±0.2° 2θ, 28.07°±0.2° 2θ, and 29.90°±0.2° 2θ.

Clause 28. The method of any one of clauses 1 to 27, wherein voruciclib is administered at a daily dose between about 50 mg and about 100 mg, between about 100 mg and about 150 mg, between about 150 mg and about 200 mg, between about 200 mg and about 250 mg, between about 250 mg and about 300 mg, between about 300 mg and about 350 mg, between about 350 mg and about 400 mg, between about 400 mg and about 450 mg, between about 450 mg and about 500 mg, between about 500 mg and about 550 mg, between about 550 mg and about 600 mg, between about 600 mg and about 650 mg, between about 650 mg and about 700 mg, between about 700 mg and about 750 mg, between about 750 mg and about 800 mg, between about 800 mg and about 850 mg, between about 850 mg and about 900 mg, between about 900 mg and about 950 mg, or between about 950 mg and about 1,000 mg.

Clause 29. The method of any one of clauses 1 to 27, wherein voruciclib is administered at a daily dose of about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1,000 mg.

Clause 30. The method of any one of clauses 1 to 29, wherein voruciclib is administered daily for about one day, about two days, about three days, about 4 days, about 5 days, about 6 days, about 7 days, about 8 days, about 9 days, about 10 days, about 11 days, about 12 days, about 13 days, or about 14 days.

Clause 31. The method of any one of clauses 1 to 29, wherein voruciclib is administered daily for about one week, about two weeks, about three weeks, or about 4 weeks.

Clause 32. The method of any one of clauses 1 to 31, wherein voruciclib administration is paused for about one day, about two days, about three days, about 4 days, about 5 days, about 6 days, about 7 days, about 8 days, about 9 days, about 10 days, about 11 days, about 12 days, about 13 days, or about 14 days.

Clause 33. The method of any one of clauses 1 to 31, wherein voruciclib administration is paused for about one week, about two weeks, about three weeks, or about 4 weeks.

Clause 34. The method of any one of clauses 1 to 29, wherein voruciclib is administered on a 14 days on/14 days off schedule.

Clause 35. The method of any one of clauses 1 to 34, wherein voruciclib is administered for about one month, about two months, about three months, about 4 months, about 5 months, about 6 months, about 7 months, about 8 months, about 9 months, about 10 months, about 11 months, or about 12 months.

Clause 36. The method of any one of clauses 1 to 35, wherein voruciclib is administered in combination with an additional therapeutic agent.

Clause 37. The method of clause 36, wherein the therapeutic agent is an anticancer agent.

Clause 38. The method of clause 37, wherein the anticancer agent is selected from AMG510, MRTX849, Onvansertib, Volasertib, and ME-344.

Clause 39. The method of clause 38, wherein the anticancer agent is selected from a KRAS inhibitor, a TKI+RAF inhibitor, a RAF inhibitor, a RAF+MEK inhibitor, a MEK inhibitor, and an ERK inhibitor.

Clause 40. The method of clause 38, wherein the anticancer agent is a BCL-2 inhibitor selected from navitoclax, venetoclax, A-1155463, A-1331852, ABT-737, obatoclax, TW-37, A-1210477, AT101, HA14-1, BAM7, S44563, sabutoclax, UMI-77, gambogic acid, maritoclax, MIMI, methylprednisolone, iMAC2, Bax inhibitor peptide V5, Bax inhibitor peptide P5, Bax channel blocker, and ARRY 520 trifluoroacetate.

Clause 41. The method of clause 38, wherein the anticancer agent is a proteasome inhibitor selected from bortezomib, marizomib, ixazomib, disulfiram, epigallocatechin-3-gallate, salinosporamide A, carfilzomib, ONX 0912, CEP-18770, MLN9708, epoxomicin, MG132 and a pharmaceutically acceptable salt of any one thereof.

Clause 42. The method of any one of clauses 36 to 41, wherein the additional therapeutic agent is administered daily for about one day, about two days, about three days, about 4 days, about 5 days, about 6 days, about 7 days, about 8 days, about 9 days, about 10 days, about 11 days, about 12 days, about 13 days, or about 14 days.

Clause 43. The method of any one of clauses 36 to 41, wherein the additional therapeutic agent is administered daily for about one week, about two weeks, about three weeks, or about 4 weeks.

Clause 44. The method of any one of clauses 36 to 43, wherein the additional therapeutic agent administration is paused for about one day, about two days, about three days, about 4 days, about 5 days, about 6 days, about 7 days, about 8 days, about 9 days, about 10 days, about 11 days, about 12 days, about 13 days, or about 14 days.

Clause 45. The method of any one of clauses 36 to 43, wherein the additional therapeutic agent administration is paused for about one week, about two weeks, about three weeks, or about 4 weeks.

Clause 46. The method of any one of clauses 36 to 41, wherein the additional therapeutic agent is administered on a 14 days on/14 days off schedule.

Clause 47. The method of any one of clauses 36 to 46, wherein the additional therapeutic agent is administered for about one month, about two months, about three months, about 4 months, about 5 months, about 6 months, about 7 months, about 8 months, about 9 months, about 10 months, about 11 months, or about 12 months.

Clause 48. A voruciclib malonate formulation described herein.

Clause 101. A formulation comprising between about 15% to about 35% w/w voruciclib malonate and one or more pharmaceutically acceptable excipients.

Clause 102. The formulation of clause 101, comprising between about 18% to about 30% w/w voruciclib malonate.

Clause 103. The formulation of clause 101, comprising about 18%, about 19%, about 20%, about 21%, about 22%, about 23%, about 24%, about 25%, about 26%, about 27%, or about 28% w/w voruciclib malonate.

Clause 104. The formulation of clause 101, comprising between about 20% to about 23% w/w voruciclib malonate.

Clause 105. The formulation of any one of clauses 101 to 104, wherein the one or more pharmaceutically acceptable excipients comprise about 5% to about 37% w/w microcrystalline cellulose.

Clause 106. The formulation of any one of clauses 101 to 104, wherein the one or more pharmaceutically acceptable excipients comprise about 10%, about 11%, about 12%, about 13%, about 14%, about 15%, about 16%, about 17%, about 18%, about 19%, or about 20% w/w microcrystalline cellulose.

Clause 107. The formulation of any one of clauses 101 to 106, wherein the one or more pharmaceutically acceptable excipients comprise about 1% to about 48% w/w lactose monohydrate.

Clause 108. The formulation of any one of clauses 101 to 106, wherein the one or more pharmaceutically acceptable excipients comprise about 2%, about 3%, about 4%, about 5%, about 6%, about 7%, about 8%, about 9%, about 10%, about 11%, about 12%, about 13%, about 14%, or about 15% w/w lactose monohydrate.

Clause 109. The formulation of any one of clauses 101 to 108, wherein the one or more pharmaceutically acceptable excipients comprise about 20% to about 70% w/w dibasic calcium phosphate dihydrate.

Clause 110. The formulation of any one of clauses 101 to 108, wherein the one or more pharmaceutically acceptable excipients comprise about 34%, about 35%, about 36%, about 37%, about 38%, about 39%, about 40%, about 41%, about 42%, about 43%, about 44%, about 45%, about 46%, about 47%, about 48%, about 49%, or about 50% w/w dibasic calcium phosphate dihydrate.

Clause 111. The formulation of any one of clauses 101 to 110, wherein the one or more pharmaceutically acceptable excipients comprise about 0.1% to about 15% w/w sodium bicarbonate.

Clause 112. The formulation of any one of clauses 101 to 110, wherein the one or more pharmaceutically acceptable excipients comprise about 2%, about 3%, about 4%, about 5%, about 6%, about 7%, about 8%, about 9%, or about 10% w/w sodium bicarbonate.

Clause 113. The formulation of any one of claims 1 to 12, wherein the one or more pharmaceutically acceptable excipients comprise about 1% to about 20% w/w sodium starch glycolate.

Clause 114. The formulation of any one of clauses 101 to 112, wherein the one or more pharmaceutically acceptable excipients comprise about 2%, about 3%, about 4%, about 5%, about 6%, about 7%, about 8%, about 9%, about 10%, about 11%, or about 12% w/w sodium starch glycolate.

Clause 115. The formulation of any one of clauses 101 to 114, wherein the one or more pharmaceutically acceptable excipients comprise about 0.01% to about 10% w/w magnesium stearate.

Clause 116. The formulation of any one of clauses 101 to 114, wherein the one or more pharmaceutically acceptable excipients comprise about 0.1%, about 0.25%, about 0.5%, about 0.75%, about 1%, about 1.25%, about 1.5%, about 1.75%, about 2%, about 3%, about 4%, or about 5% w/w magnesium stearate.

Clause 117. The formulation of any one of clauses 101 to 116, wherein the one or more pharmaceutically acceptable excipients comprise about 0.01% to about 10% w/w colloidal silicon dioxide.

Clause 118. The formulation of any one of clauses 101 to 116, wherein the one or more pharmaceutically acceptable excipients comprise about 0.1%, about 0.25%, about 0.5%, about 0.75%, about 1%, about 1.25%, about 1.5%, about 1.75%, about 2%, about 3%, about 4%, or about 5% w/w colloidal silicon dioxide.

Clause 119. The formulation of any one of clauses 101 to 118, wherein the formulation is comprised into a tablet.

Clause 120. The formulation of clause 119, wherein the tablet is coated with a film coating.

Clause 121. The formulation of any one of clauses 101 to 120, wherein voruciclib malonate comprises a crystal form of voruciclib malonate characterized by an X-ray powder diffraction pattern comprising one or more peaks selected from 7.30°±0.2°, 13.58°±0.2°, 14.06°±0.2°, 15.18°±0.2°, 15.66°±0.2°, 17.50°±0.2°, 18.94°±0.2°, 19.54°±0.2°, 22.22°±0.2°, 23.38°±0.2°, 24.10°±0.2°, 24.98°±0.2°, 25.94°±0.2°, 27.26°±0.2°, 28.50°±0.2°, and 32.82°±0.2° 2θ.

Clause 122. The formulation any one of clauses 101 to 120, wherein voruciclib malonate comprises a crystal form of voruciclib malonate characterized by an X-ray powder diffraction pattern comprising one or more peaks selected from 6.36°±0.2° 2θ, 13.88°±0.2° 20, 7.31°±0.2° 2θ, 9.34°±0.2° 2θ, 10.05°±0.2° 2θ, 13.59°±0.2° 2θ, 14.08°±0.2° 2θ, 15.21°±0.2° 2θ, 15.67°±0.2° 2θ, 17.53°±0.2° 2θ, 18.70°±0.2° 2θ, 18.98°±0.2° 2θ, 19.38°±0.2° 2θ, 19.67°±0.2° 2θ, 20.16°±0.2° 2θ, 20.39°±0.2° 2θ, 21.01°±0.2° 2θ, 22.27°±0.2° 2θ, 23.35°±0.2° 2θ, 24.15°±0.2° 2θ, 24.67°±0.2° 2θ, 25.00°±0.2° 2θ, 25.18°±0.2° 2θ, 25.57°±0.2° 2θ, 25.93°±0.2° 2θ, 26.21°±0.2° 2θ, 27.19°±0.2° 2θ, and 27.38°±0.2° 2θ.

Clause 123. The formulation of clauses 121 or 122, wherein the crystal form is a crystalline anhydrate.

Clause 124. The formulation of clauses 121 or 122, wherein the crystal form is a crystalline hydrate.

Clause 125. A method of treating a disease or disorder in a subject, the method comprising administering to the subject a therapeutically effective of a formulation of any one of clauses 101 to 124.

Clause 126. A method of treating a disease or disorder in a subject, the method comprising administering to the subject a therapeutically effective of a formulation comprising between about 15% to 35% w/w voruciclib malonate, about 5% to 37% w/w microcrystalline cellulose, about 1% to about 48% w/w lactose monohydrate, about 20% to about 70% w/w dibasic calcium phosphate dihydrate, about 0.1% to about 15% w/w sodium bicarbonate, about 1% to about 20% w/w sodium starch glycolate, and about 0.01% to about 10% w/w magnesium stearate.

Clause 127. The method of clause 126, wherein the voruciclib malonate comprises a crystal form of voruciclib malonate characterized by an X-ray powder diffraction pattern comprising one or more peaks selected from 7.30°±0.2°, 13.58°±0.2°, 14.06°±0.2°, 15.18°±0.2°, 15.66°±0.2°, 17.50°±0.2°, 18.94°±0.2°, 19.54°±0.2°, 22.22°±0.2°, 23.38°±0.2°, 24.10°±0.2°, 24.98°±0.2°, 25.94°±0.2°, 27.26°±0.2°, 28.50°±0.2°, and 32.82°±0.2° 2θ.

Clause 128. The method of clause 126, wherein the voruciclib malonate comprises a crystal form of voruciclib malonate characterized by an X-ray powder diffraction pattern comprising one or more peaks selected from 6.36°±0.2° 2θ, 13.88°±0.2° 2θ, 7.31°±0.2° 20, 9.34°±0.2° 2θ, 10.05°±0.2° 2θ, 13.59°±0.2° 2θ, 14.08°±0.2° 2θ, 15.21°±0.2° 2θ, 15.67°±0.2° 2θ, 17.53°±0.2° 2θ, 18.70°±0.2° 2θ, 18.98°±0.2° 2θ, 19.38°±0.2° 2θ, 19.67°±0.2° 2θ, 20.16°±0.2° 2θ, 20.39°±0.2° 2θ, 21.01°±0.2° 2θ, 22.27°±0.2° 2θ, 23.35°±0.2° 2θ, 24.15°±0.2° 2θ, 24.67°±0.2° 2θ, 25.00°±0.2° 2θ, 25.18°±0.2° 2θ, 25.57°±0.2° 2θ, 25.93°±0.2° 2θ, 26.21°±0.2° 2θ, 27.19°±0.2° 2θ, and 27.38°±0.2° 2θ.

Clause 129. The method of any one of clauses 126 to 128, wherein the formulation comprises about 0.01% to about 10% w/w colloidal silicon dioxide.

Clause 130. The method of any one of clauses 126 to 129, wherein the formulation is comprised into a tablet and the tablet is coated with a film coating.

Clause 131. The method of any one of clauses 125 to 130, wherein the disease or disorder is a blood cancer.

Clause 132a. The method of clause 131, wherein the blood cancer is selected from acute myeloid leukemia (AML), chronic myeloid leukemia (CML), acute lymphocytic lymphoma (ALL), and chronic lymphocytic leukemia (CLL). Clause 132b. The method of clause 131, wherein the blood cancer is a leukemia selected from acute myeloid leukemia (AML), chronic myeloid leukemia (CML), acute lymphocytic lymphoma (ALL), and chronic lymphocytic leukemia (CLL). Clause 132c. The method of clause 131, wherein the blood cancer is a non-Hodgkin lymphoma, such as B-cell or T-cell lymphoma, wherein a B-cell lymphoma is selected from diffuse large B-cell lymphoma (DLBCL), primary mediastinal B-cell lymphoma, intravascular large B-cell lymphoma, follicular lymphoma, small lymphocytic lymphoma (SLL), mantle cell lymphoma, marginal zone B-cell lymphomas, extranodal marginal zone B-cell lymphomas, nodal marginal zone B-cell lymphoma, splenic marginal zone B-cell lymphoma. Burkitt lymphoma, lymphoplasmacytic lymphoma, and primary central nervous system lymphoma. Clause 132d. The method of clause 131, wherein the blood cancer is a T-cell lymphoma selected from precursor T-lymphoblastic lymphoma, peripheral T-cell lymphomas, cutaneous T-cell lymphomas, adult T-cell lymphoma with subtypes: smoldering chronic, acute, and lymphoma, angioimmunoblastic T-cell lymphoma, extranodal natural killer/T-cell lymphoma, nasal type, enteropathy-associated intestinal T-cell lymphoma (EATL) with subtypes I and II, and anaplastic large cell lymphoma (ALCL).

Clause 133. The method of any one of clauses 125 to 132, wherein the formulation is administered to the subject such that the subject receives a daily voruciclib dose between about 50 mg and about 100 mg, between about 100 mg and about 150 mg, between about 150 mg and about 200 mg, between about 200 mg and about 250 mg, between about 250 mg and about 300 mg, between about 300 mg and about 350 mg, between about 350 mg and about 400 mg, between about 400 mg and about 450 mg, between about 450 mg and about 500 mg, between about 500 mg and about 550 mg, between about 550 mg and about 600 mg, between about 600 mg and about 650 mg, between about 650 mg and about 700 mg, between about 700 mg and about 750 mg, between about 750 mg and about 800 mg, between about 800 mg and about 850 mg, between about 850 mg and about 900 mg, between about 900 mg and about 950 mg, or between about 950 mg and about 1,000 mg.

Clause 134. The method of any one of clauses 125 to 132, wherein the formulation is administered to the subject such that the subject receives a daily voruciclib dose of about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1,000 mg.

Clause 135. The method of any one of clauses 125 to 132, wherein the formulation is administered to the subject such that the subject receives a daily voruciclib dose of about 200 mg or about 250 mg.

Clause 136. The method of any one of clauses 125 to 132, wherein the formulation is administered to the subject such that the subject receives a daily voruciclib dose not exceeding 350 mg.

Clause 137. The method of any one of clauses 133 to 136, wherein the voruciclib dose is a voruciclib free base dose.

Clause 138. The method of any one of clauses 125 to 137, wherein the formulation is administered to the subject daily for about one day, about two days, about three days, about 4 days, about 5 days, about 6 days, about 7 days, about 8 days, about 9 days, about 10 days, about 11 days, about 12 days, about 13 days, or about 14 days.

Clause 139. The method of any one of clauses 125 to 137, wherein the formulation is administered to the subject daily for about one week, about two weeks, about three weeks, or about 4 weeks.

Clause 140. The method of any one of clauses 125 to 139, wherein administration of the formulation is paused for about one day, about two days, about three days, about 4 days, about 5 days, about 6 days, about 7 days, about 8 days, about 9 days, about 10 days, about 11 days, about 12 days, about 13 days, or about 14 days.

Clause 141. The method of any one of clauses 125 to 139, wherein administration of the formulation is paused for about one week, about two weeks, about three weeks, or about 4 weeks.

Clause 142. The method of any one of clauses 125 to 141, wherein the formulation is administered to the subject on a 14 days on/14 days off schedule.

Clause 143. The method of any one of clauses 125 to 142, wherein the formulation is administered for about one month, about two months, about three months, about 4 months, about 5 months, about 6 months, about 7 months, about 8 months, about 9 months, about 10 months, about 11 months, or about 12 months.

Clause 144. The method of any one of clauses 125 to 143, wherein the formulation is administered in combination with a BCL-2 inhibitor.

Clause 145. The method of clause 144, wherein the BCL-2 inhibitor is selected from navitoclax, venetoclax, A-1155463, A-1331852, ABT-737, obatoclax, S44563, TW-37, A-1210477, AT101, HA14-1, BAM7, sabutoclax, UMI-77, gambogic acid, maritoclax, MIMI, methylprednisolone, iMAC2, Bax inhibitor peptide V5, Bax inhibitor peptide P5, Bax channel blocker, ARRY 520 trifluoroacetate, or a pharmaceutically acceptable salt of any one thereof.

Clause 146. The method of clause 144, wherein the BCL-2 inhibitor is venetoclax or a pharmaceutically acceptable salt thereof.

Clause 147. The method of any one of clauses 144 to 146, wherein the BCL-2 inhibitor is administered to the subject daily for about one day, about two days, about three days, about 4 days, about 5 days, about 6 days, about 7 days, about 8 days, about 9 days, about 10 days, about 11 days, about 12 days, about 13 days, or about 14 days.

Clause 148. The method of any one of clauses 144 to 146, wherein the BCL-2 inhibitor is administered to the subject daily for about one week, about two weeks, about three weeks, or about 4 weeks.

Clause 149. The method of any one of clauses 144 to 148, wherein administration of the BCL-2 inhibitor is paused for about one day, about two days, about three days, about 4 days, about 5 days, about 6 days, about 7 days, about 8 days, about 9 days, about 10 days, about 11 days, about 12 days, about 13 days, or about 14 days.

Clause 150. The method of any one of clauses 144 to 148, wherein administration of the BCL-2 inhibitor is paused for about one week, about two weeks, about three weeks, or about 4 weeks.

Clause 151. The method of any one of clauses 144 to 150, wherein the BCL-2 inhibitor is administered to the subject on a 14 days on/14 days off schedule.

Clause 152. The method of any one of clauses 144 to 151, wherein the BCL-2 inhibitor is administered to the subject for about one month, about two months, about three months, about 4 months, about 5 months, about 6 months, about 7 months, about 8 months, about 9 months, about 10 months, about 11 months, or about 12 months.

### EXAMPLES

### Example 1: Monotherapy Phase 1 Studies in Solid Tumors

2 weeks on/1 week off schedule: 75 to 850 mg; 29 pts in dose escalation/expansion at 600 mg cohorts; 41% disease control rate; 1 PR and 8 SD lasting 2 to 6 months.

Daily continuously schedule: 75 to 500 mg; 39 pts in dose escalation/expansion at 350 mg cohorts; 31% disease control rate; 12 SD lasting a median of 15 weeks.

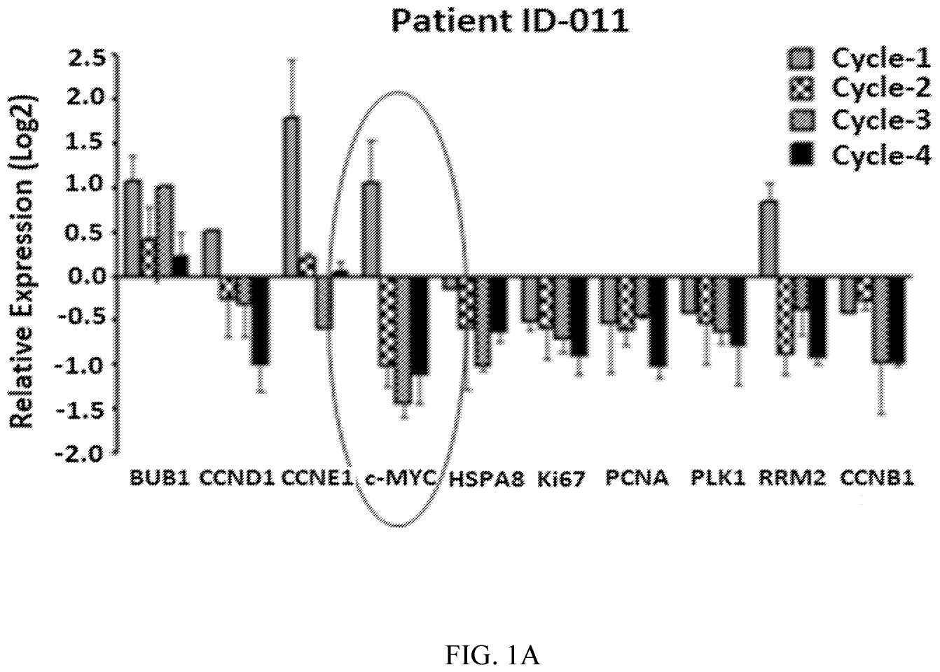

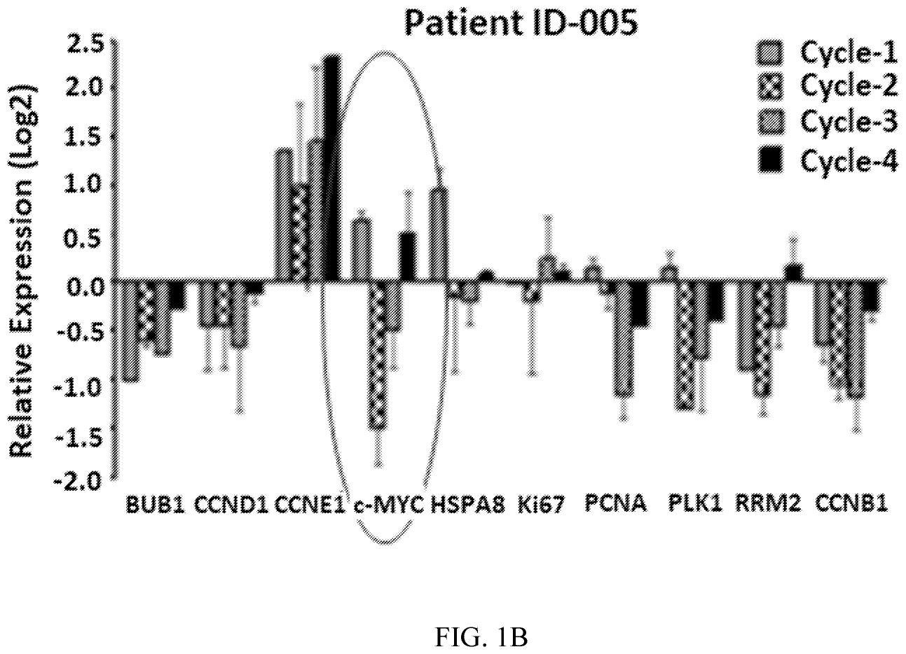

Safety profile: most common AEs involved GI tract; no evidence of myelosuppression Decreased c-MYC Expression in Solid Tumors: 10 gene biomarkers evaluated in Phase 1 daily dosing study; c-MYC expression decreased in 17/25 patients (68%) tested (FIGS. 1A and 1B).

### Example 2: Study of Voruciclib+Vemurafenib in BRAF-mut Advanced/Inoperable Malignant Melanoma

Voruciclib 150 mg daily plus vemurafenib 720 mg or 960 mg BID in 28-day cycles; 9 pts treated before study termination; 8 patients evaluable for efficacy; 5 patients were BRAFi refractory, response=PD; 3 patients were BRAF/MEK naïve, 1 CR and 2 PR ongoing for 3 to 14 months; most common AEs were fatigue, constipation, diarrhea, arthralgia and headache; 1 DLT=grade 3 fatigue.

### Example 3: CR in a Patient with Pulmonary Metastases

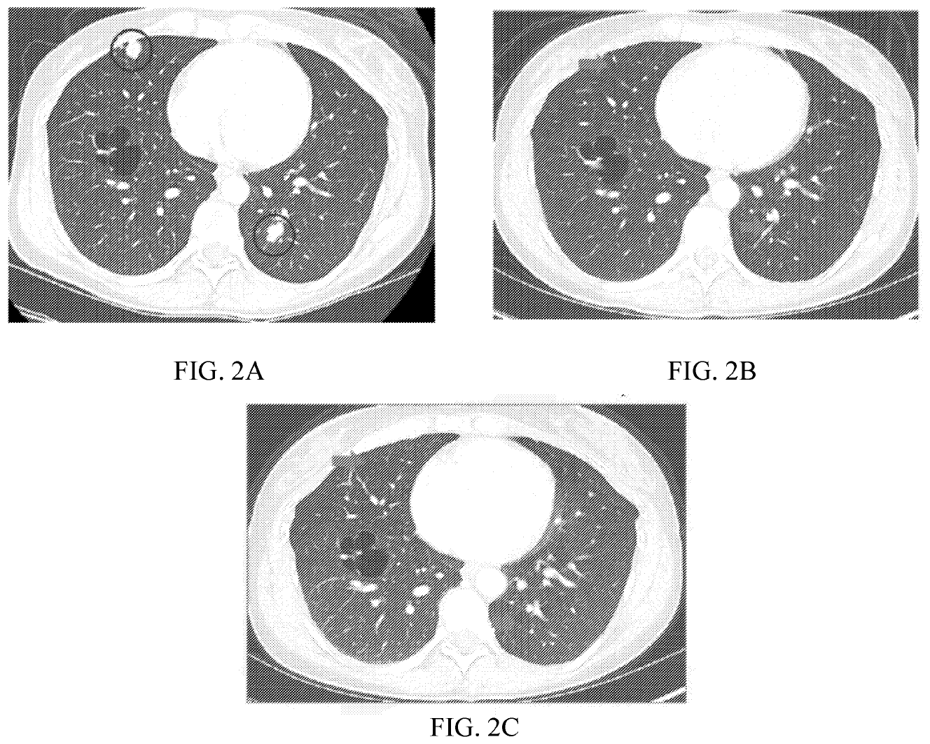

FIG. 2A-2C illustrate CR in a Patient with Pulmonary Metastases; FIG. 2A: baseline CT scan; FIG. 2B: 2 months after starting the trial, radiological CR based on official radiological report; FIG. 2C: 14 months after starting the trail, patient remained on trial for 12 months only, and CR remained durable for 14 months.

### Example 4: Leveraging CDK9 Regulation of MCL1: Phase 1 Study in R/R B-Cell Malignancies and AML

Study population: Relapsed/Refractory B-cell malignancies; Relapsed/Refractory AML; Dose escalation with standard 3+3 design.

Endpoints: Safety and tolerability, Pharmacokinetics, Biologic correlative studies (BH3 profiling, MCL-1 expression, molecular mutations analysis), Response rates.

Voruciclib single agent dose escalation: 50 mg>100 mg>150 mg>200 mg.

Phase I Study in Hematologic Malignancies: 24 pts treated in 3 dose levels, 10 AML and 14 B-cell malignancies, No GI toxicity or neutropenia at doses studied, Favorable PK profile across all voruciclib studies (Half-life 24-28 hours supports once-a-day dosing, Dose proportional Cmax and AUC; High volume of distribution indicates broad entry into tissues); Doses of 150-200 mg may achieve plasma concentrations sufficient to inhibit molecular target.

### Example 5: Voruciclib Shows Preferential Tumor Accumulation in Preclinical Model

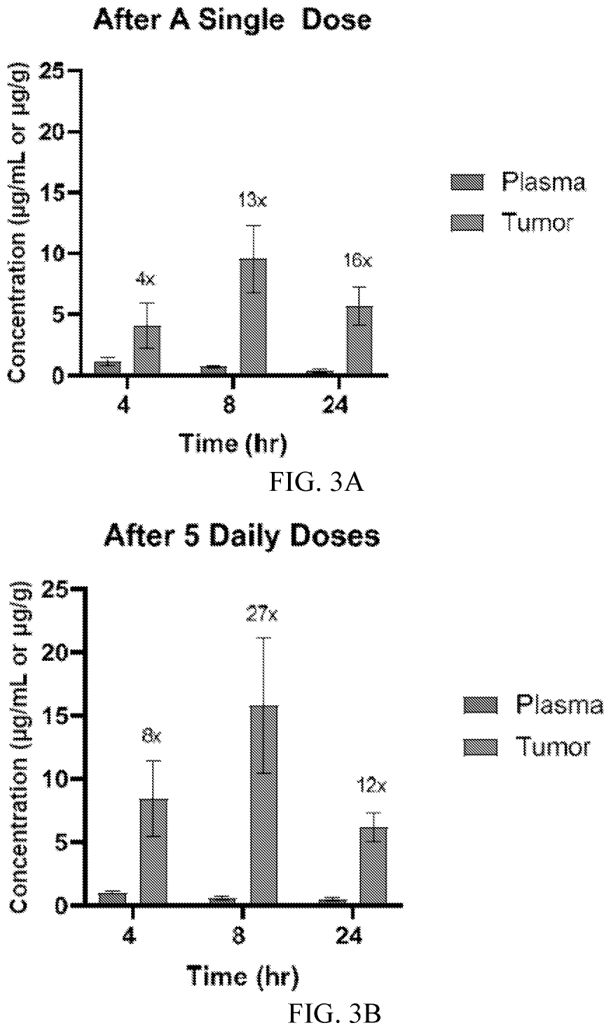

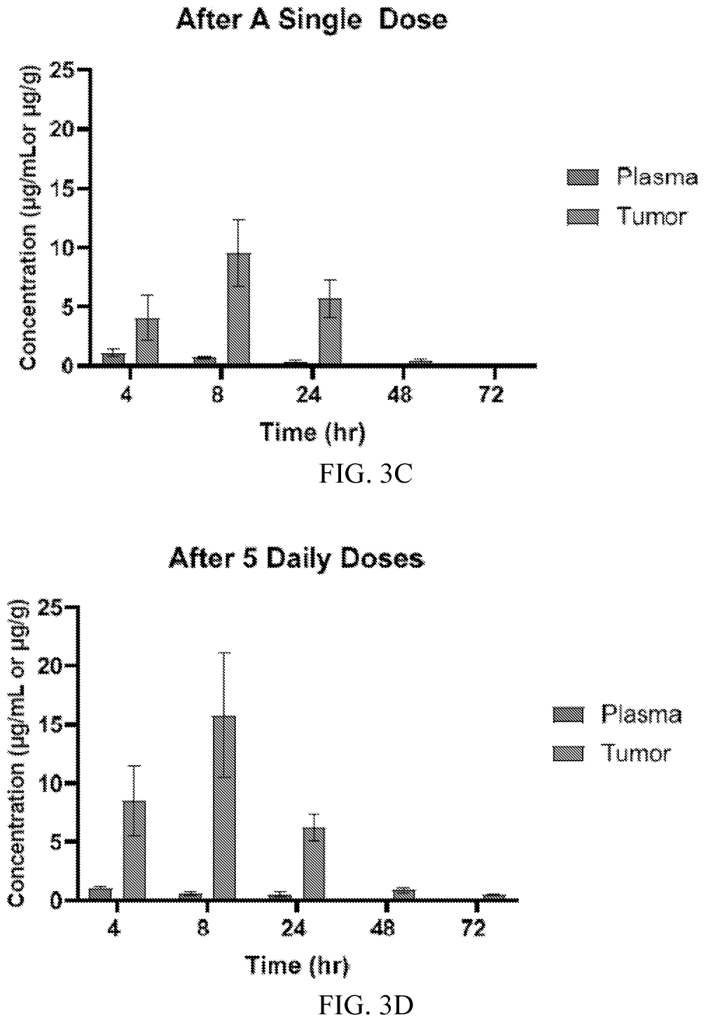

Voruciclib Shows Preferential Tumor Accumulation in Preclinical Model (FIGS. 3A-3D). HCT-116 CRC cell xenograft in SCID mice. 8 mice per time point (2 control, 6 orally dosed with voruciclib at 100 mpk). Animals were randomized into 2 groups when tumors reached 100 mm diameter. Group A assigned single dosing. Group B assigned 5 day dosing. 8 mice per time point (2 control, 6 orally dosed with voruciclib at 100 mpk). Drug concentration was measured in tumor and plasma after last dose at the following time points: 0, 4, 8, 24, 48, 72 hrs. The accumulation index of voruciclib in tumors after 5 days of repeat dosing was 1.45. Concentration of voruciclib in plasma is in units of μg/ml and in μg/g for tumors. Voruciclib fold increase in tumors relative to plasma are indicated.

Higher levels (>5 fold) of voruciclib were found in tumor at 8 hours compared to 24 hours post dosing. Negligible levels of voruciclib were observed in the plasma at 24 hours. Moreover, tumor to plasma ratio was found to be >5 fold at both time points.

### Example 6: Evidence of Biologic Activity in AML

Differentiation syndrome seen in 5 pts (50%) (Increased WBC without increased in blasts, bone pain, pulmonary symptoms; Response to corticosteroids).

Differentiation syndrome with ATRA. IDHi, and other AML targeted therapies.

### Example 7: Voruciclib Synergizes with Venetoclax in Venetoclax Sensitive and Resistant Cell Lines

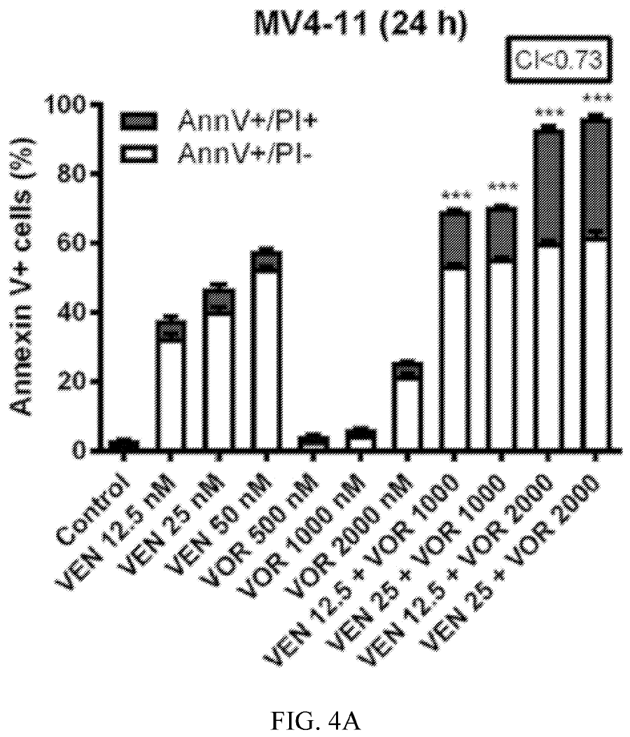

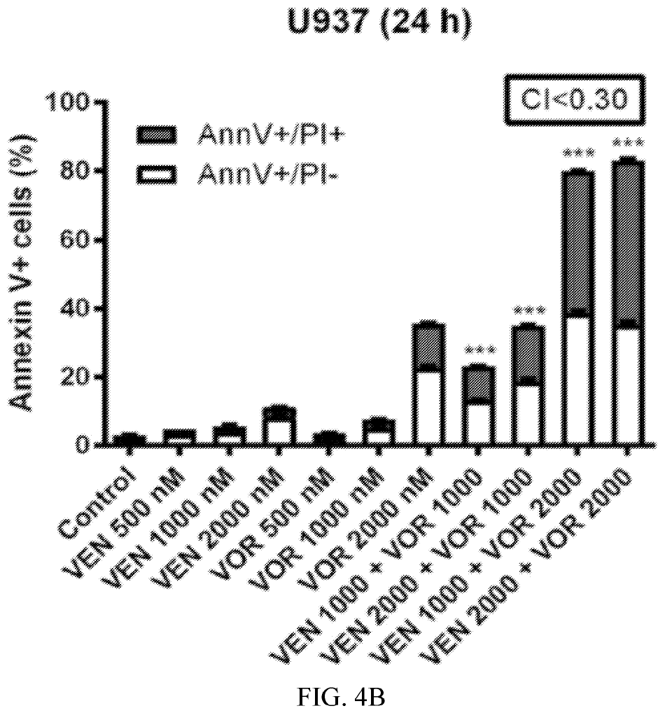

FIGS. 4A and 4B illustrate that Voruciclib Synergizes with Venetoclax in Venetoclax Sensitive and Resistant Cell Lines. FIG. 4A: Ven Sensitive; FIG. 4B: Ven Resistant.

Phase 1 Study of Voruciclib+Venetoclax in AML; Study population: Relapsed/Refractory B-cell malignancies; Relapsed/Refractory AML; Dose escalation with standard 3+3 design.

Endpoints: Safety and tolerability; Pharmacokinetics; Biologic correlative studies (BH3 profiling, MCL-1 expression, Molecular mutations analysis); Response rates.

Voruciclib single agent dose escalation: 50 mg>100 mg>150 mg>200 mg

Voruciclib+Venetoclax dose escalation: 100 mg>150 mg>200 mg,

### Example 8: Voruciclib Dosing Regimen

PK data for 2 weeks of daily dosing followed by 1 week without dosing in a 21-day cycle is presented in Table 1, and PK data for daily dosing continuously is presented in Table 2.

TABLE 1
Descriptive Statistics (Mean ± Std. Dev) of PK Parameters of Voruciclib on Day 1 and Day 13

| | | Dose | | | | |
|---|---|---|---|---|---|---|
| | | 75 mg | 150 mg | 300 mg | 600 mg | 850 mg |
| Parameter | Day | (n = 3) | (n = 3) | (n = 3) | (n = 16) | (n = 4) |
| Tₘₐₓ(h)* | Day 1 | 4.000 | 4.000 | 6.000 | 4.000 | 9.000 |
| | | (2.000-4.000) | (2.000-12.000) | (4.000-12.000) | (2.000-24.000) | (4.000-12.000) |
| | Day 13 | 4.000 | 4.000 | 12.000 | 4.000 | 5.000 |
| | | (2.000-6.000) | (4.000-6.000) | (4.000-12.000) | (2.000-12.000) | (4.000-6.000) ^{#} |
| Cₘₐₓ (ng/mL) | Day 1 | 193.146 ± | 456.390 ± | 1003.518 ± | 1628.010 ± | 1732.214 ± |
| | | 181.7593 | 160.4270 | 448.0968 | 1242.8176 | 330.5233 |
| | Day 13 | 313.116 ± | 895.925 ± | 2100.265 ± | 2944.112 ± | 4449.010 ± |
| | | 24.5507 | 247.2074 | 639.0394 | 1422.6062 | 111.7476^{#} |
| AUC₀₋ₜ | Day 1 | 2279.320 ± | 6980.121 ± | 15653.965 ± | 24823.697 ± | 31351.031 ± |
| (ng · h/mL) | | 1367.3669 | 2955.5550 | 1346.4779 | 18560.8532 | 5484.4228 |
| | Day 13 | 5286.217 ± | 12957.290 ± | 40419.748 ± | 43508.009 ± | 83939.127 ± |
| | | 423.6548 | 1236.0382 | 10734.1354 | 21192.6452 | 13229.6999^{#} |
| AUC_{0-∞} | Day 1 | 4209.822 ± | 15765.319 ± | 147671.801 ± | 61535.871 ± | 108619.189 ± |
| (ng · h/mL) | | 2206.6963 | 1432.9428^{#} | 167803.1221^{#} | 55710.1558^{€} | 40549.3234^{#} |
| | Day 13 | 8659.785 ± | 28167.439 ± | 59331.780 ± | 86004.296 ± | 170083.523 ± |
| | | 354.5380 | 13275.2871 | — \hat{ } | 62687.4524^{§} | 93983.7693^{#} |
| AUC₀₋₂₄ | Day 1 | 2279.320 ± | 6980.121 ± | 15653.965 ± | 24823.697 ± | 31351.031 ± |
| (ng · h/mL) | | 1367.3669 | 2955.5550 | 1346.4779 | 18560.8532 | 5484.4228 |
| | Day 13 | 5286.217 ± | 12957.290 ± | 40419.748 ± | 43508.009 ± | 83939.127 ± |
| | | 423.6548 | 1236.0382 | 10734.1354 | 21192.6452 | 13229.6999^{#} |
| t½ (h) | Day 1 | 21.658 ± | 32.527 ± | 137.821 ± | 32.544 ± | 50.044 ± |
| | | 13.4942 | 21.8661^{#} | 173.8172^{#} | 37.9895^{€} | 10.1297^{#} |
| | Day 13 | 16.928 ± | 26.061 ± | 16.166 ± | 19.036 ± | 22.380 ± |
| | | 0.9072 | 19.9929 | —\hat{ } | 14.8429^{§} | 14.2008^{#} |
| Vz/F (L) | Day 1 | 1279.160 ± | 1653.836 ± | 3978.953 ± | 1552.707 ± | 2151.883 ± |
| | | 833.7216 | 1757.3463^{#} | 5096.6355^{#} | 1962.2874^{€} | 162.8208^{#} |
| | Day 13 | 348.886 ± | 458.679 ± | 185.110 ± | 393.745 ± | 314.518 ± |
| | | 44.8521 | 393.3117 | —\hat{ } | 255.5617^{§} | 157.8920^{#} |
| Cl/F (L/h) | Day 1 | 42.217 ± | 25.301 ± | 19.255 ± | 36.935 ± | 27.881 ± |
| | | 24.7244 | 13.8587^{#} | 1.5779 | 29.4853 | 5.8668 |
| | Day 13 | 14.247 ± | 11.649 ± | 7.762 ± | 24.177 ± | 10.254 ± |
| | | 1.1088 | 1.1334 | 1.9348 | 33.4293 | 1.6161^{#} |
| Accumulation | Day 13/ | 2.937 ± | 2.129 ± | 2.602 ± | 2.064 ± | 3.139 ± |
| Ratio | Day 1 | 1.5793 | 0.9716 | 0.7871 | 0.9112 | 1.2489^{#} |
| (AUC_{0-24 h}) | | | | | | |

*Median (Range) values were reported for Tₘₐₓ.
\hat{ }n = 1, ^{#}n = 2, ^{€}n = 11 and ^{§}n = 15
Since there were inadequate time-points to characterize the elimination phase AUC_{0-∞}, t½ and Vz/F could not be computed for patients 106 (Day 1), 202 (Day 1), 204 (Day 1), 216 (Day 13), 302 (Day 1), 306 (Day 1), 409 (Day 13), 410 (Day 1), 418 (Day 1), 419 (Day 1), 501(Day 1) and 501(Day 13).

TABLE 2
Descriptive Statistics (Mean ± Std. Dev) of PK Parameters of Voruciclib on Day 1 and Day 15

| | | Dose | | | | |
|---|---|---|---|---|---|---|
| | | 75 mg | 150 mg | 250 mg | 350 mg | 500 mg |
| Parameter | Day | (n = 3) | (n = 3) | (n = 3) | (n = 24) | (n = 6) |
| Tmax(h)* | Day 1 | 4.000 | 6.000 | 4.000 | 6.000 | 6.000 |
| | | (4.000-6.000) | (4.000-12.000) | (2.000-6.000) | (1.000-12.000) | (2.000-24.000) |
| | Day 15 | 2.000 | 6.000 | 4.000 | 4.000 | 2.000 |
| | | (0.000-2.000) | (0.250-24.000) | (2.000-6.000) | (0.500-24.000) ^{§} | (1.000-6.000) ^{##} |
| Cmax | Day 1 | 125.116 ± | 257.918 ± | 471.955 ± | 623.486 ± | 1135.913 ± |
| (ng/mL) | | 76.1007 | 48.9739 | 176.8485 | 147.4429 | 502.3381 |
| | Day 15 | 331.174 ± | 754.927 ± | 1047.832 ± | 1493.904 ± | 2254.686 ± |
| | | 287.8394 | 119.5624 | 268.6660 | 537.5295^{§} | 1259.5916^{##} |
| AUC0-t | Day 1 | 1726.805 ± | 4852.919 ± | 6114.663 ± | 10395.509 ± | 16370.472 ± |
| (ng · h/mL) | | 929.4239 | 1101.4055 | 2456.5442 | 3024.9074 | 6058.2118 |
| | Day 15 | 5567.102 ± | 16642.661 ± | 18537.142 ± | 26733.146 ± | 40465.337 ± |
| | | 5258.2498 | 2969.0833 | 6973.9509 | 7478.4641^{§} | 25259.1489^{##} |
| AUC0-∞ | Day 1 | 3424.383 ± | 15592.890 ± | 16383.699 ± | 133647.705 ± | 41032.540 ± |
| (ng · h/mL) | | 1737.4934 | 7140.1056^{#} | 7564.4777 | 342360.8544^{§} | 18507.8312^{†} |
| | Day 15 | 27200.362 ± | 164073.250 ± | 54524.054 ± | 164113.625 ± | 108301.704 ± |
| | | 14338.0479 | — \hat{ } | 2108.8817^{#} | 236753.6624^{€} | 71929.6562^{†} |
| AUC0-24 | Day 1 | 1726.805 ± | 4852.919 ± | 6114.663 ± | 10388.095 ± | 16370.472 ± |
| (ng · h/mL) | | 929.4239 | 1101.4055 | 2456.5442 | 3026.1945 | 6058.2118 |
| | Day 15 | 5567.102 ± | 16642.661 ± | 18537.142 ± | 26733.146 ± | 40465.337 ± |
| | | 5258.2498 | 2969.0833 | 6973.9509 | 7478.4641^{§} | 25259.1489^{##} |
| t½ (h) | Day 1 | 21.776 ± | 39.589 ± | 34.397 ± | 129.341 ± | 24.798 ± |
| | | 6.8124 | 9.6927^{#} | 21.5177 | 308.5582^{§} | 5.2455^{†} |
| | Day 15 | 358.828 ± | 131.733 ± | 38.750 ± | 72.548 ± | 42.366 ± |
| | | 559.9488 | — \hat{ } | 18.5958^{#} | 86.4263^{€} | 25.7296^{†} |
| Vz/F (L) | Day 1 | 860.496 ± | 579.369 ± | 765.765 ± | 155.214 ± | 72.515 ± |
| | | 526.4529 | 130.7782^{#} | 461.4432 | 112.0200^{§} | 25.3428^{†} |
| | Day 15 | 981.869 ± | 173.749 ± | 258.903 ± | 77.571 ± | 51.024 ± |
| | | 1326.5232 | — \hat{ } | 133.0243^{#} | 80.6852^{€} | 24.6115^{†} |
| CL/F | Day 1 | 28.856 ± | 10.746 ± | 17.724 ± | 4.224 ± | 2.107 ± |
| (L/h) | | 20.5929 | 4.9209^{#} | 8.3061 | 3.7491^{§} | 0.8559^{†} |
| | Day 15 | 3.264 ± | 0.914 ± | 4.589 ± | 1.607 ± | 1.031 ± |
| | | 1.4856 | — \hat{ } | 0.1775^{#} | 2.0264^{€} | 0.7311^{†} |
| Accumulation | Day 15/ | 2.634 ± | 3.637 ± | 3.090 ± | 2.767 ± | 2.577 ± |
| Ratio (AUC0-t) | Day 1 | 1.6433 | 1.3888 | 0.4162 | 1.0640^{§} | 0.7557^{##} |

*Median (Range) values were reported for Tmax.
\hat{ } n = 1, ^{#}n = 2, ^{†}n = 4, ^{##}n = 5, ^{€}n = 13 and ^{§}n = 18
Since there were inadequate time-points to characterize the elimination phase AUC0-□, t½ and Vz/F could not be computed for patients 004 (Day 1), 004 (Day 15), 009 (Day 15), 011 (Day 1), 011 (Day 15), 023 (Day 1), 024 (Day 1), 026 (Day 1), 031 (Day 1), 005 (Day 15), 031 (Day 15), 032 (Day 15), 035 (Day 1), 035 (Day 15), 036 (Day 15), 013(Day 1), 13 ay 15) and 014 (Day 1).

Voruciclib half-life is designated as t1/2 in the tables (expressed in hours). Steady state (i.e., Day 13-15) half-life ranges from 16 hours to 358 hours, with an average of 24 to 48 hours. There is interpatient variability, explaining the outlier values. When the drug is stopped, it takes ˜5 half-lives, or 5-10 days for the drug to be eliminated from the plasma.

Voruciclib volume of distribution is designated as Vz/F (expressed in liters). Steady state (i.e., Day 13-15) volume of distribution ranges from 185 L to 982 L, with an average of ˜300 L (and outlier values). Blood volume is ˜5 L, and thus, in some embodiments, voruciclib volume of distribution is ˜60 times larger than the blood volume, indicating broad distribution into tissues. When the drug is stopped it takes an additional 3 days to clear from the tissue after it clears the plasma.

Without wishing to be bound by any particular theory, it is believed that by using a 14 days on therapy followed by 14 days off therapy, there is enough time for the drug to be eliminated from the plasma (Day 19 to Day 24) and another 3 days (Day 22 to Day 27) to be eliminated for the tissues, thereby preventing accumulation into tissue with continuous daily dosing, and potential toxicities. Without wishing to be bound by any particular theory, it is believed that voruciclib dosing on a 14 days on/14 days off schedule can prevent tissue toxicity. In some embodiments, such dosing regimen can match the dosing schedule of a combination drug.

### Example 9: Summary of Formulation Development for Voruciclib Malonate

Voruciclib can form a gel when dissolved in various media, depending on the other chemicals present. Voruciclib malonate does not form a gel in water or aqueous buffers across the pH range, in contrast to voruciclib hydrochloride. However, during the development of an immediate-release tablet formulation of the malonate salt, tablets were observed to gel at acidic pH (the media used to test dissolution) depending on the excipients that were present in the formulation.

Below summarizes the efforts which led to the discovery of a tablet formulation that does not form a gel during dissolution:
1. Tried to formulate the drug with less water-soluble excipients and various concentrations—No positive effect.
2. Tried to formulate the drug with multiple excipients which are less water soluble and various concentrations—No positive effect.
3. Tried to formulate the drug with a combination of water soluble and less water-soluble excipients and at various concentrations—No positive effect.
4. Tried to formulate with high levels of disintegrants—No positive effect.
5. Tried to add sodium bicarb to formulation and at various concentrations—the thought was adding sodium bicarb would interact with the acid to produce CO2 that would help break apart the tablet. Gelling was reduced or eliminated at higher concentrations of sodium bicarb. Unfortunately, increasing sodium bicarb led to tablet manufacturing problems (poor flow, high tablet weight), and caused the tablets to float during dissolution testing leading to poor dissolution profiles.
6. It was found that flow could be improved by reducing sod bicarb levels and that increasing dical phosphate eliminated the tablet floating which led to improved dissolution profiles while eliminating the gelling issue.
7. It was also found that for certain formulations floating could be eliminated by changing the tablet size, shape and weight.

TABLE 3
Formulation of the disclosure

| Ingredient | % w/w | mg/tablet |
|---|---|---|
| Voruciclib Malonate | 23.49 | 61.07 |
| Avicel pH 102 | 19.23 | 50.00 |
| Lactose Monohydrate 316 Fast Flo | 15.55 | 40.43 |
| Dibasic calcium Phosphate | 32.69 | 85.00 |
| Sodium Bicarbonate, NF | 4.00 | 10.40 |
| Sodium Starch Glycolate | 4.04 | 10.50 |
| Magnesium Stearate | 1.00 | 2.60 |

Excipient compatibility studies were done by making binary mixtures of voruciclib malonate with the excipients listed in Table 4. These binary (drug:excipient, 1:1) mixtures were stored at 40° C./75% relative humidity (RH) and tested for assay and related substances at 0, 2, and 4 weeks.

TABLE 4
Excipients Evaluated for Compatibility with Voruciclib Malonate

| Excipient | Function |
|---|---|
| Lactose Monohydrate | Diluent |
| Microcrystalline cellulose | Diluent |
| Silicified microcrystalline cellulose | Diluent |
| Mannitol | Diluent |
| Dicalcium Phosphate | Diluent |
| Croscarmellose sodium | Disintegrant |
| Sodium Starch Glycolate | Disintegrant |
| Magnesium Stearate | Lubricant |
| Steric Acid | Lubricant |
| Colloidal Silicon dioxide | Flow agent |
| Crospovidone | Binder |
| Povidone | Binder |
| Hydroxypropyl methylcellulose (HPMC) | Binder |
| Sodium Lauryl Sulfate | Surfactant |
| Poloxamer | Surfactant |
| Polyethylene Glycol (PEG) | Solubilizer |
| Propylene Glycol | Solubilizer |

Based on the excipient compatibility study results, the excipients listed in Table 4 were selected for an initial formulation screening study. It was previously observed that voruciclib and its salts can gel upon hydration of the drug, and this gelling can have an impact on the disintegration and dissolution of voruciclib from pharmaceutical preparations. The excipients for the tablet formulation were selected based on their compatibility with voruciclib malonate as well as their ability to aid in the manufacture of voruciclib malonate tablets and reduce the gelling of voruciclib malonate tablets.

An initial prototype formulation was developed that did not contain sodium bicarbonate and it was found that disintegration and dissolution (in 0.1 N HCl media) were not acceptable due to gelling for this initial formulation. Attempts to modify the formulation evaluated the effect of changing microcrystalline cellulose, lactose, dibasic calcium phosphate, and sodium starch glycolate levels to overcome the observed gelling issue. These changes led to only minor improvements in the gelling issue. Sodium bicarbonate was then added to the formulation to aid in the disintegration of the voruciclib malonate tablets, thereby reducing the impact of the gelation phenomenon on the dissolution testing.

The development approach was to use a common granule approach and to manufacture different tablet strengths by adjusting total tablet weight (Table 5).

TABLE 5
Excipients and Ranges used in Voruciclib
Malonate Tablets Formulation Development

| | | Range tested |
|---|---|---|
| Excipient | Function | (% w/w) |
| Voruciclib Malonate^{a} | API | 20.36-30.54 |
| Microcrystalline Cellulose | Diluent | 14.32-37.00 |
| (PH-102) | | |
| Lactose Monohydrate | Diluent | 8.55-46.46 |
| Dibasic Calcium Phosphate | Diluent | 0-43.32 |
| Dihydrate | | |
| Sodium Bicarbonate | Aid in disintegration | 0-6.92 |
| Sodium Starch Glycolate | Disintegrant | 3.5-8.00 |
| Magnesium Stearate | Lubricant | 0.87-1.00 |

^{a}Exact amount is corrected for drug substance assay (free base on the anhydrous, solvent free basis)

Based on the results of the formulation development study, the formulation listed in Table 6 was selected and scaled up to a batch size of approximately 0.3 kg.

TABLE 6
Voruciclib Malonate Development Tablet
Composition (Batch Size 0.3 kg)

50 mg Tablet

| Ingredient | mg per tablet | % w/w |
|---|---|---|
| Voruciclib Malonate^{a} | 61.07 | 21.81 |
| Microcrystalline Cellulose (PH-102) | 40.10 | 14.32 |
| Lactose Monohydrate | 26.73 | 9.55 |
| Dibasic Calcium Phosphate Dihydrate | 121.30 | 43.32 |
| Sodium Bicarbonate | 11.20 | 4.00 |
| Sodium Starch Glycolate | 16.80 | 6.00 |
| Magnesium Stearate | 2.80 | 1.00 |
| Total Tablet weight | 280.0 | N/A |

^{a}Exact amount is corrected for drug substance assay (free base on the anhydrous, solvent free basis). Salt conversion factor is 0.8187 so 61.07 mg of voruciclib malonate equals 50 mg of voruciclib free base

The formulation listed in Table 6 performed as expected (e.g., gelling was no longer an issue and rapid dissolution was observed) with respect to disintegration and dissolution, but the flow of the blend needed to be improved to ensure the manufacturability of the formulation. To improve the flow Colloidal Silicon Dioxide (1.0%) was added to the formulation and yielded the final formulation intended for use in clinical trials and listed in Table 7.

The final core tablets were coated with a non-functional coat of 3% w/w of Opadry II as indicated in Table 7.

TABLE 7
Formulations of the disclosure

| | 50 mg^{a} Tablet | 100 mg^{a} Tablet | |
|---|---|---|---|
| Ingredient | mg per Tablet | mg per Tablet | % w/w |
| Voruciclib Malonate | 61.07 | 122.14 | 21.81 |
| Microcrystalline Cellulose | 40.10 | 80.20 | 14.32 |
| (PH-102) | | | |
| Lactose Monohydrate | 23.93 | 47.86 | 8.55 |
| Dibasic Calcium Phosphate | 121.30 | 242.60 | 43.32 |
| Dihydrate | | | |
| Sodium Bicarbonate | 11.20 | 22.40 | 4.00 |
| Sodium Starch Glycolate | 16.80 | 33.60 | 6.00 |
| Colloidal Silicon Dioxide | 2.80 | 5.60 | 1.00 |
| Magnesium Stearate | 2.80 | 5.60 | 1.00 |
| Total tablet Weight | 280.0 | 560.0 | 100.0 |
| (Core Tablet) | | | |
| Opadry II 85F18422 White | 8.4 | 16.8 | 3.0 |
| Total tablet | 288.4 | 576.8 | N/A |
| (Film coated tablet) | | | |

^{a}Exact amount is corrected for drug substance assay (free base on the anhydrous, solvent free basis). Salt conversion factor is 0.8187 so 61.07 mg of voruciclib malonate equals 50 mg of voruciclib free base and 122.14 mg of voruciclib malonate equals 100 mg of voruciclib free base.

The manufacturing process was developed as a simple direct compression tablet blend of voruciclib malonate with dibasic calcium phosphate dihydrate, microcrystalline cellulose, lactose monohydrate, sodium starch glycolate, and sodium bicarbonate. Colloidal silicon dioxide was added to improve the flow of the blend. The blend is lubricated with magnesium stearate. Additional process development will be undertaken as the molecule moves through the development process.

In some embodiments, use of sodium bicarb reduces or eliminates gelling. In some embodiments, use of dicalcium phosphate with sodium bicarb reduces or eliminates gelling. In some embodiments, use of dicalcium phosphate prevents tablets from floating. In some embodiments, the ratio of sodium bicarbonate to dicalcium phosphate modulates the formulation of voruciclib such that it does not gel, does not float, and has a reproducible dissolution profile. In some embodiments, combinations of tablet size, shape, and formulation prevent tablets from floating.

Conventional HDPE bottles, induction sealed liners, polypropylene child-resistant caps, and desiccant canisters were selected as the container closure system for voruciclib malonate tablets. The need for a desiccant will be evaluated as the formulation moves through the development process. The specification for voruciclib malonate tablets, 50 mg and 100 mg, is provided in Table 8.

TABLE 8
Specification for Voruciclib Malonate Tablets, 50 mg and 100 mg

| | Analytical | |
|---|---|---|
| Attribute | Procedure | Acceptance Criteria |
| Appearance | Visual Inspection | White to off-white oval tablets free of |
| | | visible defects. |
| | | Tablet length (inches)^{a}: |
| | | 50 mg: 0.49 ± 0.04 |
| | | 100 mg: 0.64 ± 0.04 |
| Identification^{a} | HPLC | The retention time of the major peak of |
| | | voruciclib malonate tablets is consistent |
| | | with that of the voruciclib malonate |
| | | reference standard |
| Assay | HPLC | 90.0%-110.0% of label claim |
| Uniformity of Dosage Units^{a} | HPLC | Meets Criteria of USP <905> |
| Organic Impurities | | |
| ME-522-i455 | HPLC | NMT 1.0% |
| ME-522-i451 | | NMT 1.0% |
| Any Unspecified Degradation | | NMT 0.5 area % |
| Product^{b} | | |
| Total Degradation Products^{c} | | NMT 5.0 area % |
| Dissolution | USP <711> | Q = 80% at 30 minutes |
| Water Content | USP <921> | Report results |
| Microbial Limits | | |
| Total Aerobic Microbial | USP <61> | NMT 1000 CFU/g |
| Count | | |
| Total Combined Yeasts and | | NMT 100 CFU/g |
| Molds | | |
| Specified Microorganisms: | | |
| Escherichia. coli | USP <62> | Absent in 1 gram |

Abbreviations:
CFU = Colony-forming units;
NMT = Not more than;
USP = United States Pharmacopeia;
HPLC = High performance liquid chromatography;
^{a}Tested at release only
^{b}Largest Unspecified Degradation Product >0.05% area. Peaks designated as Synthetic Impurities at the time of analysis are excluded
^{c}Sum of degradation products ≥0.05% area

Batch analysis data for a development lot of voruciclib malonate tablets, 50 mg, manufactured at laboratory scale are provided in Table 9. The formulation differs from the intended clinical formulation in that it does not contain colloidal silicon dioxide. The manufacturing process for this lot was similar to that described in Example 10, with the exception that the colloidal silicon dioxide was not added during processing.

TABLE 9
Batch Release Data for Voruciclib Malonate
Tablets, 50 mg and 100 mg Engineering Lots

| | | Batch No. | |
|---|---|---|---|
| | | Lot 0000113822 | Lot 0000113823 |
| | | API Input Lot | |
| | | 02MEI06-01-107 | 02MEI06-01-107 |
| | | Strength | |
| | | 50 mg | 100 mg |
| | | Date of Manufacture | |
| | | 8 Nov. 2021 | 9 Nov. 2021 |
| | | Scale (No. of Tablets) | |
| | | 1200 g | 1300 g |
| | | (4286 Tablets) | (2321 Tablets) |
| | | Use | |
| | Acceptance | Stability | Stability |
| Attribute | Criteria^{b} | Results | Results |
| Appearance by Visual | Report Results | Off white oval | Off white oval |
| Inspection | | shaped tablets | shaped tablets |
| Assay by HPLC | 90.0%-110.0% | 98.5% | 100.3% |
| | of label claim | | |
| Uniformity of Dosage | Report Results | Mean 101.7% | Mean 103.4% |
| Units by HPLC^{c} | | AV = 2.9 | AV 3.6 |
| Organic Impurities | | | |
| by HPLC | | | |
| ME-522-i455 | NMT 1.0% area | 0.34% area | 0.33% area |
| ME-522-i451 | NMT 1.0% area | 0.66% area | 0.65% area |
| Any Unspecified | NMT 0.5% area | 0.09% area | 0.08% area |
| Degradation Product^{d} | | | |
| Total Degradation | NMT 5.0% area | 1.3% area | 1.2% area |
| Products^{e} | | | |
| Dissolution by USP | Q = 80% at | 97% | 98% |
| <711> | 30 minutes | Range 94-100% | Range 93-103% |
| Water Content by KF, | Report results | 2.2% w/w | 2.1% w/w |
| USP <921> | | | |

Abbreviations:
HPLC = High performance liquid chromatography;
RRT = Relative Retention Time;
KF = Karl Fischer;
NMT = Not More Than;
USP = United States Pharmacopeia
^{b}Acceptance criteria applied during development
^{c}Tested at release only
^{d}Largest Unspecified Degradation Product ≥0.05% area. Peaks designated as Synthetic Impurities at the time of analysis are excluded.
^{e}Sum of degradation products ≥0.05% area

Batch analysis data for a development lot of voruciclib malonate tablets. 50 mg, manufactured at laboratory scale are provided in Table 10. The formulation is the intended clinical formulation containing colloidal silicon dioxide. The manufacturing process for this lot is described in Example 10.

TABLE 10
Batch Analysis for Voruciclib Malonate Drug Substance, Batch 02MEI06-01-107

| Attribute | Acceptance Criteria | | Results | |
|---|---|---|---|---|
| Appearance by visual | Report Results | | Pale yellow | |
| inspection | | | solid | |
| Identity by NMR | Conforms to Reference Standard | | Conforms | |
| Identification by HPLC | Retention Time corresponds to | | 100% | |
| | Reference Standard (98-102%) | | | |
| Assay of voruciclib malonate | 95.0%-105.0% wt/wt | | 96.9% | |
| by HPLC | | | | |
| Chiral Purity by HPLC | NLT 98.0% | | 100% | |
| Organic Impurities by HPLC | | | Area % | |
| Specified | | | | |
| ME-522-i483 | NMT 1.0 area % | | 0.13% | |
| ME-522-i435 | | | 0.69% | |
| ME-522-i455 | | | 0.35% | |
| ME-522-i451 | | | 0.65% | |
| Any Unspecified Impurity | NMT 1.0 area % | | 0.08% | |
| Total Impurities | NMT 5.0 area % | | 1.5% | |
| Residual Solvents by GC | 2-Methyl- | NMT 5000 ppm | <169 | ppm |
| | tetrahydrofuran | | | |
| | Acetone | NMT 5000 ppm | 3888 | ppm |
| | Dichloromethane | NMT 600 ppm | <260 | ppm |
| | N,N- | NMT 880 ppm | <184 | ppm |
| | Dimethylformamide | | | |
| | Heptane | NMT 5000 ppm | <134 | ppm |
| | Isopropyl Acetate | NMT 5000 ppm | <171 | ppm |
| | Methanol | NMT 3000 | TBD | |
| | Pyridine | NMT 200 ppm | <195 | ppm |
| | Triethylamine | NMT 5000 ppm | <143 | ppm |
| Residual Solvents by Ion | Acetic Acid | NMT 5000 ppm | 730 | ppm |
| Chromatography | | | | |
| Elemental Impurities by | As | NMT 15 ppm | <0.01 | ppm |
| ICP-MS | Cd | NMT 5 ppm | <0.006 | ppm |
| | Hg | NMT 30 ppm | <0.08 | ppm |
| | Pb | NMT 5 ppm | <0.03 | ppm |
| | Co | NMT 50 ppm | <0.0755 | ppm |
| | V | NMT 100 ppm | <0.08 | ppm |
| | Ni | NMT 100 ppm | 8.75 | ppm |

| | | | |
|---|---|---|---|
| Water Content by Karl Fischer | Report Results | 0.56 | w/w % |
| Titration | | | |
| Malonate Content by Ion | Report Results | 17 | w/w % |
| Chromatography | | | |

| | | |
|---|---|---|
| Solid State Form by XRPD | Conforms to Reference Standard | Conforms |
| Residue on Ignition. USP | Report Results | 0.17% |
| <281> | | |
| Particle Size Analysis, USP | Report Results | X₁₀ = 1.71 μm |
| <429> | | X₅₀ = 6.89 μm |
| (X₁₀, X₅₀, X₉₀) | | X₉₀ = 25.74 μm |

Abbreviations:
NLT = Not less than;
NMT = Not more than;
NMR = Nuclear magnetic resonance;
HPLC = High performance liquid chromatography;
RRT = Relative retention time;
GC = Gas chromatography;
ppm = parts per million;
ICP-MS = Inductively Coupled Plasma-Mass Spectrometry;
XRPD = X-ray powder diffraction;
X = Particle size distribution;
TBD = Not measured

### Example 10: Manufacturing of Voruciclib Malonate Tablets

The batch formulas used in the manufacture of voruciclib malonate tablets, 50 mg and 100 mg, are provided in Table 11. The engineering batch sizes ranged from 1.2-1.3 kg, but clinical batch sizes will vary based on need.

TABLE 11
Batch Formula for Voruciclib Malonate Tablets, 50 mg and 100 mg

| | 50 mg | 100 mg | | |
|---|---|---|---|---|
| | Tablets | Tablets | | |
| | Amount (g) | Amount (g) | | Quality |
| Component | per Batch | per Batch | % w/w | Standard |
| Voruciclib Malonate^{a} | 261.72 | 283.53 | 21.81 | In-house |
| Microcrystalline | 171.84 | 186.16 | 14.32 | USP/NF |
| Cellulose (PH102) | | | | |
| Lactose Monohydrate | 102.6 | 111.15 | 8.55 | USP/NF |
| Dibasic Calcium | 519.84 | 563.16 | 43.32 | USP/NF |
| Phosphate, Dihydrate | | | | |
| Sodium Bicarbonate | 48 | 52 | 4.00 | USP/NF |
| Sodium Starch | 72 | 78 | 6.00 | USP/NF |
| Glycolate | | | | |
| Colloidal Silicon | 12 | 13 | 1.00 | USP/NF |
| Dioxide | | | | |
| Magnesium Stearate | 12 | 13 | 1.00 | USP/NF |
| TOTAL | 1,200 | 1,300 | 100.0 | N/A |
| Opadry II 85F | 66^{b} | 66^{b} | 3.0^{c} | Proprietary^{d} |
| 18422 White | | | | |
| Purified Water | 374 | 374 | NA | USP |

Abbreviations:
N/A = Not applicable.
^{a}Exact amount is corrected for drug substance assay (free base on the anhydrous, solvent free basis). Salt conversion factor is 0.8187 so 261.72 g of voruciclib malonate equals 214.3 g of voruciclib free base and 283.53 g of voruciclib malonate equals 232.1 g of voruciclib free base. Any adjustment to voruciclib malonate is accompanied with an adjustment to Lactose Monohydrate to maintain the target core tablet weight.
^{b}Amounts based on a 3% target weight gain for a tablet batch size of 1,100 grams, includes ~2-fold overage for processing.
^{c}Film coat target weight gain 3%, water removed during processing.
^{d}The film-coat applied to the tablets is a non-compendial, proprietary excipient provided by Colorcon and all components are compendial grade.

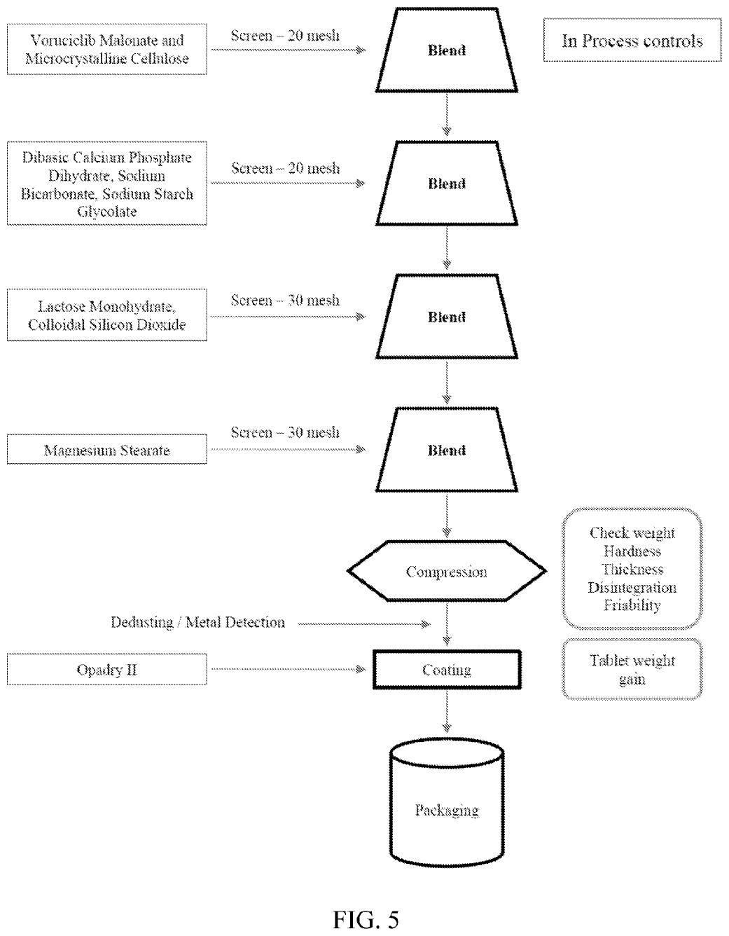

Voruciclib malonate and all excipients (microcrystalline cellulose, lactose monohydrate, dibasic calcium phosphate dihydrate, sodium bicarbonate, sodium starch glycolate, colloidal silicon dioxide, and magnesium stearate) are screened prior to use (FIG. 5).

A blend is made using microcrystalline cellulose and the voruciclib malonate. Screened dibasic calcium phosphate dihydrate, sodium bicarbonate, and sodium starch glycolate are added to the initial blend and blended. Screened lactose monohydrate and colloidal silicon dioxide are screened, added to the blend, and blended. Magnesium stearate is screened, added to the blend, and mixed. The final blend is tableted using a suitable tableting press. The core tablets are dedusted and passed through a metal detector prior to being coated with Opadry II 85F18422 White in a suitable pan coater. The coated tablets are inspected before being bulk packaged into a LDPE (Low Density Polyethylene) polybag and sealed. This polybag is placed inside a second polybag along with two 100-gram desiccant bags and sealed. The double polybagged tablets are than placed in a hard sided shipping container. The bulk packed tablets are shipped to the packaging site where they are packaged into high density polyethylene bottles and polypropylene closures with a desiccant.

In-process controls and observations for voruciclib malonate tablet manufacturing are presented in Table 12.

TABLE 12
In-Process Controls for Voruciclib
Malonate Tablets, 50 mg and 100 mg

| Process | Testing Parameter | Acceptance Criteria |
|---|---|---|
| Tableting | Individual Tablet weight | Within ± 5% of target |
| | | weight |
| Tableting | Average table weight | Within ± 3.0% of target |
| | (Based on 10 Tablets) | weight |
| Tableting | Average Hardness (10 Tablets) | Report Results |
| Tableting | Average Thickness (10 Tablets) | Report Results |
| Tableting | Disintegration (6 Tablets) | NMT 3 Minutes |
| Tableting | Friability | NMT than 1.0% Loss |
| Coating | Tablet Weight Gain (10 Tablets) | Average Tablet Weight + |
| | | 2-4% (Target 3%) |

NMT = Not more than

### Example 11: Dissolution of Voruciclib Malonate Tablets

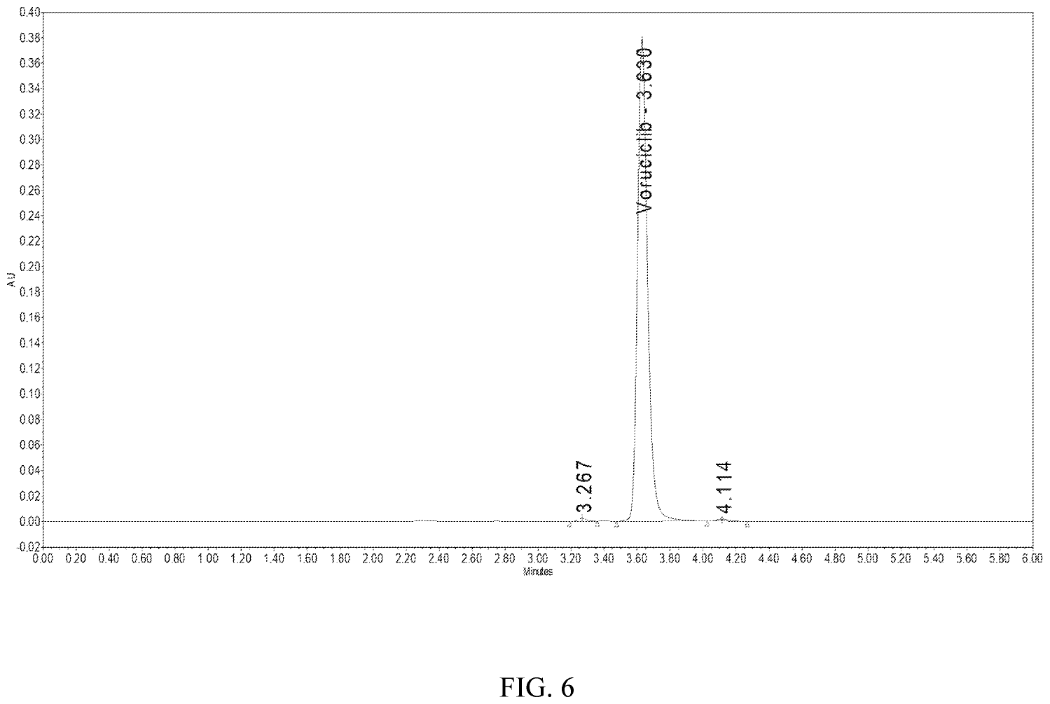

Dissolution tests were conducted according to USP <711> using Apparatus 2 (paddles with sinkers) at 50 rpm. Sinkers were employed due to the inclusion of sodium bicarbonate in the formulation, which may cause the tablets to float. The dissolution media was 900 mL of 0.1 N HCl with detection by the isocratic HPLC method described in Table 13. The sample timepoint is 30 minutes. An example chromatogram is shown in FIG. 6.

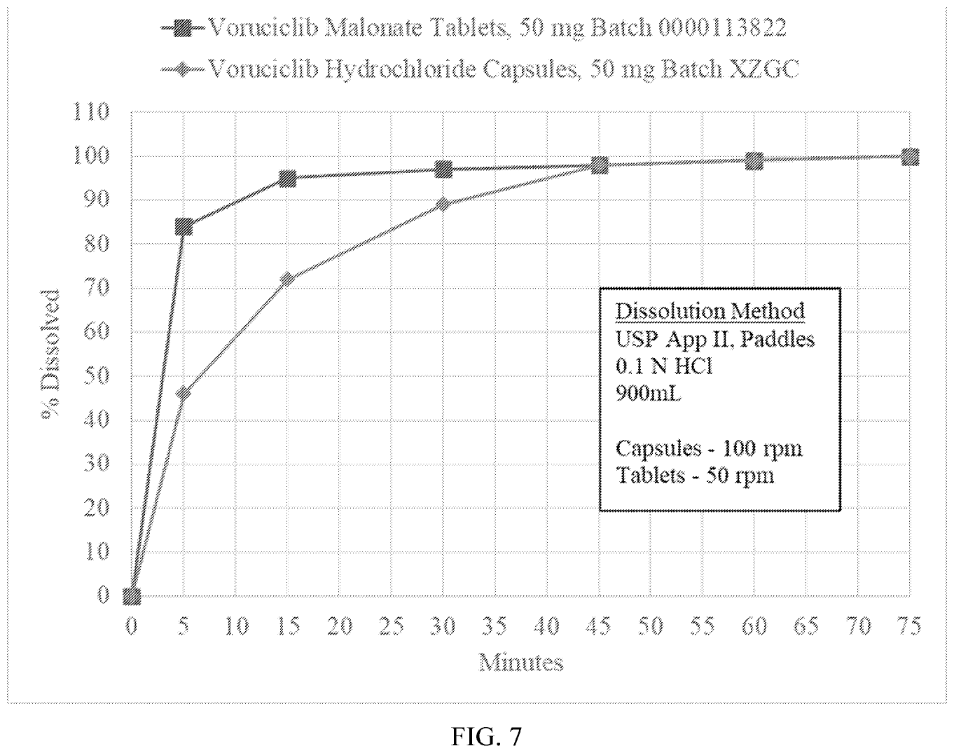

The dissolution profile of voruciclib malonate tablets (50 mg, Batch 000011382) was also compared to that of voruciclib hydrochloride capsules (50 mg, Batch XZGC) (Table 14 and FIG. 7)

TABLE 13
Reverse Phase HPLC Method Used for Dissolution Testing
of Voruciclib Malonate Tablets, 50 and 100 mg

| HPLC | |
|---|---|
| Component | Requirement/Parameter |
| Column | YMC Pack ODS A (250 mm × 4.6 mm, 5 μm |
| | particle size) |
| Mobile Phase | A: 0.05% TFA in water |
| | B: Acetonitrile/isopropanol/TFA (900:100:0.5 mL) |
| | Isocratic elution of 42% (A):58% (B) |
| Detection | UV at 264 nm |
| Column | 30° C. |
| Temperature | |
| Injection Volume | 10 μL |
| Flow Rate | 1.0 mL/min |
| Voruciclib | Approximately 3.8 minutes |
| Elution Time | |

Abbreviations:
TFA = Trifluoroacetic acid;
UV = Ultraviolet spectroscopy

TABLE 14
Dissolution time of voruciclib malonate tablets
and voruciclib hydrochloride capsules

| Sampling | Voruciclib Hydrochloride | Voruciclib Malonate |
|---|---|---|
| Time | Capsules, 50 mg | Tablets, 50 mg |
| (Mins) | Batch XZGC | Batch 0000113822 |
| 0 | 0 | 0 |
| 5 | 46 | 84 |
| 15 | 72 | 95 |
| 30 | 89 | 97 |
| 45 | 98 | 98 |
| 60 | 99 | 99 |
| 75 | 100 | 100 |

### Example 12: Stability of Voruciclib Malonate Tablets

Stability data was generated for the development Batch 108700C1 (Tables 15-17). The formulation differs from the intended clinical formulation in that it does not contain colloidal silicon dioxide. The manufacturing process for this batch was similar to that described in Example 10, with the exception that colloidal silicon dioxide was not added during the process. Stability study samples were packaged in the containers described in Example 9.

TABLE 15
Stability Data for Voruciclib Malonate Tablets, 50 mg Development
Batch 108700C1, Stored at 25° C./60% RH, in HDPE Bottles

| | Acceptance | Time Points (Months) | | |
|---|---|---|---|---|
| Attribute | Criteria | Initial | 1 | 3 |
| Appearance by Visual | Report Results | Off white | Off white | Off white |
| Inspection | | oval shaped | oval shaped | oval shaped |
| | | tablet | tablet | tablet |
| Assay by HPLC | 90.0-110.0% | 98.9 | 102.0 | 99.8 |
| | of Label Claim | | | |
| Organic Impurities | | | | |
| by HPLC | | | | |
| ME-522-i455 | NMT 1.0% area | 0.34 | 0.33 | 0.34 |
| ME-522-i451 | NMT 1.0% area | 0.66 | 0.64 | 0.65 |
| Any Unspecified | NMT 0.5% area | 0.11 | 0.09 | 0.10 |
| Degradation Product^{a} | | | | |
| Total Degradation | NMT 5.0% area | 1.3 | 1.1 | 1.3 |
| Products^{b} | | | | |
| Water Content. USP <921> | Report Results, | 2.2 | 2.1 | 1.8 |
| | % w/w | | | |
| Dissolution, USP <711> | | | | |
| Mean | Report Results | 99 | 97 | 98 |
| Range | (% Dissolved at | 95-104 | 94-99 | 95-100 |
| | 30 mins) | | | |

Abbreviations:
NMT = Not More Than;
HPLC = High performance liquid chromatography;
RH = Relative humidity
^{a} Largest Unspecified Degradation Product >0.05% area. Peaks designated as Synthetic Impurities at the time of analysis are excluded
^{b}Sum of degradation products ≥0.05% area

TABLE 16
Stability Data for Voruciclib Malonate Tablets, 50 mg Development
Batch 108700C1, Stored at 40° C./75% RH, in HDPE Bottles

| | Acceptance | Time Points (Months) | | |
|---|---|---|---|---|
| Attribute | Criteria | Initial | 1 | 3 |
| Appearance by Visual | Report Results | Off white | Off white | Off white |
| Inspection | | oval shaped | oval shaped | oval shaped |
| | | tablet | tablet | tablet |
| Assay by HPLC | 90.0-110.0% | 98.9 | 100.7 | 101.6 |
| | of Label Claim | | | |
| Organic Impurities | | | | |
| by HPLC | | | | |
| ME-522-i455 | NMT 1.0% area | 0.34 | 0.33 | 0.34 |
| ME-522-i451 | NMT 1.0% area | 0.66 | 0.64 | 0.65 |
| Any Unspecified | NMT 0.5% area | 0.11 | 0.09 | 0.08 |
| Degradation Product^{a} | | | | |
| Total Degradation | NMT 5.0% area | 1.3 | 1.1 | 1.2 |
| Products^{b} | | | | |
| Water Content. USP <921> | Report Results, | 2.2 | 1.9 | 2.4 |
| | % w/w | | | |
| Dissolution, USP <711> | | | | |
| Mean | Report Results | 99 | 99 | 99 |
| Range | (% Dissolved at | 95-104 | 96-103 | 98-100 |
| | 30 Mins) | | | |

Abbreviations:
NMT = Not More Than;
HPLC = High performance liquid chromatography;
RH = Relative humidity
^{a}Largest Unspecified Degradation Product >0.05% area. Peaks designated as Synthetic Impurities at the time of analysis are excluded
^{b}Sum of degradation products ≥0.05% area

TABLE 17
Stability Data for Voruciclib Malonate Tablets, 50 mg Development
Batch 108700C1 Stored at 50° C./Ambient RH, in HDPE Bottles

| | | Time Points (Months) | | |
|---|---|---|---|---|
| Attribute | Acceptance Criteria | Initial | 1 | 2 |
| Appearance by Visual | Report Results | Off white | Off white | Off white |
| Inspection | | oval shaped | oval shaped | oval shaped |
| | | tablet | tablet | tablet |
| Assay by HPLC | 90.0-110.0% of Label Claim | 98.9 | 101.4 | 98.2 |
| Organic Impurities by HPLC | | | | |
| ME-522-i455 | NMT 1.0% area | 0.34 | 0.33 | 0.34 |
| ME-522-i451 | NMT 1.0% area | 0.66 | 0.64 | 0.66 |
| Any Unspecified | NMT 0.5% area | 0.11 | 0.11 | 0.12 |
| Degradation Product | | | | |
| Total Degradation Products | NMT 5.0% area | 1.3 | 1.2 | 1.2 |
| Water Content. USP <921> | Report Results, % w/w | 2.2 | 1.8 | 1.8 |
| Dissolution, USP <711> | | | | |
| Mean | Report Results | 99 | 101 | 96 |
| Range | (% Dissolved at 30 Mins) | 95-104 | 97-104 | 95-98 |

Abbreviations:
NMT = Not More Than;
HPLC = High performance liquid chromatography;
RH = Relative humidity
^{a} Largest Unspecified Degradation Product >0.05% area. Peaks designated as Synthetic Impurities at the time of analysis are excluded
^{b} Sum of degradation products ≥0.05% area

The stability data for voruciclib malonate tablets, 50 mg development batch 108700C1 show that there was no significant change in any of the attributes tested through three months at storage conditions of 25° C./60% relative humidity (RH) or at 40° C./75% RH and through two months storage at 50° C./Ambient RH.

Stability data was generated for the development Batch 02MEI06-01-107 (Tables 18 and 19). The formulation contains colloidal silicon dioxide. The manufacturing process for this batch was similar to that described in Example 10. Stability study samples were packaged in the containers described in Example 9.

TABLE 18
Stability Data for Voruciclib Malonate Batch 02MEI06-01-107 at 25° C./60% RH

| | Acceptance | | | | |
|---|---|---|---|---|---|
| Attribute | Criteria^{a} | Initial | 1 months | 3 months | 6 months |
| Appearance by | Report Results | Pale | Light | Light | Light |
| visual inspection | | yellow solid | yellow solid | yellow solid | yellow solid |
| Assay by HPLC | 95.0-105.0% | 96.9 | 96.8 | 97.3 | 97.7 |
| (% w/w, as is) | | | | | |
| Purity by HPLC (% area) | NLT 95.0% | 98.5 | 98.6 | 98.7 | 98.7 |
| Organic Impurities by HPLC | | % Area | | | |
| (% area) | | | | | |
| RRT 0.83 | Report Results | 0.06 | 0.06 | 0.06 | 0.05 |
| RRT 0.97 | | 0.55 | 0.55 | 0.56 | 0.52 |
| RRT 0.99 | | 0.62 | 0.59 | 0.59 | 0.58 |
| RRT 1.05 | | 0.15 | 0.09 | 0.06 | 0.05 |
| RRT 1.06 | | 0.11 | 0.08 | 0.08 | 0.08 |
| Total Impurities (% area) | NMT 5.0% | 1.53 | 1.38 | 1.36 | 1.32 |
| Water Content by KF (% w/w) | Report Results | 0.56 | 0.47 | 0.18 | 0.36 |
| XRPD | Report Results | Crystalline | Crystalline | Crystalline | Crystalline |

Abbreviations:
HPLC = High performance liquid chromatography;
KF = Karl Fischer;
NLT = Not less than;
XRPD = X-ray powder diffraction;
RRT = Relative retention time;
RH = Relative humidity
^{a}The acceptance criteria listed are those in place when the lot was tested at release.

TABLE 19
Stability Data for Voruciclib Malonate Batch 02MEI06-01-107 at 40° C./75% RH

| | Acceptance | | | | |
|---|---|---|---|---|---|
| Attribute | Criteria^{a} | Initial | 1 months | 3 months | 6 months |
| Appearance | Report Results | Pale | Light | Light | Light |
| | | yellow solid | yellow solid | yellow solid | yellow solid |
| Assay by HPLC | 95.0-105.0% | 96.9 | 96.4 | 97.3 | 97.5 |
| (% w/w. as is) | | | | | |
| Purity by HPLC (% area) | NLT 95.0% | 98.5 | 98.7 | 98.7 | 98.8 |
| Organic Impurities by HPLC | Report Results | % Area | | | |
| RRT 0.83 | | 0.06 | 0.06 | 0.06 | 0.05 |
| RRT 0.97 | | 0.55 | 0.56 | 0.56 | 0.52 |
| RRT 0.99 | | 0.62 | 0.58 | 0.59 | 0.58 |
| RRT 1.05 | | 0.15 | 0.06 | — | — |
| RRT 1.06 | | 0.11 | 0.08 | 0.08 | 0.08 |
| Total Impurities (% area) | NMT 5.0% | 1.53 | 1.33 | 1.29 | 1.24 |
| Water Content by KF (% w/w) | Report Results | 0.56 | 0.44 | 0.14 | 0.46 |
| XRPD | Report Results | Crystalline | Crystalline | Crystalline | Crystalline |

Abbreviations:
HPLC = High performance liquid chromatography;
KF = Karl Fischer;
NLT = Not less than;
XRPD = X-ray powder diffraction;
RRT = Relative retention time;
RH = Relative humidity;
“—” = below reporting threshold.
^{a}The acceptance criteria listed are those in place when the lot was tested at release.

Stability data obtained for voruciclib malonate, Batch 02MEI06-01-107 is presented Tables 18 and 19. No significant changes were been observed after storage for 6-months at long-term conditions (25° C./60% relative humidity (RH)) or for 6-months at accelerated conditions (40° C./75% RH).

The stability protocol for voruciclib malonate tablets, 50 mg development Batch 108700C1, is shown in Table 20. This batch was manufactured at laboratory and differs from the intended clinical formulation in that it does not contain colloidal silicon dioxide. The manufacturing process for this batch was similar to that described in Example 10, with the exception that colloidal silicon dioxide was not added during the process. The batch was tested by the analytical procedures described herein. Stability study samples were packaged in the containers described in Example 9, with the exception that there were 7 tablets per bottle rather than 14.

TABLE 20
Test Parameters for Stability Study for Voruciclib
Malonate Tablets, 50 mg Development Batch 108700C1

| | Time Points (months) | | | | | | | | | |
|---|---|---|---|---|---|---|---|---|---|---|
| Storage Condition | 0 | 1 | 2 | 3 | (6) | (9) | (12) | (18) | (24) | (36) |
| 25° C./60% RH | X | X | — | X | X | X | X | X | X | X |
| 40° C./75% RH | | X | — | X | X | — | — | — | — | — |
| 50° C./Ambient RH | | X | X | — | — | — | — | — | — | — |

( ) = Optional Time points
X = Appearance, Assay and Organic Impurities, Water Content, and Dissolution
RH = Relative humidity

The stability protocol for voruciclib malonate tablets, 50 mg and 100 mg, engineering batches 0000113822 and 0000113823, manufactured at a laboratory scale is shown in Table 21. These batches were manufactured according to the process described in Example 10. The batch was tested by the analytical procedures described herein. Stability study samples were packaged in the containers described in Example 9, with the exception that there were 7 tablets per bottle rather than 14.

TABLE 21
Test Parameters for Stability Studies for Voruciclib
Malonate Tablets, 50 mg and 100 mg, Engineering
Batches 0000113822 and 0000113823

| | Time Points (months) | | | | | | | | |
|---|---|---|---|---|---|---|---|---|---|
| Storage Condition | 0 | 1 | 3 | 6 | 9 | 12 | 18 | 24 | 36 |
| 25° C./60% RH | R | X | X | X | X | X | X | X | X |
| 30° C./65% RH | | — | C | C | C | C | — | — | — |
| 40° C./75% RH | | X | X | X | — | — | — | — | — |

R = Release tests
X = Appearance, Assay and Organic Impurities, Water Content, and Dissolution
C = Contingency. Samples are stored, but tested only on failure of accelerated testing
— = Not Scheduled
RH = Relative humidity

The first batches of voruciclib malonate tablets, 50 mg and 100 mg, intended for use in clinical trials, were manufactured according to the process described Example 10 and will be placed on stability per the protocol shown in Table 22. Stability study samples will be packaged in containers (14-count) as described in Example 9.

TABLE 22
Test Parameters for Stability Studies for Voruciclib
Malonate Tablets, 50 mg and 100 mg, Clinical Batches

| | Time Points (months) | | | | | | | | |
|---|---|---|---|---|---|---|---|---|---|
| Storage Condition | 0 | 1 | 3 | 6 | 9 | 12 | 18 | 24 | 36 |
| 25° C./60% RH | R | X | X | X | X | X, M | X | X, M | X, M |
| 30° C./65% RH | | — | C | C | C | C | — | — | — |
| 40° C./75% RH | | X | X | X | — | — | — | — | — |

R = Release tests
M = Microbial limits and specified organism
X = Appearance, Assay and Organic Impurities, Water Content, and Dissolution
C = Contingency. Samples are stored, but tested only on failure of accelerated testing
— = Not Scheduled
RH = Relative humidity

The stability data for voruciclib malonate tablets, 50 mg development batch 108700C1, presented in Tables 15-17 show that there was no significant change in any of the attributes tested through three months at storage conditions of 25° C./60% relative humidity (RH) or at 40° C./75% RH and through two months storage at 50° C./Ambient RH.

### Example 13. Properties of Voruciclib Malonate

Voruciclib malonate's chemical (IUPAC) name is (2R,3S)-3-(2-(2-chloro-4-(trifluoromethyl)phenyl)-5,7-dihydroxy-4-oxo-4H-chromen-8-yl)-2-(hydroxymethyl)-1-methylpyrrolidin-1-ium-2-carboxyacetate. Common synonyms used for voruciclib malonate are (2R,3S)-3-(2-(2-chloro-4-(trifluoromethyl)phenyl)-5,7-dihydroxy-4-oxo-4H-chromen-8-yl)-2-(hydroxymethyl)-1-methylpyrrolidin-1-ium malonate and 2-(2-chloro-4-(trifluoromethyl)phenyl)-5,7-dihydroxy-8-((2R,3S)-2-(hydroxymethyl)-1-methylpyrrolidin-3-yl)-4H-chromen-4-one malonic acid salt. Table 23 provides some general properties of voruciclib malonate.

TABLE 23
General Properties of Voruciclib Malonate

| Physical/Chemical | |
|---|---|
| Properties | Description |
| Physical Description | Yellow to Greenish-yellow solid |
| Melting Point (DSC) | 183.8° C. (185.1° C., decomposition) |
| Optical Rotation | +0.064° in DMSO and +0.048° in MeOH |
| pKa | 6.46, 9.59, and 12.13 |
| Hygroscopicity | Non-hygroscopic |
| Solid State | The current manufacturing process consistently |
| Polymorphic Form | produces voruciclib malonate drug substance |
| | with a single polymorph, designated as Form 1, |
| | which is an anhydrous, non-solvated crystalline |
| | material. |

Abbreviations:
DMSO = Dimethyl sulfoxide; MeOH = Methanol; DSC = Differential scanning calorimetry.

TABLE 24
pH Range Solubility of Voruciclib Malonate in Biorelevant Media

| pH | | Solubility |
|---|---|---|
| (Medium) | Simulated Situation | (mg/mL) |
| pH 1.6 | Stomach after glass of water | >13.37/11.05^{a} |
| (FaSSGF) | | |
| pH 3.0 | Gastric fluid after high-fat meal, late | 11.28 |
| (FEDGAS) | stage of stomach emptying after dosage | |
| pH 4.5 | Gastric fluid after high-fat meal, mid | 4.35 |
| (FEDGAS) | stage of stomach emptying after dosage | |
| pH 5.0 | Upper intestine after a meal | <0.004 |
| (FeSSIF) | | |
| pH 6.0 | Gastric fluid after high-fat meal, early | 2.88 |
| (FEDGAS) | stage of stomach emptying after dosage | |
| pH 6.5 | Upper intestine after a glass of water | 0.23 |
| (FaSSIF) | | |

Abbreviations:
FaSSGF = Fasted State Simulated Gastric Fluid; FEDGAS = Fed State Simulated Gastric Fluid; FeSSIF = Fed State Simulated Intestinal Fluid; FaSSIF = Fasted State Simulated Intestinal Fluid.
^{a}Sample was tested in duplicate and both values are reported.

Voruciclib exhibits solubility and gelling phenomena dependent on factors including concentration (of suspension used for solubility measurement), pH, and buffer system. In general, voruciclib was more prone to gel at suspension concentrations >5 mg/mL and solubilities >0.5 mg/mL. Gelling was observed at pH 1.6 (FaSSGF) and pH 6.5 (FaSSIF) (Table 24).

### X-ray Crystallography

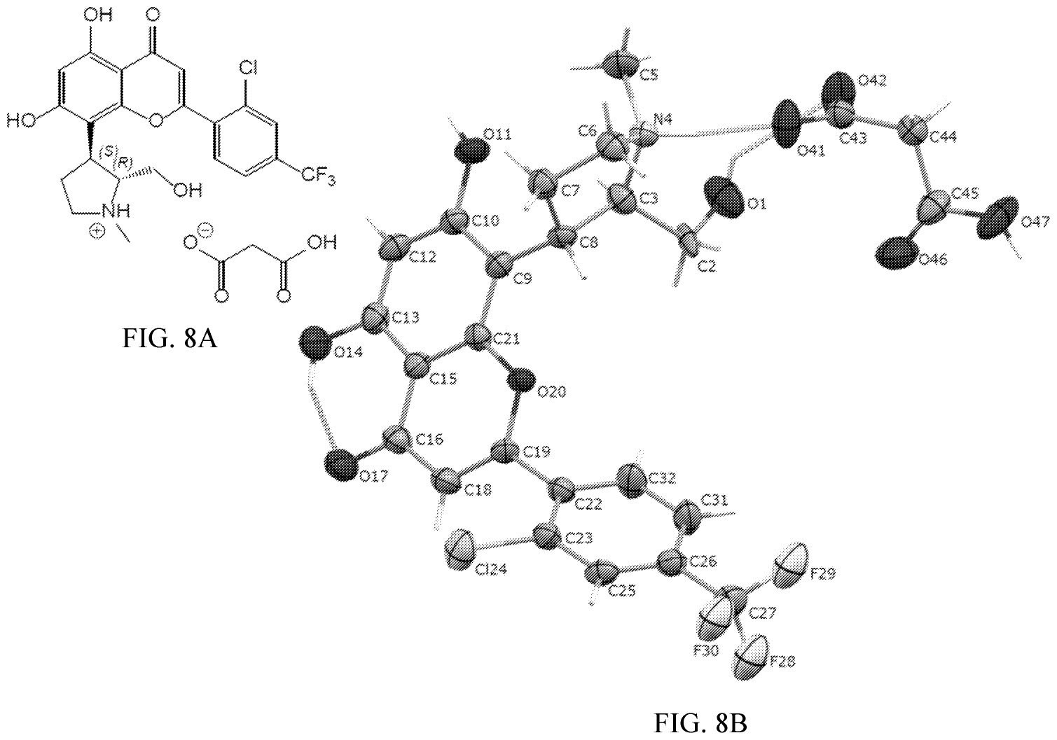

The single crystal measurements were performed on a Nonius Kappa-CCD instrument. The data were collected at 296 K. The full sphere data were collected up to θ=27.5° resulting in 20755 reflections (5650 independent). Data reduction was performed using HKL Scalepak and cell parameters were obtained using Denzo and Scalepak from 26946 reflections within 0 range 1 to 27.5°. The structure was solved using direct methods by SHELXT-2014/7. The structure was refined by least square full matrix refinement using SHELXL-2014/7. During the refinement it became apparent that the trifluoromethyl as well as the alcohol group of the voruciclib malonate cation were disordered into two positions. The applied model for disorder was refined anisotropically with the same thermal parameters for all atoms in the disordered groups. All of the H atoms were implemented from the geometry of the molecule (C and N connected) or based on the geometry of the hydrogen bonds. During the refinement, all H atoms were kept with 150% or 120% thermal parameter of the neighboring non-H atoms. The absolute configuration was established using Flack parameter, which had a value of x=0.06±0.05 and was determined using 1377 quotients [(I+)−(I−)]/[(I+)+(I−)]. The X-ray crystal structure confirms the chemical structure of voruciclib malonate (FIG. 8B).

Voruciclib malonate (Form 1) crystallizes in the monoclinic centrosymmetrical space group P21, with one molecule of active pharmaceutical ingredient (API) and one molecule of malonic acid in the asymmetric unit (Table 22). The stereocenters at C3 and C8 are in the R and s configuration, respectively. The data confirm the expected chemical structure shown in FIGS. 8A-8B. The crystallographic data showing the final refinement parameters are reported in Table 25.

TABLE 25
Crystallographic Data for Voruciclib (ME-522) Malonate (Form 1)

| Parameters | Data | |
|---|---|---|
| Compound Name | ME-522 Malonate | |
| Form | Form 1 | |
| Empirical formula | C₂₂H₂₀ClF₃NO₂⁺ C₃H₃O₄⁻ | |
| Formula weight | 573.90 | |
| T [K] | 296 | (2) |

| | |
|---|---|
| λ [Å] | 0.71073 |
| Crystal system | Monoclinic |
| Space group | P2₁ |

Unit cell parameters

| a [Å] | 12.1252 | (6) |
|---|---|---|
| b [Å] | 7.3672 | (3) |
| c [Å] | 13.9454 | (6) |
| β [°] | 94.2416 | (18) |
| V [Å³] | 1242.31 | (10) |
| Z | 2 | |
| Dₑₐₖ [g/cm³] | 1.534 | |
| μ [mm⁻³] | 0.233 | |
| F(000) | 592 | |
| Crystal size [mm³] | 0.450 × 0.150 × 0.150 | |
| θ range for data collection [°] | 2.9 to 32.6. | |
| Reflections collected | 58124 | |
| Independent reflections | 9027 [R_{ind} = 0.0697] | |
| Completeness to θ = 25.242° [%] | 99.6 | |
| Absorption correction | Semi-empirical from equivalents | |
| Max. and min. transmission | 0.996 and 0.804 | |
| Data/restraints/parameters | 9027/9/396 | |
| Goodness-of-fit on F² | 1.050 | |
| Final R indices [I > 2σ(I)] | R1 = 0.0533, wR2 = 0.1463 | |
| R indices (all data) | R1 = 0.0609, wR2 = 0.1531 | |
| Absolute structure parameter | 0.02 | (2) |

| | |
|---|---|
| Absolute configuration | R(C3), S(C8) |
| Largest diff. peak and hole [e/Å³] | 0.445 and −0.346 |

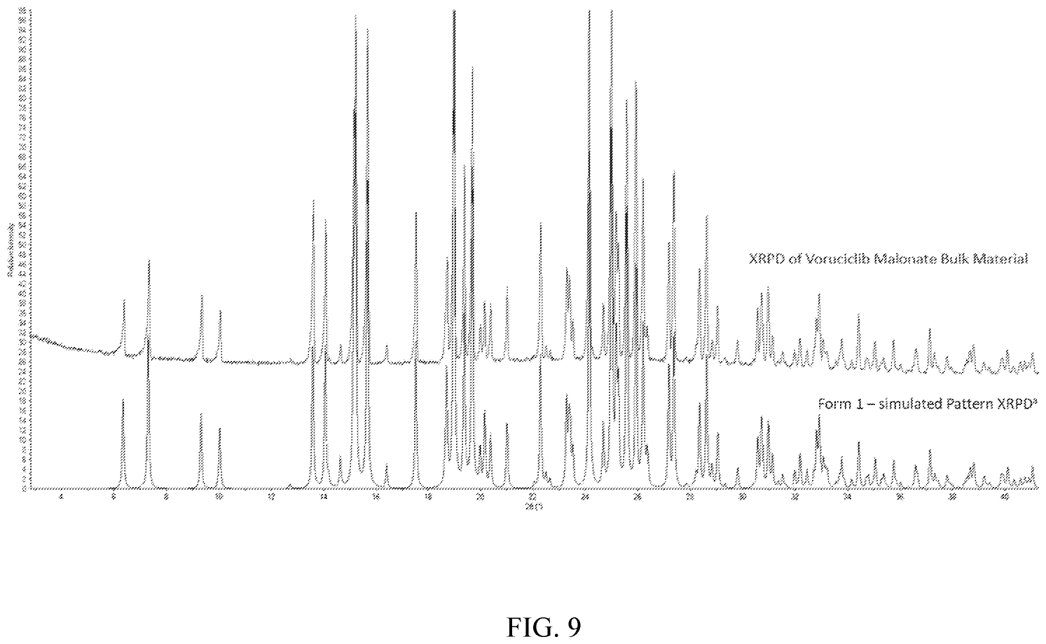

To confirm the determined single crystal structure is representative of the bulk material, the simulated powder pattern from the single crystal data was compared to the high-resolution X-ray powder diffraction (HR-XRPD) pattern obtained from the bulk material (FIG. 9). The matching XRPD patterns confirm that the obtained single crystal and the received batch (Batch 20-07211) of voruciclib are the same form (Form 1).

Amorphous voruciclib malonate was prepared by freeze-drying a free base solution containing one equimolar amount of malonic acid. The polymorph screen experiments were started with amorphous voruciclib malonate salt to favor unbiased crystallization. Form 1 was the most frequently observed form. Form 1 is anhydrous and non-hygroscopic. Three other forms were observed, but only under specific recrystallization conditions and all three novel crystalline forms appeared to be hydrates. All three forms were physically unstable and converted to Form 1 upon drying under vacuum or exposure to stress conditions (40° C./75% relative humidity).

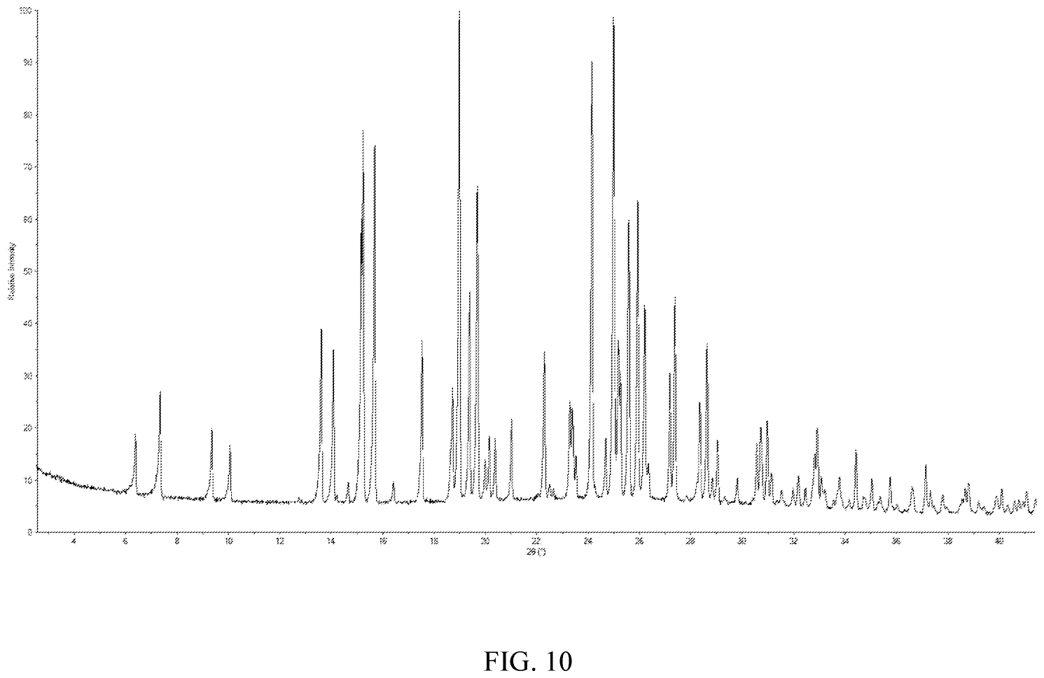

The single crystal structure of voruciclib malonate (Form 1) is monoclinic centrosymmetrical space group P21 (FIG. 8B), with one molecule of API and one molecule of malonic acid in the asymmetric unit. The XRPD diffractogram of Form 1 is presented in FIG. 10 and the characteristic peaks are listed in Table 26.

TABLE 26
XRPD Peak Data

| Peak | 2θ [°] | d [Å] | Intensity |
|---|---|---|---|
| 1 | 6.38 | 13.83 | 12 |
| 2 | 7.33 | 12.04 | 22 |
| 3 | 9.35 | 9.46 | 15 |
| 4 | 10.06 | 8.79 | 11 |
| 5 | 13.62 | 6.50 | 35 |
| 6 | 14.08 | 6.29 | 31 |
| 8 | 15.23 | 5.81 | 76 |
| 9 | 15.69 | 5.64 | 73 |
| 11 | 17.53 | 5.05 | 33 |
| 12 | 18.72 | 4.74 | 23 |
| 13 | 18.98 | 4.67 | 100 |
| 14 | 19.38 | 4.58 | 43 |
| 15 | 19.69 | 4.51 | 64 |
| 17 | 20.16 | 4.40 | 13 |
| 18 | 20.37 | 4.36 | 13 |
| 19 | 21.01 | 4.23 | 16 |
| 20 | 22.29 | 3.99 | 30 |
| 23 | 23.30 | 3.82 | 20 |
| 24 | 23.38 | 3.80 | 18 |
| 26 | 24.15 | 3.68 | 89 |
| 27 | 24.68 | 3.60 | 12 |
| 28 | 24.99 | 3.56 | 98 |
| 29 | 25.17 | 3.54 | 32 |
| 30 | 25.25 | 3.52 | 26 |

### Example 14: Synthesis of Voruciclib Malonate

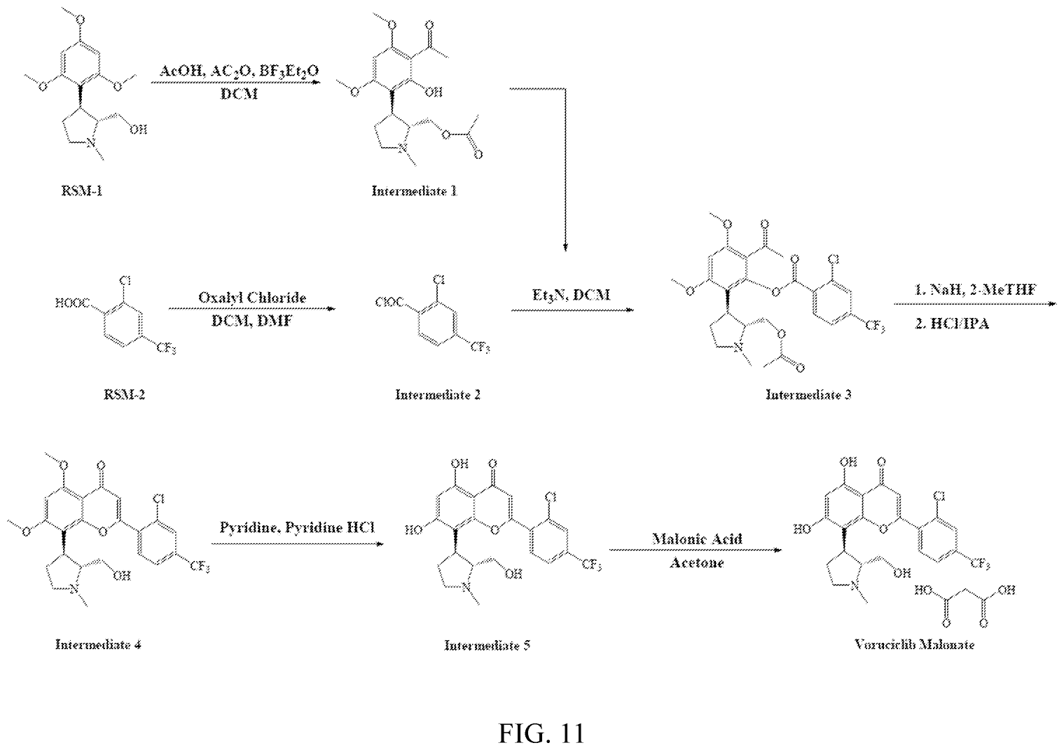

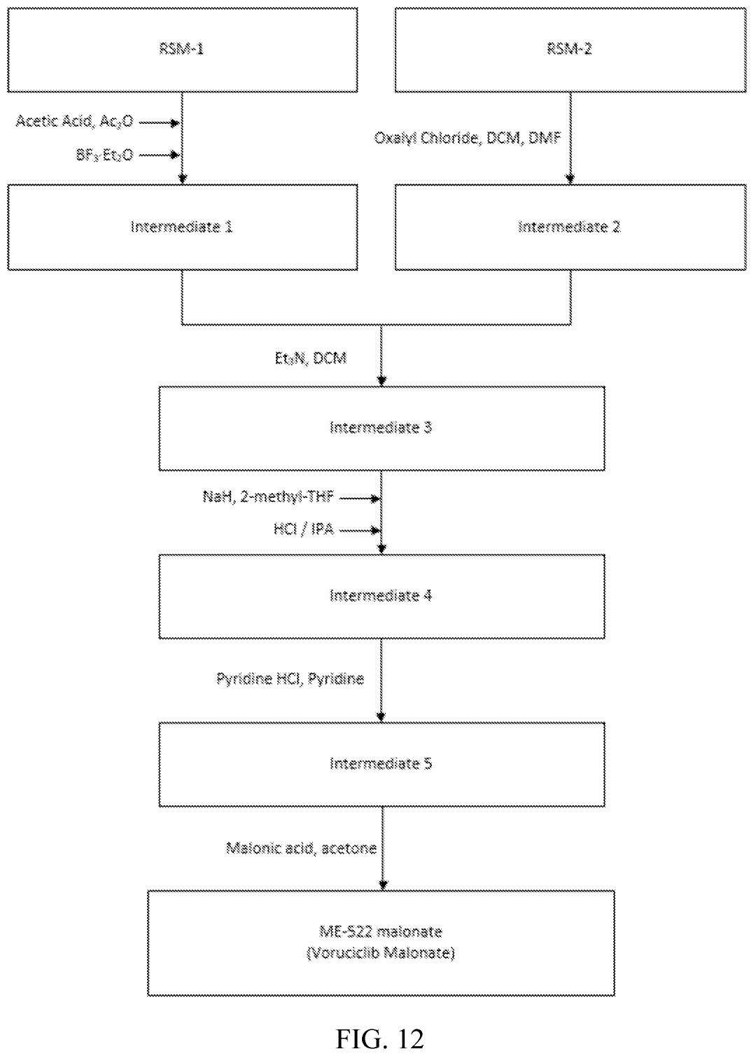

FIG. 11 provides a scheme depicting the synthesis of voruciclib malonate while FIG. 12 provides a flow diagram for the manufacturing process of voruciclib malonate.

### Synthesis of Intermediate 1

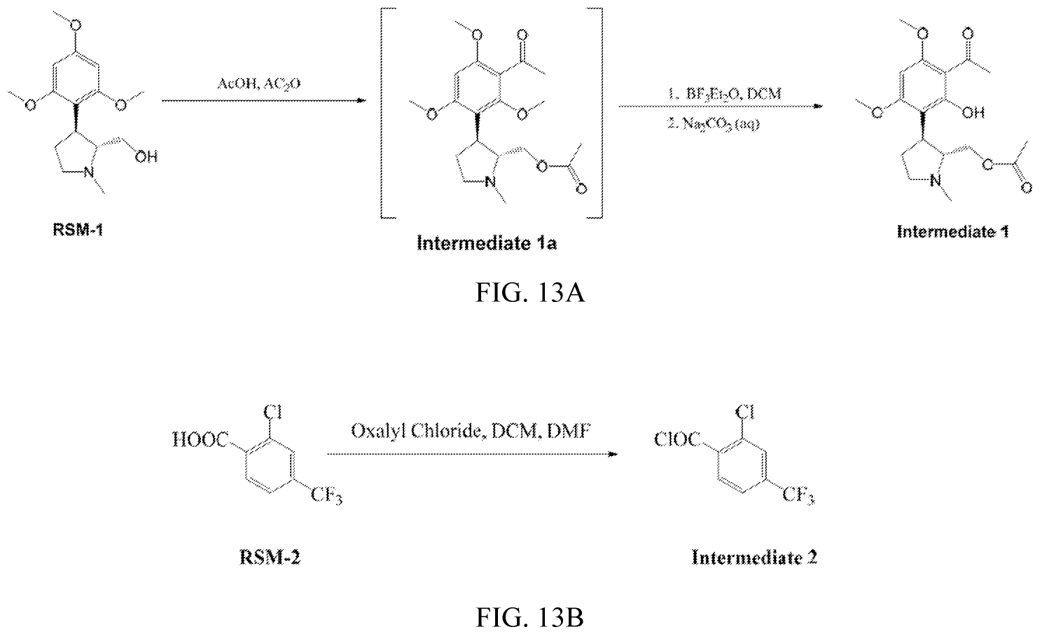

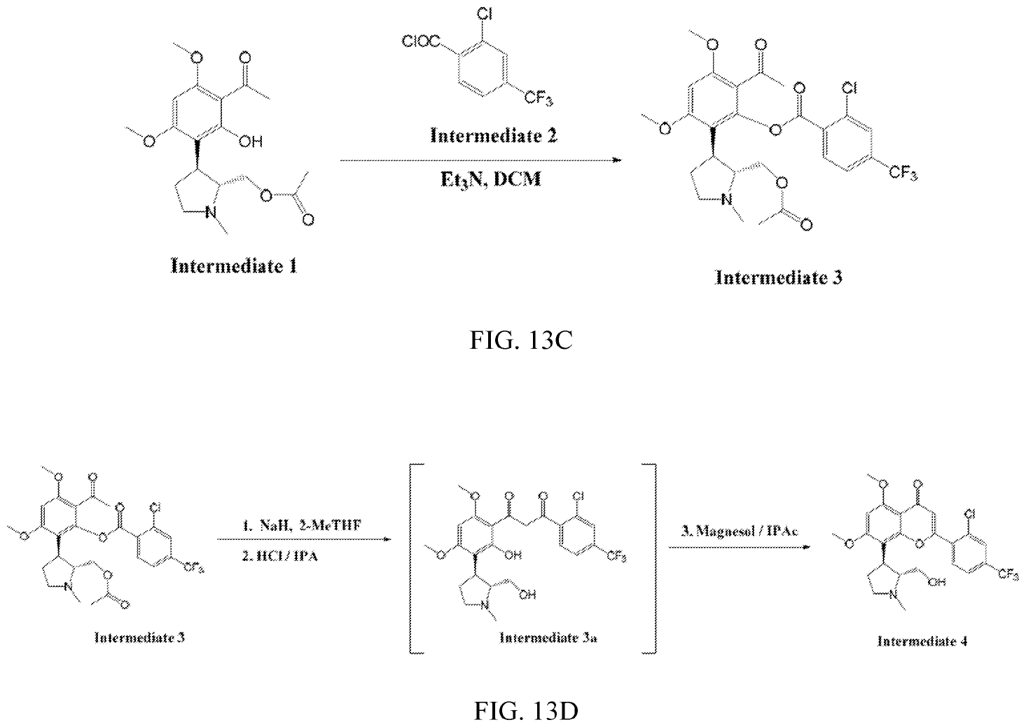

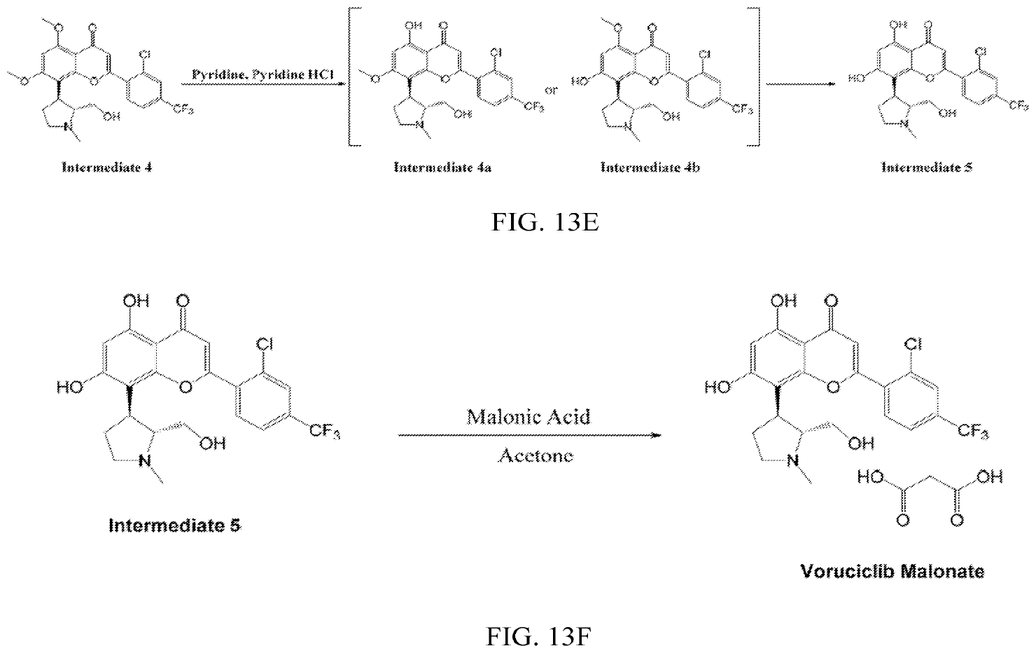

Acetic acid was charged into a reactor followed by the addition of acetic anhydride (FIG. 13A). RSM-1 was charged into the reactor and once the addition was complete agitation was continued until the reaction was deemed complete by the consumption of RSM-1. The reaction solution was treated with boron trifluoride diethyl etherate. Once the addition was complete the reactor content was heated with agitation until Intermediate 1a was consumed.

The reaction mixture was cooled, and dichloromethane was added to the reaction mixture followed by a solution of aqueous sodium carbonate. Once the addition was complete the reaction mixture was agitated at a rate to ensure adequate mixing of the biphasic mixture. Agitation was stopped to allow the phases to separate. The organic phase (bottom layer) was separated and charged into a separate container. The remaining aqueous phase was treated with dichloromethane and agitated at a rate to ensure proper mixing of the biphasic mixture. Agitation was stopped to allow the phases to separate. The organic phase (bottom layer) was separated and combined with the first organic phase. The aqueous phase was removed, and the combined organic phase was charged back into the reactor and washed twice with an aqueous sodium chloride solution. The reaction solution was concentrated via vacuum distillation. Dichloromethane was added to the reaction mixture and was used in the next step.

### Synthesis of Intermediate 2

Dichloromethane, RSM-2, and diatomaceous earth were charged into a reactor and treated with dimethylformamide (FIG. 13B). Oxalyl chloride was added to the reaction mixture and agitated. The reaction mixture was filtered, and the filtrate was concentrated by vacuum distillation. Dichloromethane was charged into the reactor containing Intermediate 2 and agitated until a homogenous solution was obtained. Intermediate 2 dissolved in dichloromethane was used in the next step.

### Synthesis of Intermediate 3

Dichloromethane and Intermediate I were charged into a reactor and agitated while the internal temperature was adjusted to 0° C. (FIG. 13C). Triethylamine was charged into the reactor followed by the addition of Intermediate 2. Once the addition was complete the reaction was agitated until Intermediate I was consumed. Water was added to the reaction solution with agitation to quench the remaining acid chloride. Agitation was stopped to allow the phases to separate. The organic phase (bottom layer) was separated and charged into a separate container.

The remaining aqueous phase was treated with dichloromethane and agitated at a rate to ensure proper mixing of the biphasic mixture. Agitation was stopped to allow the phases to separate. The organic phase (bottom layer) was separated and combined with the first organic phase. The aqueous phase was removed, and the combined organic phase was charged back into the reactor and washed with an aqueous sodium bicarbonate solution and then washed with a sodium chloride solution. The organic phase was dried with sodium sulfate, filtered, and concentrated. 2-methyltetrahydrofuran was added to Intermediate 3 and used in the next step.

### Synthesis of Intermediate 4

Sodium hydride and 2-methyltetrahydrofuran were charged into a reactor and the reaction mixture was cooled to 0° C. (FIG. 13D). Intermediate 3 was added to the reaction mixture while maintaining a consistent internal temperature of 0° C. Once the addition was complete the reactor content was heated with agitation until Intermediate 3 was consumed.

The reaction mixture was then cooled and treated with a solution of HCl in 2-propanol (IPA). The reaction mixture was concentrated via vacuum distillation and treated with hydrochloric acid. The mixture was agitated until the reaction was deemed complete and then treated with 2-methyltetrahydrofuran. The reaction mixture was treated with an aqueous solution of sodium carbonate and dichloromethane and agitated at a rate to ensure adequate mixing of the biphasic mixture.

Agitation was stopped to allow the phases to separate. The organic phase (bottom layer) was separated and charged into a separate container. The remaining aqueous phase was treated with dichloromethane and agitated to ensure proper mixing of the biphasic mixture. The aqueous phase was removed, and the combined organic phase was charged back into the reactor and washed twice with an aqueous sodium chloride solution. The organic phase was separated, dried with sodium sulfate, and filtered.

The filtrate was treated Magnesol R, filtered and concentrated via vacuum distillation. The concentrated reaction mixture was treated with isopropyl acetate to facilitate precipitation of solids, which were then collected by vacuum filtration. The wet cake was washed with isopropyl acetate/heptane solution and dried to afford Intermediate 4.

### Synthesis of Intermediate 5

Intermediate 4, pyridine hydrochloride and pyridine were charged into a reactor and heated to reflux until the reaction mixture became homogenous (FIG. 13E). The mixture was stirred until Intermediate 4. Intermediate 4a and Intermediate 4b were consumed. The reaction mixture was concentrated via atmospheric distillation and treated with methanol. The crude reaction mixture was then treated with an aqueous solution of sodium bicarbonate at which point solids precipitated from the reaction mixture. The solids were collected by vacuum filtration, washed with water and heptane, and dried to afford Intermediate 5.

### Synthesis of Voruciclib Malonate

Intermediate 5 and acetone were charged into a reactor and heated to reflux with agitation until the reaction mixture became homogenous (FIG. 13F). The reaction solution was treated with malonic acid and agitated at reflux temperature. The reaction mixture was cooled to 20° C., and held at this temperature until solids started to precipitate. After additional stirring the solids were collected by vacuum filtration and washed with acetone. The wet cake was dried under vacuum to afford voruciclib malonate.

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

As used herein, the term “about” preceding a quantity indicates a variance from the quantity. The variance may be caused by manufacturing tolerances or may be based on differences in measurement techniques. The variance may be up to 10% from the listed value in some instances. Those of ordinary skill in the art would appreciate that the variance in a particular quantity may be context dependent and thus, for example, the variance in a dimension at a micro or a nano scale may be different than variance at a meter scale.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Terms such as “top,” “bottom,” “front.” “rear” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Furthermore, to the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology.

1. A formulation comprising between about 15% to about 35% w/w voruciclib malonate and one or more pharmaceutically acceptable excipients.

2. The formulation of claim 1, comprising between about 18% to about 30% w/w voruciclib malonate.

3. The formulation of claim 1, comprising about 18%, about 19%, about 20%, about 21%, about 22%, about 23%, about 24%, about 25%, about 26%, about 27%, or about 28% w/w voruciclib malonate.

4. The formulation of claim 1, comprising between about 20% to about 23% w/w voruciclib malonate.

5. The formulation of any one of claims 1 to 4, wherein the one or more pharmaceutically acceptable excipients comprise about 5% to about 37% w/w microcrystalline cellulose.

6. The formulation of any one of claims 1 to 4, wherein the one or more pharmaceutically acceptable excipients comprise about 10%, about 11%, about 12%, about 13%, about 14%, about 15%, about 16%, about 17%, about 18%, about 19%, or about 20% w/w microcrystalline cellulose.

7. The formulation of any one of claims 1 to 6, wherein the one or more pharmaceutically acceptable excipients comprise about 1% to about 48% w/w lactose monohydrate.

8. The formulation of any one of claims 1 to 6, wherein the one or more pharmaceutically acceptable excipients comprise about 2%, about 3%, about 4%, about 5%, about 6%, about 7%, about 8%, about 9%, about 10%, about 11%, about 12%, about 13%, about 14%, or about 15% w/w lactose monohydrate.

9. The formulation of any one of claims 1 to 8, wherein the one or more pharmaceutically acceptable excipients comprise about 20% to about 70% w/w dibasic calcium phosphate dihydrate.

10. The formulation of any one of claims 1 to 8, wherein the one or more pharmaceutically acceptable excipients comprise about 34%, about 35%, about 36%, about 37%, about 38%, about 39%, about 40%, about 41%, about 42%, about 43%, about 44%, about 45%, about 46%, about 47%, about 48%, about 49%, or about 50% w/w dibasic calcium phosphate dihydrate.

11. The formulation of any one of claims 1 to 10, wherein the one or more pharmaceutically acceptable excipients comprise about 0.1% to about 15% w/w sodium bicarbonate.

12. The formulation of any one of claims 1 to 10, wherein the one or more pharmaceutically acceptable excipients comprise about 2%, about 3%, about 4%, about 5%, about 6%, about 7%, about 8%, about 9%, or about 10% w/w sodium bicarbonate.

13. The formulation of any one of claims 1 to 12, wherein the one or more pharmaceutically acceptable excipients comprise about 1% to about 20% w/w sodium starch glycolate.

14. The formulation of any one of claims 1 to 12, wherein the one or more pharmaceutically acceptable excipients comprise about 2%, about 3%, about 4%, about 5%, about 6%, about 7%, about 8%, about 9%, about 10%, about 11%, or about 12% w/w sodium starch glycolate.

15. The formulation of any one of claims 1 to 14, wherein the one or more pharmaceutically acceptable excipients comprise about 0.01% to about 10% w/w magnesium stearate.

16. The formulation of any one of claims 1 to 14, wherein the one or more pharmaceutically acceptable excipients comprise about 0.1%, about 0.25%, about 0.5%, about 0.75%, about 1%, about 1.25%, about 1.5%, about 1.75%, about 2%, about 3%, about 4%, or about 5% w/w magnesium stearate.

17. The formulation of any one of claims 1 to 16, wherein the one or more pharmaceutically acceptable excipients comprise about 0.01% to about 10% w/w colloidal silicon dioxide.

18. The formulation of any one of claims 1 to 16, wherein the one or more pharmaceutically acceptable excipients comprise about 0.1%, about 0.25%, about 0.5%, about 0.75%, about 1%, about 1.25%, about 1.5%, about 1.75%, about 2%, about 3%, about 4%, or about 5% w/w colloidal silicon dioxide.

19. The formulation of any one of claims 1 to 18, wherein the formulation is comprised into a tablet.

20. The formulation of claim 19, wherein the tablet is coated with a film coating.

21. The formulation of any one of claims 1 to 20, wherein voruciclib malonate comprises a crystal form of voruciclib malonate characterized by an X-ray powder diffraction pattern comprising one or more peaks selected from 7.30°±0.2°, 13.58°±0.2°, 14.06°±0.2°, 15.18°±0.2°, 15.66°±0.2°, 17.50°±0.2°, 18.94°±0.2°, 19.54°±0.2°, 22.22°±0.2°, 23.38° #0.2°, 24.10°±0.2°, 24.98°±0.2°, 25.94°±0.2°, 27.26°±0.2°, 28.50°±0.2°, and 32.82°±0.2° 2θ.

22. The formulation any one of claims 1 to 20, wherein voruciclib malonate comprises a crystal form of voruciclib malonate characterized by an X-ray powder diffraction pattern comprising one or more peaks selected from 6.36°±0.2° 2θ, 13.88°±0.2° 2θ, 7.31°±0.2° 20, 9.34°±0.2° 2θ, 10.05°±0.2° 2θ, 13.59°±0.2° 2θ, 14.08°±0.2° 2θ, 15.21°±0.2° 2θ, 15.67°±0.2° 2θ, 17.53°±0.2° 2θ, 18.70°±0.2° 2θ, 18.98°±0.2° 2θ, 19.38°±0.2° 2θ, 19.67°±0.2° 2θ, 20.16°±0.2° 2θ, 20.39°±0.2° 2θ, 21.01°±0.2° 2θ, 22.27°±0.2° 2θ, 23.35°±0.2° 2θ, 24.15°±0.2° 2θ, 24.67°±0.2° 2θ, 25.00°±0.2° 2θ, 25.18°±0.2° 2θ, 25.57°±0.2° 2θ, 25.93°±0.2° 2θ, 26.21°±0.2° 2θ, 27.19°±0.2° 2θ, and 27.38°±0.2° 2θ.

23. The formulation of claim 21 or 22, wherein the crystal form is a crystalline anhydrate.

24. The formulation of claim 21 or 22, wherein the crystal form is a crystalline hydrate.

25. A method of treating a disease or disorder in a subject, the method comprising administering to the subject a therapeutically effective of a formulation of any one of claims 1 to 24.

26. A method of treating a disease or disorder in a subject, the method comprising administering to the subject a therapeutically effective of a formulation comprising between about 15% to 35% w/w voruciclib malonate, about 5% to 37% w/w microcrystalline cellulose, about 1% to about 48% w/w lactose monohydrate, about 20% to about 70% w/w dibasic calcium phosphate dihydrate, about 0.1% to about 15% w/w sodium bicarbonate, about 1% to about 20% w/w sodium starch glycolate, and about 0.01% to about 10% w/w magnesium stearate.

27. The method of claim 26, wherein the voruciclib malonate comprises a crystal form of voruciclib malonate characterized by an X-ray powder diffraction pattern comprising one or more peaks selected from 7.30°±0.2°, 13.58°±0.2°, 14.06°±0.2°, 15.18°±0.2°, 15.66°±0.2°, 17.50°±0.2°, 18.94°±0.2°, 19.54°±0.2°, 22.22°±0.2°, 23.38°±0.2°, 24.10°±0.2°, 24.98°±0.2°, 25.94°±0.2°, 27.26°±0.2°, 28.50°±0.2°, and 32.82°±0.2° 2θ.

28. The method of claim 26 or 27, wherein the voruciclib malonate comprises a crystal form of voruciclib malonate characterized by an X-ray powder diffraction pattern comprising one or more peaks selected from 6.36°±0.2° 2θ, 13.88°±0.2° 2θ, 7.31°±0.2° 2θ, 9.34°±0.2° 2θ, 10.05°±0.2° 2θ, 13.59°±0.2° 2θ, 14.08°±0.2° 2θ, 15.21°±0.2° 2θ, 15.67°±0.2° 20, 17.53°±0.2° 2θ, 18.70°±0.2° 2θ, 18.98°±0.2° 2θ, 19.38°±0.2° 2θ, 19.67°±0.2° 2θ, 20.16°±0.2° 2θ, 20.39°±0.2° 2θ, 21.01°±0.2° 2θ, 22.27°±0.2° 2θ, 23.35°±0.2° 2θ, 24.15°±0.2° 2θ, 24.67°±0.2° 2θ, 25.00°±0.2° 2θ, 25.18°±0.2° 2θ, 25.57°±0.2° 2θ, 25.93°±0.2° 2θ, 26.21°±0.2° 2θ, 27.19°±0.2° 2θ, and 27.38°±0.2° 2θ.

29. The method of any one of claims 26 to 28, wherein the formulation comprises about 0.01% to about 10% w/w colloidal silicon dioxide.

30. The method of any one of claims 26 to 29, wherein the formulation is comprised into a tablet and the tablet is coated with a film coating.

31. The method of any one of claims 25 to 30, wherein the disease or disorder is a blood cancer.

32. The method of claim 31, wherein the blood cancer is selected from acute myeloid leukemia (AML), chronic myeloid leukemia (CML), acute lymphocytic lymphoma (ALL), chronic lymphocytic leukemia (CLL), non-Hodgkin lymphoma, B-cell lymphoma, diffuse large B-cell lymphoma (DLBCL), primary mediastinal B-cell lymphoma, intravascular large B-cell lymphoma, follicular lymphoma, small lymphocytic lymphoma (SLL), mantle cell lymphoma, marginal zone B-cell lymphomas, extranodal marginal zone B-cell lymphomas, nodal marginal zone B-cell lymphoma, splenic marginal zone B-cell lymphoma, Burkitt lymphoma, lymphoplasmacytic lymphoma, primary central nervous system lymphoma, T-cell lymphoma, precursor T-lymphoblastic lymphoma, peripheral T-cell lymphomas, cutaneous T-cell lymphomas, adult T-cell lymphoma, smoldering chronic adult T-cell lymphoma, acute adult T-cell lymphoma, angioimmunoblastic T-cell lymphoma, extranodal natural killer/T-cell lymphoma, nasal type, enteropathy-associated intestinal T-cell lymphoma (EATL) with subtypes I and II, and anaplastic large cell lymphoma (ALCL).

33. The method of any one of claims 25 to 32, wherein the formulation is administered to the subject such that subject receives a daily voruciclib dose between about 50 mg and about 100 mg, between about 100 mg and about 150 mg, between about 150 mg and about 200 mg, between about 200 mg and about 250 mg, between about 250 mg and about 300 mg, between about 300 mg and about 350 mg, between about 350 mg and about 400 mg, between about 400 mg and about 450 mg, between about 450 mg and about 500 mg, between about 500 mg and about 550 mg, between about 550 mg and about 600 mg, between about 600 mg and about 650 mg, between about 650 mg and about 700 mg, between about 700 mg and about 750 mg, between about 750 mg and about 800 mg, between about 800 mg and about 850 mg, between about 850 mg and about 900 mg, between about 900 mg and about 950 mg, or between about 950 mg and about 1,000 mg.

34. The method of any one of claims 25 to 32, wherein the formulation is administered to the subject such that subject receives a daily voruciclib dose of about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1,000 mg.

35. The method of any one of claims 25 to 32, wherein the formulation is administered to the subject such that subject receives a daily voruciclib dose of about 200 mg or about 250 mg.

36. The method of any one of claims 25 to 32, wherein the formulation is administered to the subject such that subject receives a daily voruciclib dose not exceeding 350 mg.

37. The method of any one of claims 33 to 36, wherein the voruciclib dose is a voruciclib free base dose.

38. The method of any one of claims 25 to 37, wherein the formulation is administered to the subject daily for about one day, about two days, about three days, about 4 days, about 5 days, about 6 days, about 7 days, about 8 days, about 9 days, about 10 days, about 11 days, about 12 days, about 13 days, or about 14 days.

39. The method of any one of claims 25 to 37, wherein the formulation is administered to the subject daily for about one week, about two weeks, about three weeks, or about 4 weeks.

40. The method of any one of claims 25 to 39, wherein administration of the formulation is paused for about one day, about two days, about three days, about 4 days, about 5 days, about 6 days, about 7 days, about 8 days, about 9 days, about 10 days, about 11 days, about 12 days, about 13 days, or about 14 days.

41. The method of any one of claims 25 to 39, wherein administration of the formulation is paused for about one week, about two weeks, about three weeks, or about 4 weeks.

42. The method of any one of claims 25 to 41, wherein the formulation is administered to the subject on a 14 days on/14 days off schedule.

43. The method of any one of claims 25 to 42, wherein the formulation is administered for about one month, about two months, about three months, about 4 months, about 5 months, about 6 months, about 7 months, about 8 months, about 9 months, about 10 months, about 11 months, or about 12 months.

44. The method of any one of claims 25 to 43, wherein the formulation is administered in combination with a BCL-2 inhibitor.

45. The method of claim 44, wherein the BCL-2 inhibitor is selected from navitoclax, venetoclax, A-1155463, A-1331852, ABT-737, obatoclax, S44563, TW-37, A-1210477, AT101, HA14-1, BAM7, sabutoclax, UMI-77, gambogic acid, maritoclax, MIMI, methylprednisolone, iMAC2, Bax inhibitor peptide V5, Bax inhibitor peptide P5, Bax channel blocker, ARRY 520 trifluoroacetate, or a pharmaceutically acceptable salt of any one thereof.

46. The method of claim 44, wherein the BCL-2 inhibitor is venetoclax or a pharmaceutically acceptable salt thereof.

47. The method of any one of claims 44 to 46, wherein the BCL-2 inhibitor is administered to the subject daily for about one day, about two days, about three days, about 4 days, about 5 days, about 6 days, about 7 days, about 8 days, about 9 days, about 10 days, about 11 days, about 12 days, about 13 days, or about 14 days.

48. The method of any one of claims 44 to 46, wherein the BCL-2 inhibitor is administered to the subject daily for about one week, about two weeks, about three weeks, or about 4 weeks.

49. The method of any one of claims 44 to 48, wherein administration of the BCL-2 inhibitor is paused for about one day, about two days, about three days, about 4 days, about 5 days, about 6 days, about 7 days, about 8 days, about 9 days, about 10 days, about 11 days, about 12 days, about 13 days, or about 14 days.

50. The method of any one of claims 44 to 48, wherein administration of the BCL-2 inhibitor is paused for about one week, about two weeks, about three weeks, or about 4 weeks.

51. The method of any one of claims 44 to 50, wherein the BCL-2 inhibitor is administered to the subject on a 14 days on/14 days off schedule.

52. The method of any one of claims 44 to 51, wherein the BCL-2 inhibitor is administered to the subject for about one month, about two months, about three months, about 4 months, about 5 months, about 6 months, about 7 months, about 8 months, about 9 months, about 10 months, about 11 months, or about 12 months.
